SEQUENCE LISTING (46 sequences)

SEQ ID NO: 1 - protein, 1240 aa, synthetic construct
MASAAPLVTAMCLLGNVSFPCDRPPTCYTREPSRALDILEENVNHEAYDTLLNAILRCGSSGRSKRSVIDDFTLTSPYLGTCSYCHHTVPCFSPVKIEQVWDEADDNTIRIQTSAQFGYDQSGAASANKYRYMAAAAVTAAGHVGQVQLVQSGAEDKKPGASVKVSCKASGFNIKDTYIHWVRQAPGQGLEWMGRIDPANDNTLYASKFQGRVTITADTSSNTAYMELSSLRSEDTAVYYCGRGYGYYVFDHWGQGTTVTVSSSSGGGGSGGGGGGSSRSSDIVMTQSPSSLSASVGDRVTITCRTSRSISQYLAWYQEKPGKAPKLLIYSGSTLQSGVPSRFSGSGSGTDFTLTISSLQPEDFATYYCQQHNENPLTFGQGTKVEIKGVHGAAVTTVKEGTMDDIKISTSGPCRRLSYKGYFLLAKCPPGDSVTVSIVSSNSATSCTLARKIKPKFVGREKYDLPPVHGKKIPCTVYDRLAATTAGYITMHRPRPHAYTSYLEESSGKVYAKPPSGKNITYECKCGDYKTGTVSTRTEITGCTAIKQCVAYKSDQTKWVFNSPDLIRHDDHTAQGKLHLPFKLIPSTCMVPVAHAPNVIHGFKHISLQLDTDHLTLLTTRRLGANPEPTTEWIVGKTVRNFTVDRDGLEYIWGNHEPVRVYAQESAPGDPHGWPHEIVQHYYHRHPVYTILAVASATVAMMIGVTVAVLCACKARRECLTPYALAPNAVIPTSLALLCCVRSANAETFTETMSYLWSNSQPFFWVQLCIPLAAFIVLMRCCSCCLPFLVVAGAYLAKVDAYEHATTVPNVPQIPYKALVERAGYAPLNLEITVMSSEVLPSTNQEYITCKFTTVVPSPKIKCCGSLECQPAAHADYTCKVFGGVYPFMWGGAQCFCDSENSQMSEAYVELSADCASDHAQAIKVHTAAMKVGLRIVYGNTTSFLDVYVNGVTPGTSKDLKVIAGPISASFTPFDHKVVIHRGLVYNYDFPEYGAMKPGAFGDIQATSLTSKDLIASTDIRLLKPSYGNVHVPYTQASSGFEMWKNNSGRPLQETAPFGCKIAVNPLRAVDCSYGNIPISIDIPNAAFIRTSDAPLVSTVKCEVSECTYSADFGGMATLQYVSDREGQCPVHSHSSTATLQESTVHVLEKGAVTVHFSTASPQANFIVSLCGKKTTCNAECKPPADHIVSTPHKNDQEFQAAISKTSWSWLFALFGGASSLLIIGLMIFACSMMLTSTRR

SEQ ID NO: 2 - protein, 255 aa, synthetic construct
AAGHVGQVQLVQSGAEDKKPGASVKVSCKASGFNIKDTYIHWVRQAPGQGLEWMGRIDPANDNTLYASKFQGRVTITADTSSNTAYMELSSLRSEDTAVYYCGRGYGYYVFDHWGQGTTVTVSSSSGGGGSGGGGGGSSRSSDIVMTQSPSSLSASVGDRVTITCRTSRSISQYLAWYQEKPGKAPKLLIYSGSTLQSGVPSRFSGSGSGTDFTLTISSLQPEDFATYYCQQHNENPLTFGQGTKVEIKGVHGAA

SEQ ID NO: 3 - protein, 264 aa, synthetic construct
GGGGSGGGGSQVQLQQSGAELARPGASVKMSCKASGYTFTRYTMHWVKQRPGQGLEWIGYINPSRGYTNYNQKFKDKATLTTDKSSSTAYMQLSSLTSEDSAVYYCARYYDDHYCLDYWGQGTTLTVSSSSGGGGSGGGGGGSSRSSQIVLTQSPAIMSASPGEKVTMTCSASSSVSYMNWYQQKSGTSPKRWIYDTSKLASGVPAHFRGSGSGTSYSLTISGMEAEDAATYYCQQWSSNPFTFGSGTKLEINRSGGGGSGGGG

SEQ ID NO: 4 - protein, 256 aa, synthetic construct
AAGHVGQVQLQQSGAELARPGASVKMSCKASGYTFTRYTMHWVKQRPGQGLEWIGYINPSRGYTNYNQKFKDKATLTTDKSSSTAYMQLSSLTSEDSAVYYCARYYDDHYCLDYWGQGTTLTVSSSSGGGGSGGGGGGSSRSSQIVLTQSPAIMSASPGEKVTMTCSASSSVSYMNWYQQKSGTSPKRWIYDTSKLASGVPAHFRGSGSGTSYSLTISGMEAEDAATYYCQQWSSNPFTFGSGTKLEINRGVHGAA

SEQ ID NO: 5 - protein, 263 aa, synthetic construct
GGGGSGGGGSQVQLQQSGAELARPGASVKMSCKASGYTFTRYTMHWVKQRPGQGLEWIGYINPSRGYTNYNQKFKDKATLTTDKSSSTAYMQLSSLTSEDSAVYYCARYYDDHYCLDYWGQGTTLTVSSSSGGGGSGGGGGGSSRSSSIVLTQSPAIMSASPGEKVTMTCSASSSVSYMNWYQQKSGTSPKRWIYDTSKLASGVPAHFRGSGSGTSYSLTISGMEAEDAATYYCQQWSSNPFTFGSGTKLEINSGGGGSGGGG

SEQ ID NO: 6 - protein, 256 aa, synthetic construct; identical to SEQ ID NO: 4

SEQ ID NO: 7 - protein, 10 aa, synthetic construct
GGGGSGGGGS

SEQ ID NO: 8 - protein, 10 aa, synthetic construct
SGGGGSGGGG

SEQ ID NO: 9 - protein, 6 aa, synthetic construct
AAGHVG

SEQ ID NO: 10 - protein, 6 aa, synthetic construct
GVHGAA

SEQ ID NO: 11 - protein, 18 aa, synthetic construct
SSGGGGSGGGGGGSSRSS

SEQ ID NO: 12 - protein, 1738 aa, Murine leukemia virus
MGQTVTTPLSLTLGHWKDVERIAHNQSVDVKKRRWVTFCSAEWPTFNVGWPRDGTFNRDLITQVKIKVFSPGPHGHPDQVPYIVTWEALAFDPPPWVKPFVHPKPPPPLPPSAPSLPLEPPRSTPPRSSLYPALTPSLGAKPKPQVLSDSGGPLIDLLTEDPPPYRDPRPPPSDRDGNGGEATPAGEAPDPSPMASRLRGRREPPVADSTTSQAFPLRAGGNGQLQYWPFSSSDLYNWKNNNPSFSEDPGKLTALIESVLITHQPTWDDCQQLLGTLLTGEEKQRVLLEARKAVRGDDGRPTQLPNEVDAAFPLERPDWDYTTQAGRNHLVHYRQLLLAGLQNAGRSPTNLAKVKGITQGPNESPSAFLERLKEAYRRYTPYDPEDPGQETNVSMSFIWQSAPDIGRKLERLEDLKNKTLGDLVREAEKIFNKRETPEEREERIRRETEEKEERRRTEDEQKEKERDRRRHREMSKLLATVVSGQKQDRQGGERRRSQLDRDQCAYCKEKGHWAKDCPKKPRGPRGPRPQTSLLTLDDQGGQGQEPPPEPRITLKVGGQPVTFLVDTGAQHSVLTQNPGPLSDKSAWVQGATGGKRYRWTTDRKVHLATGKVTHSFLHVPDCPYPLLGRDLLTKLKAQIHFEGSGAQVMGPMGQPLQVLTLNIEDEYRLHETSKEPDVSLGSTWLSDFPQAWAETGGMGLAVRQAPLIIPLKATSTPVSIKQYPMSQEARLGIKPHIQRLLDQGILVPCQSPWNTPLLPVKKPGTNDYRPVQDLREVNKRVEDIHPTVPNPYNLLSGLPPSHQWYTVLDLKDAFFCLRLHPTSQPLFAFEWRDPEMGISGQLTWTRLPQGFKNSPTLFDEALHRDLADFRIQHPDLILLQYVDDLLLAATSELDCQQGTRALLQTLGNLGYRASAKKAQICQKQVKYLGYLLKEGQRWLTEARKETVMGQPTPKTPRQLREFLGTAGFCRLWIPGFAEMAAPLYPLTKTGTLFNWGPDQQKAYQEIKQALLTAPALGLPDLTKPFELFVDEKQGYAKGVLTQKLGPWRRPVAYLSKKLDPVAAGWPPCLRMVAAIAVLTKDAGKLTMGQPLVILAPHAVEALVKQPPDRWLSNARMTHYQALLLDTDRVQFGPVVALNPATLLPLPEEGLQHNCLDILAEAHGTRPDLTDQPLPDADHTWYTDGSSLLQEGQRKAGAAVTTETEVIWAKALPAGTSAQRAELIALTQALKMAEGKKLNVYTDSRYAFATAHIHGEIYRRRGLLTSEGKEIKNKDEILALLKALFLPKRLSIIHCPGHQKGHSAEARGNRMADQAARKAAITETPDTSTLLIENSSPYTSEHFHYTVTDIKDLTKLGAIYDKTKKYWVYQGKPVMPDQFTFELLDFLHQLTHLSFSKMKALLERSHSPYYMLNRDRTLKNITETCKACAQVNASKSAVKQGTRVRGHRPGTHWEIDFTEIKPGLYGYKYLLVFIDTFSGWIEAFPTKKETAKVVTKKLLEEIFPRFGMPQVLGTDNGPAFVSKVSQTVADLLGIDWKLHCAYRPQSSGQVERMNRTIKETLTKLTLATGSRDWVLLLPLALYRARNTPGPHGLTPYEILYGAPPPLVNFPDPDMTRVTNSPSLQAHLQALYLVQHEVWRPLAAAYQEQLDRPVVPHPYRVGDTVWVRRHQTKNLEPRWKGPYTVLLTTPTALKVDGIAAWIHAAHVKAADPGGGPSSRLTWRVQRSQNPLKIRLTREAP

SEQ ID NO: 13 - DNA, 5217 bp, Murine leukemia virus
  source 1..5217: mol_type = genomic DNA
atgggccagactgttaccactcccttaagtttgaccttaggtcactggaaagatgtcgagcggatcgctcacaaccagtcggtagatgtcaagaagagacgttgggttaccttctgctctgcagaatggccaacctttaacgtcggatggccgcgagacggcacctttaaccgagacctcatcacccaggttaagatcaaggtcttttcacctggcccgcatggacacccagaccaggtcccctacatcgtgacctgggaagccttggcttttgacccccctccctgggtcaagccctttgtacaccctaagcctccgcctcctcttcctccatccgccccgtctctcccccttgaacctcctcgttcgaccccgcctcgatcctccctttatccagccctcactccttctctaggcgccaaacctaaacctcaagttctttctgacagtggggggccgctcatcgacctacttacagaagaccccccgccttatagggacccaagaccacccccttccgacagggacggaaatggtggagaagcgacccctgcgggagaggcaccggacccctccccaatggcatctcgcctacgtgggagacgggagccccctgtggccgactccactacctcgcaggcattccccctccgcgcaggaggaaacggacagcttcaatactggccgttctcctcttctgacctttacaactggaaaaataataacccttctttttctgaagatccaggtaaactgacagctctgatcgagtctgtcctcatcacccatcagcccacctgggacgactgtcagcagctgttggggactctgctgaccggagaagaaaaacaacgggtgctcttagaggctagaaaggcggtgcggggcgatgatgggcgccccactcaactgcccaatgaagtcgatgccgcttttcccctcgagcgcccagactgggattacaccacccaggcaggtaggaaccacctagtccactatcgccagttgctcctagcgggtctccaaaacgcgggcagaagccccaccaatttggccaaggtaaaaggaataacacaagggcccaatgagtctccctcggccttcctagagagacttaaggaagcctatcgcaggtacactccttatgaccctgaggacccagggcaagaaactaatgtgtctatgtctttcatttggcagtctgccccagacattgggagaaagttagagaggttagaagatttaaaaaacaagacgcttggagatttggttagagaggcagaaaagatctttaataaacgagaaaccccggaagaaagagaggaacgtatcaggagagaaacagaggaaaaagaagaacgccgtaggacagaggatgagcagaaagagaaagaaagagatcgtaggagacatagagagatgagcaagctattggccactgtcgttagtggacagaaacaggatagacagggaggagaacgaaggaggtcccaactcgatcgcgaccagtgtgcctactgcaaagaaaaggggcactgggctaaagattgtcccaagaaaccacgaggacctcggggaccaagaccccagacctccctcctgaccctagatgactagggaggtcagggtcaggagcccccccctgaacccaggataaccctcaaagtcggggggcaacccgtcaccttcctggtagatactggggcccaacactccgtgctgacccaaaatcctggacccctaagtgataagtctgcctgggtccaaggggctactggaggaaagcggtatcgctggaccacggatcgcaaagtacatctagctaccggtaaggtcacccactctttcctccatgtaccagactgtccctatcctctgttaggaagagatttgctgactaaactaaaagcccaaatccactttgagggatcaggagctcaggttatgggaccaatggggcagcccctgcaagtgttgaccctaaatatagaagatgagtatcggctacatgagacctcaaaagagccagatgtttctctagggtccacatggctgtctgattttcctcaggcctgggcggaaaccgggggcatgggactggcagttcgccaagctcctctgatcatacctctgaaagcaacctctacccccgtgtccataaaacaataccccatgtcacaagaagccagactggggatcaagccccacatacagagactgttggaccagggaatactggtaccctgccagtccccctggaacacgcccctgctacccgttaagaaaccagggactaatgattataggcctgtccaggatctgagagaagtcaacaagcgggtggaagacatccaccccaccgtgcccaacccttacaacctcttgagcgggctcccaccgtcccaccagtggtacactgtgcttgatttaaaggatgcctttttctgcctgagactccaccccaccagtcagcctctcttcgcctttgagtggagagatccagagatgggaatctcaggacaattgacctggaccagactcccacagggtttcaaaaacagtcccaccctgtttgatgaggcactgcacagagacctagcagacttccggatccagcacccagacttgatcctgctacagtacgtggatgacttactgctggccgccacttctgagctagactgccaacaaggtactcgggccctgttacaaaccctagggaacctcgggtatcgggcctcggccaagaaagcccaaatttgccagaaacaggtcaagtatctggggtatcttctaaaagagggtcagagatggctgactgaggccagaaaagagactgtgatggggcagcctactccgaagacccctcgacaactaagggagttcctagggacggcaggcttctgtcgcctctggatccctgggtttgcagaaatggcagcccccttgtaccctctcaccaaaacggggactctgtttaattggggcccagaccaacaaaaggcctatcaagaaatcaagcaagctcttctaactgccccagccctggggttgccagatttgactaagccctttgaactctttgtcgacgagaagcagggctacgccaaaggtgtcctaacgcaaaaactgggaccttggcgtcggccggtggcctacctgtccaaaaagctagacccagtagcagctgggtggcccccttgcctacggatggtagcagccattgccgtactgacaaaggatgcaggcaagctaaccatgggacagccactagtcattctggccccccatgcagtagaggcactagtcaaacaaccccccgaccgctggctttccaacgcccggatgactcactatcaggccttgcttttggacacggaccgggtccagttcggaccggtggtagccctgaacccggctacgctgctcccactgcctgaggaagggctgcaacacaactgccttgatatcctggccgaagcccacggaacccgacccgacctaacggaccagccgctcccagacgccgaccacacctggtacacggatggaagcagtctcttacaagagggacagcgtaaggcgggagctgcggtgaccaccgagaccgaggtaatctgggctaaagccctgccagccgggacatccgctcagcgggctgaactgatagcactcacccaggccctaaagatggcagaaggtaagaagctaaatgtttatactgatagccgttatgcttttgctactgcccatatccatggagaaatatacagaaggcgtgggttgctcacatcagaaggcaaagagatcaaaaataaagacgagatcttggccctactaaaagccctctttctgcccaaaagacttagcataatccattgtccaggacatcaaaagggacacagcgccgaggctagaggcaaccggatggctgaccaagcggcccgaaaggcagccatcacagagactccagacacctctaccctcctcatagaaaattcatcaccctacacctcagaacattttcattacacagtgactgatataaaggacctaaccaagttgggggccatttatgataaaacaaagaagtattgggtctaccaaggaaaacctgtgatgcctgaccagtttacttttgaattattagactttcttcatcagctgactcacctcagcttctcaaaaatgaaggctctcctagagagaagccacagtccctactacatgctgaaccgggatcgaacactcaaaaatatcactgagacctgcaaagcttgtgcacaagtcaacgccagcaagtctgccgttaaacagggaactagggtccgcgggcatcggcccggcactcattgggagatcgatttcaccgagataaagcccggattgtatggctataaatatcttctagtttttatagataccttttctggctggatagaagccttcccaaccaagaaagaaaccgccaaggtcgtaaccaagaagctactagaggagatcttccccaggttcggcatgcctcaggtattgggaactgacaatgggcctgccttcgtctccaaggtgagtcagacagtggccgatctgttggggattgattggaaattacattgtgcatacagaccccaaagctcaggccaggtagaaagaatgaatagaaccatcaaggagactttaactaaattaacgcttgcaactggctctagagactgggtgctcctactccccttagccctgtaccgagcccgcaacacgccgggcccccatggcctcaccccatatgagatcttatatggggcacccccgccccttgtaaacttccctgaccctgacatgacaagagttactaacagcccctctctccaagctcacttacaggctctctacttagtccagcacgaagtctggagacctctggcggcagcctaccaagaacaactggaccgaccggtggtacctcacccttaccgagtcggcgacacagtgtgggtccgccgacaccagactaagaacctagaacctcgctggaaaggaccttacacagtcctgctgaccacccccaccgccctcaaagtagacggcatcgcagcttggatacacgccgcccacgtgaaggctgccgaccccgggggtggaccatcctctagactgacatggcgcgttcaacgctctcaaaaccccttaaaaataaggttaacccgcgaggccccctaa

SEQ ID NO: 14 - protein, 408 aa, Murine leukemia virus
IENSSPYTSEHFHYTVTDIKDLTKLGAIYDKTKKYWVYQGKPVMPDQFTFELLDFLHQLTHLSFSKMKALLERSHSPYYMLNRDRTLKNITETCKACAQVNASKSAVKQGTRVRGHRPGTHWEIDFTEIKPGLYGYKYLLVFIDTFSGWIEAFPTKKETAKVVTKKLLEEIFPRFGMPQVLGTDNGPAFVSKVSQTVADLLGIDWKLHCAYRPQSSGQVERMNRTIKETLTKLTLATGSRDWVLLLPLALYRARNTPGPHGLTPYEILYGAPPPLVNFPDPDMTRVTNSPSLQAHLQALYLVQHEVWRPLAAAYQEQLDRPVVPHPYRVGDTVWVRRHQTKNLEPRWKGPYTVLLTTPTALKVDGIAAWIHAAHVKAADPGGGPSSRLTWRVQRSQNPLKIRLTREAP

SEQ ID NO: 15 - DNA, 1224 bp, Murine leukemia virus
  source 1..1224: mol_type = genomic DNA
atagaaaattcatcaccctacacctcagaacattttcattacacagtgactgatataaaggacctaaccaagttgggggccatttatgataaaacaaagaagtattgggtctaccaaggaaaacctgtgatgcctgaccagtttacttttgaattattagactttcttcatcagctgactcacctcagcttctcaaaaatgaaggctctcctagagagaagccacagtccctactacatgctgaaccgggatcgaacactcaaaaatatcactgagacctgcaaagcttgtgcacaagtcaacgccagcaagtctgccgttaaacagggaactagggtccgcgggcatcggcccggcactcattgggagatcgatttcaccgagataaagcccggattgtatggctataaatatcttctagtttttatagataccttttctggctggatagaagccttcccaaccaagaaagaaaccgccaaggtcgtaaccaagaagctactagaggagatcttccccaggttcggcatgcctcaggtattgggaactgacaatgggcctgccttcgtctccaaggtgagtcagacagtggccgatctgttggggattgattggaaattacattgtgcatacagaccccaaagctcaggccaggtagaaagaatgaatagaaccatcaaggagactttaactaaattaacgcttgcaactggctctagagactgggtgctcctactccccttagccctgtaccgagcccgcaacacgccgggcccccatggcctcaccccatatgagatcttatatggggcacccccgccccttgtaaacttccctgaccctgacatgacaagagttactaacagcccctctctccaagctcacttacaggctctctacttagtccagcacgaagtctggagacctctggcggcagcctaccaagaacaactggaccgaccggtggtacctcacccttaccgagtcggcgacacagtgtgggtccgccgacaccagactaagaacctagaacctcgctggaaaggaccttacacagtcctgctgaccacccccaccgccctcaaagtagacggcatcgcagcttggatacacgccgcccacgtgaaggctgccgaccccgggggtggaccatcctctagactgacatggcgcgttcaacgctctcaaaaccccttaaaaataaggttaacccgcgaggccccc

SEQ ID NO: 16 - protein, 320 aa, Herpes simplex virus type 1
MASYPGHQHASAFDQAARSRGHSNRRTALRPRRQQEATEVRPEQKMPTLLRVYIDGPHGMGKTTTTQLLVALGSRDDIVYVPEPMTYWRVLGASETIANIYTTQHRLDQGEISAGDAAVVMTSAQITMGMPYAVTDAVLAPHIGGEAGSSHAPPPALTLIFDRHPIAALLCYPAARYLMGSMTPQAVLAFVALIPPTLPGTNIVLGALPEDRHIDRLAKRQRPGERLDLAMLAAIRRVYGLLANTVRYLQCGGSWREDWGQLSGTAVPPQGAEPQSNAGPRPHIGDTLFTLFRAPELLAPNGDLYNVFAWALDVLAKRLR

SEQ ID NO: 17 - protein, 12 aa, synthetic construct
LQKKLEELELDG

SEQ ID NO: 18 - protein, 18 aa, synthetic construct
GSTSGSGKPGSGEGSTKG

SEQ ID NO: 19 - protein, 15 aa, synthetic construct
GGGGSGGGGSGGGGS

SEQ ID NO: 20 - protein, 489 aa, synthetic construct
MALPVTALLLPLALLLHAARPDIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITGSTSGSGKPGSGEGSTKGEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSSTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCGLRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 21 - protein, 486 aa, synthetic construct
MALPVTALLLPLALLLHAARPDIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITGGGGSGGGGSGGGGSEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSSTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCGLRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 22 - protein, 489 aa, synthetic construct
MALPVTALLLPLALLLHAARPDIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITSSGGGGSGGGGGGSSRSSEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSSTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCGLRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 23 - protein, 19 aa, synthetic construct
ATNFSLLKQAGDVEENPGP

SEQ ID NO: 24 - protein, 12 aa, synthetic construct
LQKKLEELELDG

SEQ ID NO: 25 - protein, 388 aa, synthetic construct
MALQKKLEELELDGSYPGHQHASAFDQAARSRGHSNGSTALRPGSQQEATEVRPEQKMPTLLRVYIDGPHGMGKTTTTQLLVALGSRDDIVYVPEPMTYWRVLGASETIANIYTTQHRLDQGEISAGDAAVVMTSAQITMGMPYAVTDAVLAPHIGGEAGSSHAPPPALTLIFDRHPIAHLLCYPAARYLMGSMTPQAVLAFVALIPPTLPGTNIVLGALPEDRHIDRLAKRQRPGERLDLAMLAAIRRVYGLLANTVRYLQCGGSWREDWGQLSGTAVPPQGAEPQSNAGPRPHIGDTLFTLFRAPELLAPNGDLYNVFAWALDVLAKRLRSMHVFILDYDQSPAGCRDALLQLTSGMVQTHVTTPGSIPTICDLARTFAREMGEAN

SEQ ID NO: 26 - protein, 335 aa, Mus musculus
MCPQKLTISWFAIVLLVSPLMAMWELEKDVYVVEVDWTPDAPGETVNLTCDTPEEDDITWTSDQRHGVIGSGKTLTITVKEFLDAGQYTCHKGGETLSHSHLLLHKKENGIWSTEILKNFKNKTFLKCEAPNYSGRFTCSWLVQRNMDLKFNIKSSSSSPDSRAVTCGMASLSAEKVTLDQRDYEKYSVSCQEDVTCPTAEETLPIELALEARQQNKYENYSTSFFIRDIIKPDPPKNLQMKPLKNSQVEVSWEYPDSWSTPHSYFSLKFFVRIQRKKEKMKETEEGCNQKGAFLVEKTSTEVQCKGGNVCVQAQDRYYNSSCSKWACVPCRVRS

SEQ ID NO: 27 - protein, 18 aa, synthetic construct
EGRGSLLTCGDVEENPGP

SEQ ID NO: 28 - DNA, 298 bp, Simian virus 40
  source 1..298: mol_type = genomic DNA
ggtgtggaaagtccccaggctccccagcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccagttccgcccattctccgccccatggctgactaattttttttatttatgcagaggccgaggccgcctcggcctctgagctattccagaagtagtgaggaggcttttttgg

SEQ ID NO: 29 - protein, 215 aa, Mus musculus
MCQSRYLLFLATLALINHLSLARVIPVSGPARCLSQSRNLLKTTDDMVKTAREKLKHYSCTAEDIDHEDITRDQTSTLKTCLPLELHKNESCLATRETSSTTRGSCLPPQKTSLMMTLCLGSIYEDLKMYQTEFQAINAALQNHNHQQIILDKGMLVAIDELMQSLNHNGETLRQKPPVGEADPYRVKMKLCILLHAFSTRVVTINRVMGYLSSA

SEQ ID NO: 30 - protein, 4 aa, unidentified
  source 1..4: note = Furin cleavage site
RRKR

SEQ ID NO: 31 - protein, 328 aa, Homo sapiens
MCHQQLVISWFSLVFLASPLVAIWELKKDVYVVELDWYPDAPGEMVVLTCDTPEEDGITWTLDQSSEVLGSGKTLTIQVKEFGDAGQYTCHKGGEVLSHSLLLLHKKEDGIWSTDILKDQKEPKNKTFLRCEAKNYSGRFTCWWLTTISTDLTFSVKSSRGSSDPQGVTCGAATLSAERVRGDNKEYEYSVECQEDSACPAAEESLPIEVMVDAVHKLKYENYTSSFFIRDIIKPDPPKNLQLKPLKNSRQVEVSWEYPDTWSTPHSYFSLTFCVQVQGKSKREKKDRVFTDKTSATVICRKNASISVRAQDRYYSSSWSEWASVPCS

SEQ ID NO: 32 - protein, 219 aa, Homo sapiens
MCPARSLLLVATLVLLDHLSLARNLPVATPDPGMFPCLHHSQNLLRAVSNMLQKARQTLEFYPCTSEEIDHEDITKDKTSTVEACLPLELTKNESCLNSRETSFITNGSCLASRKTSFMMALCLSSIYEDLKMYQVEFKTMNAKLLMDPKRQIFLDQNMLAVIDELMQALNFNSETVPQKSSLEEPDFYKTKIKLCILLHAFRIRAVTIDRVMSYLNAS

SEQ ID NO: 33 - protein, 562 aa, synthetic construct
MCHQQLVISWFSLVFLASPLVAIWELKKDVYVVELDWYPDAPGEMVVLTCDTPEEDGITWTLDQSSEVLGSGKTLTIQVKEFGDAGQYTCHKGGEVLSHSLLLLHKKEDGIWSTDILKDQKEPKNKTFLRCEAKNYSGRFTCWWLTTISTDLTFSVKSSRGSSDPQGVTCGAATLSAERVRGDNKEYEYSVECQEDSACPAAEESLPIEVMVDAVHKLKYENYTSSFFIRDIIKPDPPKNLQLKPLKNSRQVEVSWEYPDTWSTPHSYFSLTFCVQVQGKSKREKKDRVFTDKTSATVICRKNASISVRAQDRYYSSSWSEWASVPCSGGGGSGGGGSGGGGSMCPARSLLLVATLVLLDHLSLARNLPVATPDPGMFPCLHHSQNLLRAVSNMLQKARQTLEFYPCTSEEIDHEDITKDKTSTVEACLPLELTKNESCLNSRETSFITNGSCLASRKTSFMMALCLSSIYEDLKMYQVEFKTMNAKLLMDPKRQIFLDQNMLAVIDELMQALNFNSETVPQKSSLEEPDFYKTKIKLCILLHAFRIRAVTIDRVMSYLNAS

SEQ ID NO: 34 - protein, 654 aa, Moloney murine leukemia virus
MARSTLSKPPQDKINPWKPLIVMGVLLGVGMAESPHQVFNVTWRVTNLMTGRTANATSLLGTVQDAFPKLYFDLCDLVGEEWDPSDQEPYVGYGCKYPAGRQRTRTFDFYVCPGHTVKSGCGGPGEGYCGKWGCETTGQAYWKPTSSWDLISLKRGNTPWDTGCSKVACGPCYDLSKVSNSFQGATRGGRCNPLVLEFTDAGKKANWDGPKSWGLRLYRTGTDPITMFSLTRQVLNVGPRVPIGPNPVLPDQRLPSSPIEIVPAPQPPSPLNTSYPPSTTSTPSTSPTSPSVPQPPPGTGDRLLALVKGAYQALNLTNPDKTQECWLCLVSGPPYYEGVAVVGTYTNHSTAPANCTATSQHKLTLSEVTGQGLCMGAVPKTHQALCNTTQSAGSGSYYLAAPAGTMWACSTGLTPCLSTTVLNLTTDYCVLVELWPRVIYHSPDYMYGQLEQRTKYKREPVSLTLALLLGGLTMGGIAAGIGTGTTALIKTQQFEQLHAAIQTDLNEVEKSITNLEKSLTSLSEVVLQNRRGLDLLFLKEGGLCAALKEECCFYADHTGLVRDSMAKLRERLNQRQKLFETGQGWFEGLFNRSPWFTTLISTIMGPLIVLLLILLFGPCILNRLVQFVKDRISVVQALVLTQQYHQLKPIEYEP

SEQ ID NO: 35 - protein, 983 aa, Sindbis virus
MASAAPLVTAMCLLGNVSFPCDRPPTCYTREPSRALDILEENVNHEAYDTLLNAILRCGSSGRSKRSVIDDFTLTSPYLGTCSYCHHTVPCFSPVKIEQVWDEADDNTIRIQTSAQFGYDQSGAASANKYRYMSLKQDHTVKEGTMDDIKISTSGPCRRLSYKGYFLLAKCPPGDSVTVSIVSSNSATSCTLARKIKPKFVGREKYDLPPVHGKKIPCTVYDRLKETTAGYITMHRPRPHAYTSYLEESSGKVYAKPPSGKNITYECKCGDYKTGTVSTRTEITGCTAIKQCVAYKSDQTKWVFNSPDLIRHDDHTAQGKLHLPFKLIPSTCMVPVAHAPNVIHGFKHISLQLDTDHLTLLTTRRLGANPEPTTEWIVGKTVRNFTVDRDGLEYIWGNHEPVRVYAQESAPGDPHGWPHEIVQHYYHRHPVYTILAVASATVAMMIGVTVAVLCACKARRECLTPYALAPNAVIPTSLALLCCVRSANAETFTETMSYLWSNSQPFFWVQLCIPLAAFIVLMRCCSCCLPFLVVAGAYLAKVDAYEHATTVPNVPQIPYKALVERAGYAPLNLEITVMSSEVLPSTNQEYITCKFTTVVPSPKIKCCGSLECQPAAHADYTCKVFGGVYPFMWGGAQCFCDSENSQMSEAYVELSADCASDHAQAIKVHTAAMKVGLRIVYGNTTSFLDVYVNGVTPGTSKDLKVIAGPISASFTPFDHKVVIHRGLVYNYDFPEYGAMKPGAFGDIQATSLTSKDLIASTDIRLLKPSAKNVHVPYTQASSGFEMWKNNSGRPLQETAPFGCKIAVNPLRAVDCSYGNIPISIDIPNAAFIRTSDAPLVSTVKCEVSECTYSADFGGMATLQYVSDREGQCPVHSHSSTATLQESTVHVLEKGAVTVHFSTASPQANFIVSLCGKKTTCNAECKPPADHIVSTPHKNDQEFQAAISKTSWSWLFALFGGASSLLIIGLMIFACSMMLTSTRR

SEQ ID NO: 36 - protein, 270 aa, synthetic construct
GGGGSGGGGSEVQLVESGGGLVQPGRSLRLSCAASGFTFDDYTMHWVRQAPGKGLEWVSGISWNSGSIGYADSVKGRFTISRDNAKKSLYLQMNSLRAEDTALYYCAKDNSGYGHYYYGMDVWGQGTTVTVASASTKGGGGGSGGGGSGGGGSEIVMTQSPATLSVSPGERATLSCRASQSVSSNLAWYQQKPGQAPRLLIYGASTRATGIPARFSGSGSGTEFTLTISSLQSEDFAVYYCQHYINWPLTFGGGTKVEIKSGGGGSGGGG

SEQ ID NO: 37 - protein, 262 aa, synthetic construct
AAGHVGEVQLVESGGGLVQPGRSLRLSCAASGFTFDDYTMHWVRQAPGKGLEWVSGISWNSGSIGYADSVKGRFTISRDNAKKSLYLQMNSLRAEDTALYYCAKDNSGYGHYYYGMDVWGQGTTVTVASASTKGGGGGSGGGGSGGGGSEIVMTQSPATLSVSPGERATLSCRASQSVSSNLAWYQQKPGQAPRLLIYGASTRATGIPARFSGSGSGTEFTLTISSLQSEDFAVYYCQHYINWPLTFGGGTKVEIKGVHGAA

SEQ ID NO: 38 - protein, 899 aa, synthetic construct
MALPVTALLLPLALLLHAARPDIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITSSGGGGSGGGGGGSSRSSEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSSTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCGLRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRGSGATNFSLLKQAGDVEENPGPMALQKKLEELELDGSYPGHQHASAFDQAARSRGHSNGSTALRPGSQQEATEVRPEQKMPTLLRVYIDGPHGMGKTTTTQLLVALGSRDDIVYVPEPMTYWRVLGASETIANIYTTQHRLDQGEISAGDAAVVMTSAQITMGMPYAVTDAVLAPHIGGEAGSSHAPPPALTLIFDRHPIAHLLCYPAARYLMGSMTPQAVLAFVALIPPTLPGTNIVLGALPEDRHIDRLAKRQRPGERLDLAMLAAIRRVYGLLANTVRYLQCGGSWREDWGQLSGTAVPPQGAEPQSNAGPRPHIGDTLFTLFRAPELLAPNGDLYNVFAWALDVLAKRLRSMHVFILDYDQSPAGCRDALLQLTSGMVQTHVTTPGSIPTICDLARTFAREMGEAN

SEQ ID NO: 39 - DNA, 2700 bp, synthetic construct
  source 1..2700: mol_type = other DNA
atggccttaccagtgaccgccttgctcctgccgctggccttgctgctccacgccgccaggccggacatccagatgacacagactacatcctccctgtctgcctctctgggagacagagtcaccatcagttgcagggcaagtcaggacattagtaaatatttaaattggtatcagcagaaaccagatggaactgttaaactcctgatctaccatacatcaagattacactcaggagtcccatcaaggttcagtggcagtgggtctggaacagattattctctcaccattagcaacctggagcaagaagatattgccacttacttttgccaacagggtaatacgcttccgtacacgttcggaggggggaccaagctggagatcacatcctctggcggcggcggctctggcggcggaggaggaggcagctccaggtctagcgaggtgaaactgcaggagtcaggacctggcctggtggcgccctcacagagcctgtccgtcacatgcactgtctcaggggtctcattacccgactatggtgtaagctggattcgccagcctccacgaaagggtctggagtggctgggagtaatatggggtagtgaaaccacatactataattcagctctcaaatccagactgaccatcatcaaggacaactccaagagccaagttttcttaaaaatgaacagtctgcaaactgatgacacagccatttactactgtgccaaacattattactacggtggtagctatgctatggactactggggccaaggaacctcagtcaccgtctcctcaaccacgacgccagcgccgcgaccaccaacaccggcgcccaccatcgcgtcgcagcccctgtccctgcgcccagaggcgtgccggccagcggcggggggcgcagtgcacacgagggggctggacttcgcctgtgatatctacatctgggcgcccttggccgggacttgtggggtccttctcctgtcactggttatcaccctttactgcaaacggggcagaaagaaactcctgtatatattcaaacaaccatttatgagaccagtacaaactactcaagaggaagatggctgtagctgccgatttccagaagaagaagaaggaggatgtggactgagagtgaagttcagcaggagcgcagacgcccccgcgtaccagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgcggctctggagcaactaacttttcactgttgaaacaggccggagatgtagaggaaaaccctggccccatggccctgcagaaaaagctggaagagctggaactggatggcagctaccccggccaccagcacgccagcgccttcgaccaggccgcccgcagccgcggccacagcaacggcagcaccgcactgcggccaggatctcagcaggaggccaccgaggtgcgccccgagcagaagatgcccaccctgctgcgcgtgtacatcgacggaccacacggcatgggcaagaccaccaccacccagctgctggtggccctgggcagccgcgacgacatcgtgtacgtgcccgagcccatgacctactggcgcgtgctgggcgccagcgagaccatcgccaacatctacaccacccagcaccgcctggaccaaggcgagatcagcgccggcgacgccgccgtggtgatgaccagcgcccagattacaatgggcatgccctacgccgtgaccgacgccgtgctggcaccacacatcggcggcgaggccggcagcagccacgcaccaccaccagcactgaccctgatcttcgaccggcacccaatcgcacacctgctgtgctacccggcagcacgctacctgatgggctccatgacaccacaagccgtgctggccttcgtggccctgatcccaccaacactgcccggcaccaacatcgtgctgggcgccctgcccgaggaccgccacatcgaccgcctggccaagcgccagcgccccggcgagcgcctggacctggccatgctggccgccatccgccgcgtgtacggcctgctggccaacaccgtgcgctacctgcagtgcggcggcagctggcgcgaggactggggccagctgagcggcaccgccgtgccaccacagggcgccgagccacagagcaacgccggaccacgaccacacatcggcgacaccctgttcaccctgttccgggcaccagagctgctggcaccaaacggcgacctgtacaacgtgttcgcctgggccctggacgtgctggccaagcgcctgcgctccatgcacgtgttcatcctggactacgaccagtcaccggccggctgccgcgacgccctgctgcagctgaccagcggcatggtgcagacccacgtgacaacacccggcagcatcccaacaatctgcgacctggcccgcaccttcgcccgcgagatgggcgaggccaactaa

SEQ ID NO: 40 - DNA, 4837 bp, synthetic construct
  source 1..4837: mol_type = other DNA
atggccttaccagtgaccgccttgctcctgccgctggccttgctgctccacgccgccaggccggacatccagatgacacagactacatcctccctgtctgcctctctgggagacagagtcaccatcagttgcagggcaagtcaggacattagtaaatatttaaattggtatcagcagaaaccagatggaactgttaaactcctgatctaccatacatcaagattacactcaggagtcccatcaaggttcagtggcagtgggtctggaacagattattctctcaccattagcaacctggagcaagaagatattgccacttacttttgccaacagggtaatacgcttccgtacacgttcggaggggggaccaagctggagatcacatcctctggcggcggcggctctggcggcggaggaggaggcagctccaggtctagcgaggtgaaactgcaggagtcaggacctggcctggtggcgccctcacagagcctgtccgtcacatgcactgtctcaggggtctcattacccgactatggtgtaagctggattcgccagcctccacgaaagggtctggagtggctgggagtaatatggggtagtgaaaccacatactataattcagctctcaaatccagactgaccatcatcaaggacaactccaagagccaagttttcttaaaaatgaacagtctgcaaactgatgacacagccatttactactgtgccaaacattattactacggtggtagctatgctatggactactggggccaaggaacctcagtcaccgtctcctcaaccacgacgccagcgccgcgaccaccaacaccggcgcccaccatcgcgtcgcagcccctgtccctgcgcccagaggcgtgccggccagcggcggggggcgcagtgcacacgagggggctggacttcgcctgtgatatctacatctgggcgcccttggccgggacttgtggggtccttctcctgtcactggttatcaccctttactgcaaacggggcagaaagaaactcctgtatatattcaaacaaccatttatgagaccagtacaaactactcaagaggaagatggctgtagctgccgatttccagaagaagaagaaggaggatgtggactgagagtgaagttcagcaggagcgcagacgcccccgcgtaccagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgcggctctggagcaactaacttttcactgttgaaacaggccggagatgtagaggaaaaccctggccccatggccctgcagaaaaagctggaagagctggaactggatggcagctaccccggccaccagcacgccagcgccttcgaccaggccgcccgcagccgcggccacagcaacggcagcaccgcactgcggccaggatctcagcaggaggccaccgaggtgcgccccgagcagaagatgcccaccctgctgcgcgtgtacatcgacggaccacacggcatgggcaagaccaccaccacccagctgctggtggccctgggcagccgcgacgacatcgtgtacgtgcccgagcccatgacctactggcgcgtgctgggcgccagcgagaccatcgccaacatctacaccacccagcaccgcctggaccaaggcgagatcagcgccggcgacgccgccgtggtgatgaccagcgcccagattacaatgggcatgccctacgccgtgaccgacgccgtgctggcaccacacatcggcggcgaggccggcagcagccacgcaccaccaccagcactgaccctgatcttcgaccggcacccaatcgcacacctgctgtgctacccggcagcacgctacctgatgggctccatgacaccacaagccgtgctggccttcgtggccctgatcccaccaacactgcccggcaccaacatcgtgctgggcgccctgcccgaggaccgccacatcgaccgcctggccaagcgccagcgccccggcgagcgcctggacctggccatgctggccgccatccgccgcgtgtacggcctgctggccaacaccgtgcgctacctgcagtgcggcggcagctggcgcgaggactggggccagctgagcggcaccgccgtgccaccacagggcgccgagccacagagcaacgccggaccacgaccacacatcggcgacaccctgttcaccctgttccgggcaccagagctgctggcaccaaacggcgacctgtacaacgtgttcgcctgggccctggacgtgctggccaagcgcctgcgctccatgcacgtgttcatcctggactacgaccagtcaccggccggctgccgcgacgccctgctgcagctgaccagcggcatggtgcagacccacgtgacaacacccggcagcatcccaacaatctgcgacctggcccgcaccttcgcccgcgagatgggcgaggccaactaaggatccctcgagatacacattgtgttctcgactcgctgtggaatgtgtgtcagttagggtgtggaaagtccccaggctccccagcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccagttccgcccattctccgccccatggctgactaattttttttatttatgcagaggccgaggccgcctcggcctctgagctattccagaagtagtgaggaggcttttttggaggcctagtcttttgcaaaaagctcgaagatcaattccgatctgatcaaccggtatgtgcccccagaagctgaccatcagctggttcgccatcgtgctgctggtgtcccctctgatggctatgtgggagctggagaaggacgtgtacgtggtggaggtggactggacaccagatgcccctggcgagaccgtgaacctgacatgcgacacccccgaggaggacgacatcacctggacatctgatcagcggcacggcgtgatcggcagcggaaagaccctgacaatcaccgtgaaggagtttctggatgctggacagtacacatgtcacaagggcggagagaccctgtctcacagccacctgctgctgcacaagaaggagaacggcatctggtccacagagatcctgaagaacttcaagaacaagacctttctgaagtgcgaggccccaaactacagcggacggttcacctgttcctggctggtgcagcgcaacatggacctgaagtttaacatcaagagcagcagcagcagccccgattctcgggctgtgacatgtggcatggcctccctgtctgctgagaaggtgaccctggaccagcgcgattacgagaagtacagcgtgtcctgccaggaggacgtgacatgtccaaccgccgaggagacactgccaatcgagctggccctggaggctaggcagcagaacaagtacgagaactactctaccagcttctttatccgcgacatcatcaagcctgatccccctaagaacctgcagatgaagccactgaagaactcccaggtcgaggtgtcttgggagtaccccgactcctggtctacacctcactcttacttcagcctgaagttcttcgtgagaatccagagaaagaaggagaagatgaaggagaccgaggagggctgcaaccagaagggagcttttctggtggagaagacaagcaccgaggtgcagtgcaagggcggaaacgtgtgcgtgcaggcccaggacaggtactacaactctagctgttccaagtgggcttgcgtgccttgtcgggtgcgctctaggagaaagaggggcagcggagagggaaggggcagcctgctgacatgcggcgatgtggaggagaaccccggacctatgtgccagagcagatacctgctgttcctggccaccctggctctgatcaaccacctgtctctggccagagtgatccctgtgagcggcccagctaggtgcctgagccagtccagaaacctgctgaagaccacagacgatatggtgaagacagccagggagaagctgaagcactactcctgtaccgctgaggacatcgatcacgaggacatcacaagagatcagacatccaccctgaagacctgcctgcctctggagctgcacaagaacgagtcttgtctggccacaagggagacctcctctaccacaagaggctcctgcctgccaccccagaagacatctctgatgatgaccctgtgcctgggaagcatctacgaggacctgaagatgtaccagacagagttccaggccatcaacgccgctctgcagaaccacaaccaccagcagatcatcctggacaagggcatgctggtggctatcgatgagctgatgcagtctctgaaccacaacggcgagaccctgaggcagaagcctccagtgggagaggccgatccatacagagtgaagatgaagctgtgcatcctgctgcacgcttttagcacacgggtggtgaccatcaaccgcgtgatgggctacctgagcagcgcctga

SEQ ID NO: 41 - DNA, 3304 bp, synthetic construct
  source 1..3304: mol_type = other DNA
atggccctgcagaaaaagctggaagagctggaactggatggcagctaccccggccaccagcacgccagcgccttcgaccaggccgcccgcagccgcggccacagcaacggcagcaccgcactgcggccaggatctcagcaggaggccaccgaggtgcgccccgagcagaagatgcccaccctgctgcgcgtgtacatcgacggaccacacggcatgggcaagaccaccaccacccagctgctggtggccctgggcagccgcgacgacatcgtgtacgtgcccgagcccatgacctactggcgcgtgctgggcgccagcgagaccatcgccaacatctacaccacccagcaccgcctggaccaaggcgagatcagcgccggcgacgccgccgtggtgatgaccagcgcccagattacaatgggcatgccctacgccgtgaccgacgccgtgctggcaccacacatcggcggcgaggccggcagcagccacgcaccaccaccagcactgaccctgatcttcgaccggcacccaatcgcacacctgctgtgctacccggcagcacgctacctgatgggctccatgacaccacaagccgtgctggccttcgtggccctgatcccaccaacactgcccggcaccaacatcgtgctgggcgccctgcccgaggaccgccacatcgaccgcctggccaagcgccagcgccccggcgagcgcctggacctggccatgctggccgccatccgccgcgtgtacggcctgctggccaacaccgtgcgctacctgcagtgcggcggcagctggcgcgaggactggggccagctgagcggcaccgccgtgccaccacagggcgccgagccacagagcaacgccggaccacgaccacacatcggcgacaccctgttcaccctgttccgggcaccagagctgctggcaccaaacggcgacctgtacaacgtgttcgcctgggccctggacgtgctggccaagcgcctgcgctccatgcacgtgttcatcctggactacgaccagtcaccggccggctgccgcgacgccctgctgcagctgaccagcggcatggtgcagacccacgtgacaacacccggcagcatcccaacaatctgcgacctggcccgcaccttcgcccgcgagatgggcgaggccaactaaggatccctcgagatacacattgtgttctcgactcgctgtggaatgtgtgtcagttagggtgtggaaagtccccaggctccccagcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccagttccgcccattctccgccccatggctgactaattttttttatttatgcagaggccgaggccgcctcggcctctgagctattccagaagtagtgaggaggcttttttggaggcctagtcttttgcaaaaagctcgaagatcaattccgatctgatcaaccggtatgtgcccccagaagctgaccatcagctggttcgccatcgtgctgctggtgtcccctctgatggctatgtgggagctggagaaggacgtgtacgtggtggaggtggactggacaccagatgcccctggcgagaccgtgaacctgacatgcgacacccccgaggaggacgacatcacctggacatctgatcagcggcacggcgtgatcggcagcggaaagaccctgacaatcaccgtgaaggagtttctggatgctggacagtacacatgtcacaagggcggagagaccctgtctcacagccacctgctgctgcacaagaaggagaacggcatctggtccacagagatcctgaagaacttcaagaacaagacctttctgaagtgcgaggccccaaactacagcggacggttcacctgttcctggctggtgcagcgcaacatggacctgaagtttaacatcaagagcagcagcagcagccccgattctcgggctgtgacatgtggcatggcctccctgtctgctgagaaggtgaccctggaccagcgcgattacgagaagtacagcgtgtcctgccaggaggacgtgacatgtccaaccgccgaggagacactgccaatcgagctggccctggaggctaggcagcagaacaagtacgagaactactctaccagcttctttatccgcgacatcatcaagcctgatccccctaagaacctgcagatgaagccactgaagaactcccaggtcgaggtgtcttgggagtaccccgactcctggtctacacctcactcttacttcagcctgaagttcttcgtgagaatccagagaaagaaggagaagatgaaggagaccgaggagggctgcaaccagaagggagcttttctggtggagaagacaagcaccgaggtgcagtgcaagggcggaaacgtgtgcgtgcaggcccaggacaggtactacaactctagctgttccaagtgggcttgcgtgccttgtcgggtgcgctctaggagaaagaggggcagcggagagggaaggggcagcctgctgacatgcggcgatgtggaggagaaccccggacctatgtgccagagcagatacctgctgttcctggccaccctggctctgatcaaccacctgtctctggccagagtgatccctgtgagcggcccagctaggtgcctgagccagtccagaaacctgctgaagaccacagacgatatggtgaagacagccagggagaagctgaagcactactcctgtaccgctgaggacatcgatcacgaggacatcacaagagatcagacatccaccctgaagacctgcctgcctctggagctgcacaagaacgagtcttgtctggccacaagggagacctcctctaccacaagaggctcctgcctgccaccccagaagacatctctgatgatgaccctgtgcctgggaagcatctacgaggacctgaagatgtaccagacagagttccaggccatcaacgccgctctgcagaaccacaaccaccagcagatcatcctggacaagggcatgctggtggctatcgatgagctgatgcagtctctgaaccacaacggcgagaccctgaggcagaagcctccagtgggagaggccgatccatacagagtgaagatgaagctgtgcatcctgctgcacgcttttagcacacgggtggtgaccatcaaccgcgtgatgggctacctgagcagcgcctga

SEQ ID NO: 42 - protein, 1493 aa, synthetic construct
MALPVTALLLPLALLLHAARPDIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITSSGGGGSGGGGGGSSRSSEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSSTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCGLRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRGSGATNFSLLKQAGDVEENPGPMALQKKLEELELDGSYPGHQHASAFDQAARSRGHSNGSTALRPGSQQEATEVRPEQKMPTLLRVYIDGPHGMGKTTTTQLLVALGSRDDIVYVPEPMTYWRVLGASETIANIYTTQHRLDQGEISAGDAAVVMTSAQITMGMPYAVTDAVLAPHIGGEAGSSHAPPPALTLIFDRHPIAHLLCYPAARYLMGSMTPQAVLAFVALIPPTLPGTNIVLGALPEDRHIDRLAKRQRPGERLDLAMLAAIRRVYGLLANTVRYLQCGGSWREDWGQLSGTAVPPQGAEPQSNAGPRPHIGDTLFTLFRAPELLAPNGDLYNVFAWALDVLAKRLRSMHVFILDYDQSPAGCRDALLQLTSGMVQTHVTTPGSIPTICDLARTFAREMGEANGSGATNFSLLKQAGDVEENPGPMCHQQLVISWFSLVFLASPLVAIWELKKDVYVVELDWYPDAPGEMVVLTCDTPEEDGITWTLDQSSEVLGSGKTLTIQVKEFGDAGQYTCHKGGEVLSHSLLLLHKKEDGIWSTDILKDQKEPKNKTFLRCEAKNYSGRFTCWWLTTISTDLTFSVKSSRGSSDPQGVTCGAATLSAERVRGDNKEYEYSVECQEDSACPAAEESLPIEVMVDAVHKLKYENYTSSFFIRDIIKPDPPKNLQLKPLKNSRQVEVSWEYPDTWSTPHSYFSLTFCVQVQGKSKREKKDRVFTDKTSATVICRKNASISVRAQDRYYSSSWSEWASVPCSRRKRGSGEGRGSLLTCGDVEENPGPMCPARSLLLVATLVLLDHLSLARNLPVATPDPGMFPCLHHSQNLLRAVSNMLQKARQTLEFYPCTSEEIDHEDITKDKTSTVEACLPLELTKNESCLNSRETSFITNGSCLASRKTSFMMALCLSSIYEDLKMYQVEFKTMNAKLLMDPKRQIFLDQNMLAVIDELMQALNFNSETVPQKSSLEEPDFYKTKIKLCILLHAFRIRAVTIDRVMSYLNAS

SEQ ID NO: 43 - protein, 1483 aa, synthetic construct
MALPVTALLLPLALLLHAARPDIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITSSGGGGSGGGGGGSSRSSEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSSTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCGLRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRGSGATNFSLLKQAGDVEENPGPMALQKKLEELELDGSYPGHQHASAFDQAARSRGHSNGSTALRPGSQQEATEVRPEQKMPTLLRVYIDGPHGMGKTTTTQLLVALGSRDDIVYVPEPMTYWRVLGASETIANIYTTQHRLDQGEISAGDAAVVMTSAQITMGMPYAVTDAVLAPHIGGEAGSSHAPPPALTLIFDRHPIAHLLCYPAARYLMGSMTPQAVLAFVALIPPTLPGTNIVLGALPEDRHIDRLAKRQRPGERLDLAMLAAIRRVYGLLANTVRYLQCGGSWREDWGQLSGTAVPPQGAEPQSNAGPRPHIGDTLFTLFRAPELLAPNGDLYNVFAWALDVLAKRLRSMHVFILDYDQSPAGCRDALLQLTSGMVQTHVTTPGSIPTICDLARTFAREMGEANGSGATNFSLLKQAGDVEENPGPMCHQQLVISWFSLVFLASPLVAIWELKKDVYVVELDWYPDAPGEMVVLTCDTPEEDGITWTLDQSSEVLGSGKTLTIQVKEFGDAGQYTCHKGGEVLSHSLLLLHKKEDGIWSTDILKDQKEPKNKTFLRCEAKNYSGRFTCWWLTTISTDLTFSVKSSRGSSDPQGVTCGAATLSAERVRGDNKEYEYSVECQEDSACPAAEESLPIEVMVDAVHKLKYENYTSSFFIRDIIKPDPPKNLQLKPLKNSRQVEVSWEYPDTWSTPHSYFSLTFCVQVQGKSKREKKDRVFTDKTSATVICRKNASISVRAQDRYYSSSWSEWASVPCSGGGGSGGGGSGGGGSMCPARSLLLVATLVLLDHLSLARNLPVATPDPGMFPCLHHSQNLLRAVSNMLQKARQTLEFYPCTSEEIDHEDITKDKTSTVEACLPLELTKNESCLNSRETSFITNGSCLASRKTSFMMALCLSSIYEDLKMYQVEFKTMNAKLLMDPKRQIFLDQNMLAVIDELMQALNFNSETVPQKSSLEEPDFYKTKIKLCILLHAFRIRAVTIDRVMSYLNAS

SEQ ID NO: 44 - DNA, 3056 bp, synthetic construct
  source 1..3056: mol_type = other DNA
atggccttaccagtgaccgccttgctcctgccgctggccttgctgctccacgccgccaggccggacatccagatgacacagactacatcctccctgtctgcctctctgggagacagagtcaccatcagttgcagggcaagtcaggacattagtaaatatttaaattggtatcagcagaaaccagatggaactgttaaactcctgatctaccatacatcaagattacactcaggagtcccatcaaggttcagtggcagtgggtctggaacagattattctctcaccattagcaacctggagcaagaagatattgccacttacttttgccaacagggtaatacgcttccgtacacgttcggaggggggaccaagctggagatcacatcctctggcggcggcggctctggcggcggaggaggaggcagctccaggtctagcgaggtgaaactgcaggagtcaggacctggcctggtggcgccctcacagcgcctgtccgtcacatgcactgtctcaggggtctcattacccgactatggtgtaagctggattcgccagcctccacgaaagggtctggagtggctgggagtaatatggggtagtgaaaccacatactataattcagctctcaaatccagactgaccatcatcaaggacaactccaagagccaagttttcttaaaaatgaacagtctgcaaactgatgacacagccatttactactgtgccaaacattattactacggtggtagctatgctatggactactggggccaaggaacctcagtcaccgtctcctcaaccacgacgccagcgccgcgaccaccaacaccggcgcccaccatcgcgtcgcagcccctgtccctgcgcccagaggcgtgccggccagcggcggggggcgcagtgcacacgagggggctggacttcgcctgtgatatctacatctgggcgcccttggccgggacttgtggggtccttctcctgtcactggttatcaccctttactgcaaacggggcagaaagaaactcctgtatatattcaaacaaccatttatgagaccagtacaaactactcaagaggaagatggctgtagctgccgatttccagaagaagaagaaggaggatgtggactgagagtgaagttcagcaggagcgcagacgcccccgcgtaccagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgctaatagggatccatcggatcccgggcccgtcgactcgctgtggaatgtgtgtcagttagggtgtggaaagtccccaggctccccagcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccagttccgcccattctccgccccatggctgactaattttttttatttatgcagaggccgaggccgcctcggcctctgagctattccagaagtagtgaggaggcttttttggaggcctaggcttttgcaaaaagctcgaagatcaattccgatctgatcaaccggtacgcgtccaccatggccctgcagaaaaagctggaagagctggaactggatggcagctaccccggccaccagcacgccagcgccttcgaccaggccgcccgcagccgcggccacagcaacggcagcaccgcactgcggccaggatctcagcaggaggccaccgaggtgcgccccgagcagaagatgcccaccctgctgcgcgtgtacatcgacggaccacacggcatgggcaagaccaccaccacccagctgctggtggccctgggcagccgcgacgacatcgtgtacgtgcccgagcccatgacctactggcgcgtgctgggcgccagcgagaccatcgccaacatctacaccacccagcaccgcctggaccaaggcgagatcagcgccggcgacgccgccgtggtgatgaccagcgcccagattacaatgggcatgccctacgccgtgaccgacgccgtgctggcaccacacatcggcggcgaggccggcagcagccacgcaccaccaccagcactgaccctgatcttcgaccggcacccaatcgcacacctgctgtgctacccggcagcacgctacctgatgggctccatgacaccacaagccgtgctggccttcgtggccctgatcccaccaacactgcccggcaccaacatcgtgctgggcgccctgcccgaggaccgccacatcgaccgcctggccaagcgccagcgccccggcgagcgcctggacctggccatgctggccgccatccgccgcgtgtacggcctgctggccaacaccgtgcgctacctgcagtgcggcggcagctggcgcgaggactggggccagctgagcggcaccgccgtgccaccacagggcgccgagccacagagcaacgccggaccacgaccacacatcggcgacaccctgttcaccctgttccgggcaccagagctgctggcaccaaacggcgacctgtacaacgtgttcgcctgggccctggacgtgctggccaagcgcctgcgctccatgcacgtgttcatcctggactacgaccagtcaccggccggctgccgcgacgccctgctgcagctgaccagcggcatggtgcagacccacgtgacaacacccggcagcatcccaacaatctgcgacctggcccgcaccttcgcccgcgagatgggcgaggccaactaa

SEQ ID NO: 45 - protein, 1247 aa, synthetic construct
MASAAPLVTAMCLLGNVSFPCDRPPTCYTREPSRALDILEENVNHEAYDTLLNAILRCGSSGRSKRSVIDDFTLTSPYLGTCSYCHHTVPCFSPVKIEQVWDEADDNTIRIQTSAQFGYDQSGAASANKYRYMAAAAVTAAGHVGEVQLVESGGGLVQPGRSLRLSCAASGFTFDDYTMHWVRQAPGKGLEWVSGISWNSGSIGYADSVKGRFTISRDNAKKSLYLQMNSLRAEDTALYYCAKDNSGYGHYYYGMDVWGQGTTVTVASASTKGGGGGSGGGGSGGGGSEIVMTQSPATLSVSPGERATLSCRASQSVSSNLAWYQQKPGQAPRLLIYGASTRATGIPARFSGSGSGTEFTLTISSLQSEDFAVYYCQHYINWPLTFGGGTKVEIKGVHGAAVTTVKEGTMDDIKISTSGPCRRLSYKGYFLLAKCPPGDSVTVSIVSSNSATSCTLARKIKPKFVGREKYDLPPVHGKKIPCTVYDRLAATTAGYITMHRPRPHAYTSYLEESSGKVYAKPPSGKNITYECKCGDYKTGTVSTRTEITGCTAIKQCVAYKSDQTKWVFNSPDLIRHDDHTAQGKLHLPFKLIPSTCMVPVAHAPNVIHGFKHISLQLDTDHLTLLTTRRLGANPEPTTEWIVGKTVRNFTVDRDGLEYIWGNHEPVRVYAQESAPGDPHGWPHEIVQHYYHRHPVYTILAVASATVAMMIGVTVAVLCACKARRECLTPYALAPNAVIPTSLALLCCVRSANAETFTETMSYLWSNSQPFFWVQLCIPLAAFIVLMRCCSCCLPFLVVAGAYLAKVDAYEHATTVPNVPQIPYKALVERAGYAPLNLEITVMSSEVLPSTNQEYITCKFTTVVPSPKIKCCGSLECQPAAHADYTCKVFGGVYPFMWGGAQCFCDSENSQMSEAYVELSADCASDHAQAIKVHTAAMKVGLRIVYGNTTSFLDVYVNGVTPGTSKDLKVIAGPISASFTPFDHKVVIHRGLVYNYDFPEYGAMKPGAFGDIQATSLTSKDLIASTDIRLLKPSYGNVHVPYTQASSGFEMWKNNSGRPLQETAPFGCKIAVNPLRAVDCSYGNIPISIDIPNAAFIRTSDAPLVSTVKCEVSECTYSADFGGMATLQYVSDREGQCPVHSHSSTATLQESTVHVLEKGAVTVHFSTASPQANFIVSLCGKKTTCNAECKPPADHIVSTPHKNDQEFQAAISKTSWSWLFALFGGASSLLIIGLMIFACSMMLTSTRR

SEQ ID NO: 46 - protein, 12 aa, synthetic construct
GGGSGGGSGGGS

(19) United States
(12) Patent Application Publication
(10) Pub. No.: US 2026/0158067 A1
(43) Pub. Date: Jun. 11, 2026

(54) VECTOR FOR EXPRESSION OF CHIMERIC ANTIGEN RECEPTOR AND USES THEREOF

(71) Applicant: GenVivo, Inc., San Marino, CA (US)

(72) Inventors: Alexander T. CUNHA, Los Angeles, CA (US); Makoto SATO, Pasadena, CA (US); Cecilia S. ROH, South Pasadena, CA (US); Robert G. JOHNSON, JR., Lafayette, CA (US)

(21) Appl. No.: 19/376,648

(22) Filed: Oct. 31, 2025

Related U.S. Application Data
(60) Provisional application No. 63/714,818, filed on Oct. 31, 2024.

(51) Int. Cl. A61K 35/17; A61K 40/11; A61K 40/31; A61K 40/42; C07K 14/005; C07K 14/54; C07K 14/705; C07K 14/725; C07K 16/28; C12N 5/0783; C12N 9/12; C12N 15/86

(52) U.S. Cl. CPC A61K 35/17; A61K 40/11; A61K 40/31; A61K 40/421; C07K 14/005; C07K 14/5434; C07K 14/7051; C07K 14/70517; C07K 14/70578; C07K 16/2809; C07K 16/2815; C12N 5/0636; C12N 9/1211; C12N 15/86; A61K 2239/13; A61K 2239/17; A61K 2239/21; C07K 2317/53; C07K 2317/622; C07K 2319/02; C07K 2319/03; C12N 2510/00; C12N 2740/13043; C12N 2770/36122; C12Y 207/01021

(57) ABSTRACT

The engineered viral vectors provided herein may comprise a modified envelope protein. In some aspects, the modified envelope protein comprises a targeting moiety. The engineered viral vectors provided herein may comprise a first payload vector encoding a chimeric antigen receptor (CAR). The engineered viral vectors may further comprise other modifications.

### CROSS-REFERENCE

This application claims the benefit of U.S. Provisional Patent Application No. 63/714,818, filed Oct. 31, 2024, which is incorporated herein by reference in its entirety.

### SEQUENCE LISTING

The instant application contains a Sequence Listing which has been submitted electronically in XML format and is hereby incorporated by reference in its entirety. Said XML copy, created on Dec. 3, 2025, is named 30863-737_601_SL.xml and is 77,324 bytes in size.

### BACKGROUND

Chimeric antigen receptor (CAR) T-cell therapy may have shown promise in treating various hematological malignancies. However, traditional methods face challenges such as limited persistence of modified T-cells and off-target effects. Ex vivo modification of patient immune cells is also costly and risky. There is a critical need for improved delivery systems that enhance T-cell modification while minimizing adverse effects.

### SUMMARY

In an aspect, provided herein are engineered viral vectors comprising a first payload vector encoding a chimeric antigen receptor (CAR).

In various embodiments of engineered viral vectors comprising a CAR herein, in some embodiments, the engineered viral vector comprises an engineered lentiviral vector or retroviral vector. In some embodiments, the engineered viral vector comprises an engineered murine leukemia viral (MLV) vector. In some embodiments, the engineered viral vector comprises an engineered murine leukemia gammaretroviral (MLV) vector. In some embodiments, the engineered viral vector comprises an engineered Moloney MLV (MoMLV) vector. In some specific embodiments, the engineered MLV vector comprises a wild-type 4070A envelope protein sequence. In some embodiments, the engineered viral vector is amphotropic. In some embodiments, the CAR comprises an intracellular signaling domain, a transmembrane domain, and an antigen recognition moiety. In some embodiments, the antigen recognition moiety binds to CD19, B-cell maturation antigen (BCMA), CD22, CD7, HER2, EGFR, Nectin-4, PMSA, or a combination thereof. In some embodiments, the antigen recognition moiety is an antigen binding portion of an antibody. In some embodiments, the antigen recognition moiety comprises at least one variable region. In some embodiments, the CAR comprises a checkpoint inhibitor. In some embodiments, the checkpoint inhibitor comprises PD-L1, CTLA-4, LAG-3, or a combination thereof.

In an aspect, provided herein are engineered viral vectors comprising a first payload vector encoding a chimeric antigen receptor (CAR) and a nucleic acid sequence encoding a deleted integrase or a defective integrase. In some embodiments, the deleted integrase does not have retroviral integration activity. In some embodiments, the defective integrase has decreased retroviral integration activity compared to a wild-type integrase. In some embodiments, the deleted integrase is a deletion of about 12%, about 15%, about 20%, about 30%, about 40%, about 50%, about 60%, about 70%, about 80%, about 90%, about 95%, about 99%, or about 100% of the integrase coding sequence. In some embodiments, the deleted integrase is a deletion of at least about 80, about 160, about 240, about 320, or about 408 residues of amino acids 1331-1738 of a murine leukemia virus Gag-Pol polyprotein.

In some embodiments, the engineered viral vector further comprises a second payload vector encoding a transgenic product. In some embodiments, the transgenic product comprises a suicide gene or a cytokine. In some embodiments, the suicide gene comprises HSV1-TK. In some embodiments, the cytokine comprises interleukin-2 (IL-2), interleukin-7 (IL-7), interleukin-12 (IL-12), interleukin-15 (IL-15), interleukin-18 (IL-18), interleukin-21 (IL-21), interferon-gamma (IFN-γ), tumor necrosis factor-alpha (TNF-α), interleukin-6 (IL-6), or a combination thereof.

In another aspect, provided herein are engineered viral vectors comprising a modified envelope protein. In some embodiments, the engineered viral vector comprises a first payload vector encoding a chimeric antigen receptor (CAR). In some embodiments, the modified envelope protein comprises a targeting moiety. In some embodiments, the modified envelope protein increases transduction specificity of the engineered viral vector to a target cell compared to an otherwise identical viral vector without the modified envelope protein. In some embodiments, the modified envelope protein comprises a recombinant viral envelope protein derived from an RNA virus. In some embodiments, the RNA virus comprises a Sindbis virus. In some embodiments, the modified envelope protein derived from the Sindbis virus comprises an E3 domain, an E2 domain, a 6K domain, an E1 domain, or a combination thereof. In some embodiments, the targeting moiety is located within the E2 domain. In some embodiments, the targeting moiety comprises a single-chain variable fragment (scFv), a diabody, a single variable domain on a heavy chain (VHH), a ligand that binds to a receptor, or a combination thereof. In some embodiments, the targeting moiety comprises a scFv. In some embodiments, the scFv comprises at least one base-linker, a heavy chain of the variable fragment (VH), a mid-linker, and a light chain of the variable fragment (VL). In some embodiments, the scFv is encoded by a sequence with at least 70%, at least 75%, at least 80%, at least 85%, at least 90%, at least 95%, or at least 99% sequence identity to SEQ ID NO: 1. In some embodiments, the scFv is encoded by SEQ ID NO: 1. In some embodiments, the at least one base-linker is encoded by a sequence with at least 70%, at least 75%, at least 80%, at least 85%, at least 90%, at least 95%, or at least 99% sequence identity to one of SEQ ID NOs: 7-10. In some embodiments, the at least one base-linker is encoded by one of SEQ ID NOs: 7-10. In some embodiments, the mid-linker is encoded by a sequence with at least 70%, at least 75%, at least 80%, at least 85%, at least 90%, at least 95%, or at least 99% sequence identity to one of SEQ ID NOs: 11, 18, or 19. In some embodiments, the mid-linker is encoded by one of SEQ ID NOs: 11, 18, or 19. In some embodiments, the mid-linker is encoded by a sequence with at least 70%, at least 75%, at least 80%, at least 85%, at least 90%, at least 95%, or at least 99% sequence identity to SEQ ID NO: 11. In some embodiments, the mid-linker is encoded by SEQ ID NO: 11. In some embodiments, the targeting moiety comprises a diabody. In some embodiments, the targeting moiety binds to a target ligand of the target cell. In some embodiments, the target cell is an immune cell. In some embodiments, the immune cell is a T cell, a B cell, a macrophage, a natural killer (NK) cell, a dendritic cell, or a combination thereof. In some embodiments, the T cell is a CD4+ T cell, a CD8+ T cell, a naïve T cell, a memory T cell, a γδ T cell, an exhausted T cell, or a combination thereof. In some embodiments, the target ligand of the target cell comprises a cell surface marker. In some embodiments, the target ligand of the target cell comprises an immune checkpoint protein, an immune checkpoint receptor, or a combination thereof.

In another aspect, provided herein are recombinant viruses comprising any engineered retroviral vector provided herein.

In another aspect, provided herein are cells comprising any engineered viral vector provided herein or any recombinant virus provided herein.

In another aspect, provided herein are pharmaceutical compositions comprising any engineered viral vector provided herein, any recombinant virus provided herein, or any cell provided herein. In some embodiments, the pharmaceutical composition comprises at least one additional active ingredient. In some embodiments, the pharmaceutical composition comprises at least one pharmaceutically acceptable excipient.

In another aspect, provided herein are methods of expressing a chimeric antigen receptor (CAR) in a cell of a subject comprising contacting the cell of the subject with any engineered viral vector provided herein, any recombinant virus provided herein, any cell provided herein, or any pharmaceutical composition provided herein, under conditions suitable to express the CAR in the cell of the subject.

A method of treating or preventing a disease or condition in a subject comprising administering to the subject any engineered viral vector provided herein, any recombinant virus provided herein, any cell provided herein, or any pharmaceutical composition provided herein, wherein expression of the CAR affects the disease or condition in the subject. In some embodiments, the disease or condition comprises a cancer, an infectious disease, an inflammatory disease, or an autoimmune disease.

In an aspect, provided herein are engineered viral vectors comprising a first payload vector encoding a chimeric antigen receptor (CAR) and a nucleic acid sequence encoding a deleted integrase or a defective integrase. In some embodiments, wherein the deleted integrase does not have retroviral integration activity. In some embodiments, the defective integrase has decreased retroviral integration activity compared to a wild-type integrase. In some embodiments, the deleted integrase is a deletion of about 12%, about 15%, about 20%, about 30%, about 40%, about 50%, about 60%, about 70%, about 80%, about 90%, about 95%, about 99%, or about 100% of the integrase coding sequence. In some embodiments, the deleted integrase is a deletion of at least about 80, about 160, about 240, about 320, or about 408 residues of amino acids 1331-1738 of a murine leukemia virus Gag-Pol polyprotein.

In some embodiments, the engineered viral vector comprises a nucleic acid sequence encoding a deleted integrase or a defective integrase. In some embodiments, the deleted integrase does not have retroviral integration activity. In some embodiments, the defective integrase has decreased retroviral integration activity compared to a wild-type integrase. In some embodiments, the deleted integrase is a deletion of about 12%, about 15%, about 20%, about 30%, about 40%, about 50%, about 60%, about 70%, about 80%, about 90%, about 95%, about 99%, or about 100% of the integrase coding sequence. In some embodiments, the deleted integrase is a deletion of at least about 80, about 160, about 240, about 320, or about 408 residues of amino acids 1331-1738 of a murine leukemia virus Gag-Pol polyprotein.

Additional aspects and advantages of the present disclosure will become readily apparent to those skilled in this art from the following detailed description, wherein only illustrative embodiments of the present disclosure are shown and described. As will be realized, the present disclosure is capable of other and different embodiments, and its several details are capable of modifications in various obvious respects, all without departing from the disclosure. Accordingly, the drawings and description are to be regarded as illustrative in nature, and not as restrictive.

### INCORPORATION BY REFERENCE

All publications, patents, and patent applications mentioned in this specification are herein incorporated by reference to the same extent as if each individual publication, patent, or patent application was specifically and individually indicated to be incorporated by reference. To the extent publications and patents or patent applications incorporated by reference contradict the disclosure contained in the specification, the specification is intended to supersede and/or take precedence over any such contradictory material.

### BRIEF DESCRIPTION OF THE DRAWINGS

Various features of the disclosure are set forth with particularity in the appended claims. A better understanding of the features and advantages of the present disclosure will be obtained by reference to the following detailed description that sets forth illustrative embodiments, in which the principles of the disclosure are utilized, and the accompanying drawings of which:

FIG. 1 depicts schematic of modified Sindbis virus envelope protein that may be used to direct the retroviral vectors described herein to target a certain population of immune cells. The targeting motif depicted in this figure is a single-chain variable fragment (scFv).

FIG. 2 shows the percentage of copGFP-positive SUP-T1 (CD8⁺) cells measured by flow cytometry. The cells were transduced at multiplicity of infection (MOI) of 0, 175, 350, 700, 1750, or 3500.

FIG. 3 shows the percentage of copGFP-positive Jurkat (CD8⁺) cells measured by flow cytometry. The cells were transduced at multiplicity of infection (MOI) of 0, 175, 350, 700, 1750, or 3500.

FIG. 4 shows a diagram depicting the domains of an example chimeric antigen receptor (CAR) provided herein.

FIGS. 5A-5D show the percentage of CAR-expressing human peripheral blood mononuclear cells (PBMCs) following transduction with vectors expressing CAR0, CAR1, or CAR2, with or without HSV-TK (TK). As a negative control (NC), some vectors did not express CAR. Results are shown in CD3+ CAR+ PBMCs (FIG. 5A), CD4+ CAR+ PBMCs (FIG. 5B), CD8+ CAR+ PBMCs (FIG. 5C), and CD56+ CAR+ PBMCs (FIG. 5D). “Stim” denotes stimulation with an antibody mixture of CD3/CD28, and “Stim Beads” denotes stimulation with removable beads coated with CD3/CD28 antibodies.

FIGS. 6A-6E show results from a FACS experiment demonstrating example TK expression levels across different CAR-TK constructs. FIG. 6A shows TK expression in PBMCs transduction with CAR0-TK. FIG. 6B shows TK expression in PBMCs transduction with CAR1-TK. FIG. 6C shows TK expression in PBMCs transduction with CAR2-TK. FIG. 6D shows TK expression in PBMCs transduction with a GEN2-type retrovector comprising a wild-type 4070A strain Moloney MLV envelope protein. FIG. 6E shows TK expression in PBMCs transduced with no construct (NC).

FIGS. 7A-7C demonstrate the results of an example reporter gene assay in NAML6-luc B cells. The NAML6-luc B cells were cultured with one of several groups of “effector cells”: NALM6-luc B cells (NALM6), PBMCs transduced with a vector expressing CAR0 (PBMC-CAR0), PBMCs transduced with a vector expressing CAR1 (PBMC-CAR1), PBMCs transduced with a vector expressing CAR2 (PBMC-CAR2), or untransduced PBMCs (PBMC). The assay can demonstrate the magnitude of cytotoxicity of PBMCs expressing each CAR construct against the target cell line (NAML6-luc B cells), wherein the level of relative luciferase activity is inversely proportional to cytotoxicity. 1:1, 4:1, or 9:1 ratios of effector to target cells (E:T) were tested. FIG. 7A shows relative luciferase activity after 20 hours. FIG. 7B shows relative luciferase activity after 40 hours. FIG. 7C shows relative luciferase activity after 64 hours.

FIGS. 8A-8C demonstrate the results of an example reporter gene assay. NAML6-luc B cells were cultured with one of several groups of “effector cells”: NALM6-luc B cells (NALM6), PBMCs transduced with a vector expressing CAR0 (PBMC-CAR0), PBMCs transduced with a vector expressing CAR1 (PBMC-CAR1), PBMCs transduced with a vector expressing CAR2 (PBMC-CAR2), or untransduced PBMCs (PBMC). The assay can demonstrate the magnitude of cytotoxicity of PBMCs expressing each CAR construct against the target cell line (NAML6-luc B cells), wherein the number of relative light units (RLUs) is inversely proportional to cytotoxicity. 1:1, 4:1, or 9:1 ratios of effector to target cells (E:T) were tested. FIG. 8A shows RLUs after 20 hours. FIG. 8B shows RLUs after 40 hours. FIG. 8C shows RLUs after 64 hours.

FIGS. 9A-9C demonstrate the results of an example reporter gene assay. NAML6-luc B cells were cultured with one of several groups of “effector cells”: PBMCs transduced with a vector expressing CAR0 (PBMC-CAR0), PBMCs transduced with a vector expressing CAR1 (PBMC-CAR1), PBMCs transduced with a vector expressing CAR2 (PBMC-CAR2), or untransduced PBMCs (PBMC). The assay can demonstrate the magnitude of cytotoxicity of PBMCs expressing each CAR construct against the target cell line (NAML6-luc B cells), wherein the level of relative luciferase activity is inversely proportional to cytotoxicity. 1:1, 4:1, or 9:1 ratios of effector to target cells (E:T) were tested. Relative luciferase values were standardized such that the relative luciferase activity of NAML6-luc B cells cultured with untransduced PBMCs was set to 1.0. FIG. 9A shows relative luciferase activity after 20 hours. FIG. 9B shows relative luciferase activity after 40 hours. FIG. 9C shows relative luciferase activity after 64 hours.

FIGS. 10A-10C demonstrate the results of an example reporter gene assay. Raji-eGFP cells were cultured with one of several groups of “effector cells”: Raji-eGFP cells (Raji), PBMCs transduced with a vector expressing CAR0 (PBMC-CAR0), PBMCs transduced with a vector expressing CAR1 (PBMC-CAR1), PBMCs transduced with a vector expressing CAR2 (PBMC-CAR2), or untransduced PBMCs (PBMC). The assay can demonstrate the magnitude of cytotoxicity of PBMCs expressing each CAR construct against the target cell line (Raji-eGFP cells), wherein the level of relative GFP activity is inversely proportional to cytotoxicity. 1:1, 4:1, or 9:1 ratios of effector to target cells (E:T) were tested. FIG. 10A shows relative GFP activity after 20 hours. FIG. 10B shows relative GFP activity after 40 hours. FIG. 10C shows relative GFP activity after 64 hours.

FIGS. 11A-11C demonstrate the results of an example reporter gene assay. Raji-eGFP cells were cultured with one of several groups of “effector cells”: Raji-eGFP cells (Raji), PBMCs transduced with a vector expressing CAR0 (PBMC-CAR0), PBMCs transduced with a vector expressing CAR1 (PBMC-CAR1), PBMCs transduced with a vector expressing CAR2 (PBMC-CAR2), or untransduced PBMCs (PBMC). The assay can demonstrate the magnitude of cytotoxicity of PBMCs expressing each CAR construct against the target cell line (Raji-eGFP cells), wherein the percentage of cells expressing CD19 and GFP is inversely proportional to cytotoxicity. 1:0, 1:1, 4:1, or 9:1 ratios of effector to target cells (E:T) were tested. FIG. 11A shows the percentage of CD19 and GFP expression after 20 hours. FIG. 11B shows the percentage of CD19 and GFP expression after 40 hours. FIG. 11C shows the percentage of CD19 and GFP expression after 64 hours.

FIG. 12 shows a diagram illustrating example 1^{st}, 2^{nd} 3^{rd} and 4^{th} generation CAR vectors.

FIG. 13 shows a diagram illustrating example envelope and payload structures and configurations for a CAR vector.

FIGS. 14A-14C show transduction efficiency, measured as the percentage of CAR-expressing CD3 cells (FIG. 14A), CD4 cells (FIG. 14B), or CD8 cells (FIG. 14C), in PBMCs from human Donors 1 and 2. GVO-CAR denotes a vector comprising LTR-CAR2-sv40-HSV-TK-LTR.

FIGS. 15A-15E show the magnitude of B cell depletion in human PBMC cultures with or without transduction with GVO-CAR (LTR-CAR2-sv40-HSV-TK-LTR). FIG. 15A shows results of a flow cytometry the number of B cells in untransduced Donor 1 PBMCs. FIG. 15B shows results of a flow cytometry the number of B cells in untransduced Donor 2 PBMCs. FIG. 15C shows results of a flow cytometry the number of B cells in Donor 1 PBMCs transduced with GVO-CAR. FIG. 15D shows results of a flow cytometry the number of B cells in Donor 2 PBMCs transduced with GVO-CAR. FIG. 15E shows the percentage of B cells amongst the total pool of PBMCs in Donors 1 or 2 following either no transduction, or transduction with GVO-CAR.

FIG. 16 shows relative luciferase activity as a function of the ratio of effector cells (PBMCs) to target cells (NALM6-luc). Triangles denote samples where the PBMCs were untransduced. Diamonds denote samples where the PBMCs were transduced with a vector expressing CD19-CAR.

FIGS. 17A-17B illustrate example effects of further encoding IL-12 in a CAR-encoding vector on the durability of CAR vector expression and the magnitude of cytotoxic activity against target cells. FIG. 17A shows the magnitude of CAR vector expression in CD3+ T cells transduced with a vector comprising CAR2 p2a TK (CAR), a vector comprising CAR2 p2a TK p2a IL12 (p40 t2a p35) (IL-12 Armored CAR 1), a vector comprising CAR2 p2a TK p2a IL12 (p40 tg4sx3 p35) (IL-12 Armored CAR 2), or a vector comprising CAR2 p2a TK SV40 IL12 (p40 t2a p35) (IL-12 Armored CAR 3), or CD3+ T cells which were not transduced (NC PBMC). Results are shown 3 days (D3), 6 days (D6), or 10 days (D10) post-transduction. FIG. 17B shows the results of an example reporter gene assay. The assay can demonstrate the magnitude of cytotoxicity of the effector cells, PBMCs, expressing each CAR construct against the target cell line, wherein the level of relative luciferase activity is inversely proportional to cytotoxicity. The effector cells, PBMCs, were transduced with a vector comprising CAR2 p2a TK (CAR), a vector comprising CAR2 p2a TK p2a IL12 (p40 t2a p35) (IL-12 Armored CAR 1), a vector comprising CAR2 p2a TK p2a IL12 (p40 tg4sx3 p35) (IL-12 Armored CAR 2), or a vector comprising CAR2 p2a TK SV40 IL12 (p40 t2a p35) (IL-12 Armored CAR 3), or, alternatively, they were not transduced (NC PBMC). Results are shown 3 days (D3), 6 days (D6), or 10 days (D10) post-transduction.

FIGS. 18A-18C show the results of an example reporter gene assay. The assay can demonstrate the magnitude of cytotoxicity of the effector cells, PBMCs, expressing each CAR construct against the target cell line, wherein the level of relative luciferase activity is inversely proportional to cytotoxicity. The effector cells, PBMCs, were transduced with a vector comprising CAR2 p2a TK (CAR), a vector comprising CAR2 p2a TK p2a IL12 (p40 t2a p35) (IL-12 Armored CAR 1), a vector comprising CAR2 p2a TK p2a IL12 (p40 tg4sx3 p35) (IL-12 Armored CAR 2), or a vector comprising CAR2 p2a TK SV40 IL12 (p40 t2a p35) (IL-12 Armored CAR 3), or, alternatively, they were not transduced (NC PBMC). Effector to target cell ratios of 0.02:1, 0.1:1, 0.5:1, and 1:1 were tested. FIG. 18A shows relative luciferase activity 3 days post-transduction.

FIG. 18B shows relative luciferase activity 6 days post-transduction. FIG. 18C shows relative luciferase activity 10 days post-transduction.

FIGS. 19A-19B provide a schematic of the schedule for in vivo CAR vector evaluations (FIG. 19A) and the gating strategy for analysis by flow cytometry of blood samples collected in the in vivo evaluations (FIG. 19B).

FIGS. 20A-20D show the expression of each construct in CD3+ (FIG. 20A), CD4+ (FIG. 20B), CD8+ (FIG. 20C), or NK+ cells (FIG. 20D) from C57BL/6 mouse submandibular blood samples. The mice were intravenously dosed with 100 μL of each construct, or a vehicle control, on days 1, 3, and 5. A submandibular blood sample was collected on day 11.

FIG. 21A shows the percentage of B cells amongst immune cells from C57BL/6 mouse blood samples. The mice were intravenously dosed with 100 μL of each construct, or a vehicle control, on days 1, 3, and 5. A terminal blood sample was collected on day 11. FIG. 21A shows the percentage of B cells (i.e., CD3− CD19+ cells) of parent immune cells.

FIGS. 21B-21C show the percentage of B cells amongst immune cells from C57BL/6 mouse bone marrow and liver samples. The mice were intravenously dosed with 100 μL of each construct, or a vehicle control, on days 1, 3, and 5. Bone marrow was collected on day 11. FIG. 21B shows the percentage of B cells (i.e., CD3− CD19+ cells) of parent immune cells from the bone marrow. FIG. 21C shows the percentage of B cells (i.e., CD3− CD19+ cells) of parent immune cells from the liver.

FIGS. 22A-22D shows the magnitude of PD-1 expression in T cells (i.e., CD3+ cells) from C57BL/6 mouse blood (FIG. 22A), spleen (FIG. 22B), bone marrow (FIG. 22C), and liver (FIG. 22D) samples. The mice were intravenously dosed with 100 μL of each construct, or a vehicle control, on days 1, 3, and 5. Blood, spleens, bone marrow, and livers were collected on day 11.

FIGS. 23A-23B show spleen (FIG. 23A) or liver (FIG. 23B) weight (mg) from C57BL/6 mice. The mice were intravenously dosed with 100 μL of each construct, or a vehicle control, on days 1, 3, and 5. Spleens and livers were collected on day 11.

FIGS. 24A-24C show results from a FACS experiment demonstrating example CD19-CAR expression in SUP-T1 cells, a CD8-positive cell line, which were either untransduced (cells only) (FIG. 24A), transduced with an SB-pseudotyped MLV CAR-TK vector with scFv-CD8-targeted envelope (CD19-CAR) (FIG. 24B), and transduced with an SB-pseudotyped MLV CAR-TK vector with scFv-CD8-targeted envelope (v-eTK) (FIG. 24C). An MOI of 185 was used for transduction.

FIGS. 25A-25D show results from a FACS experiment demonstrating example CD19-CAR expression in Jurkat cells, a CD3-positive cell line, which were transduced with ascending MOIs of SB-pseudotyped MLV CAR vector with scFv-CD3-targeted envelope.

FIG. 25A shows CAR expression in Jurkat cells transduced with an MOI of 16. FIG. 25B shows CAR expression in Jurkat cells transduced with an MOI of 33. FIG. 25C shows CAR expression in Jurkat cells transduced with an MOI of 65. FIG. 25D shows CAR expression in Jurkat cells transduced with an MOI of 130.

FIG. 26 shows the magnitude of CD19-CAR expression (i.e., +CD19-PE (%)) in Jurkat cells following transduction with ascending MOIs of SB-pseudotyped MLV CAR vector with scFv-CD3-targeted envelope. MOIs of 4.1, 8.1, 16.3, 32.5, 65, 130.1, and 203.3 were used for transduction.

FIGS. 27A-27D show results from a FACS experiment demonstrating example CD19-CAR expression in Jurkat cells, a CD3-positive cell line, which were transduced with ascending MOIs of SB-pseudotyped MLV CAR vector with scFv-CD3-targeted envelope. FIG. 27A shows CAR expression in Jurkat cells transduced with an MOI of 4.1. FIG. 27B shows CAR expression in Jurkat cells transduced with an MOI of 8.1. FIG. 27C shows CAR expression in Jurkat cells transduced with an MOI of 16.3. FIG. 27D shows CAR expression in Jurkat cells transduced with an MOI of 203.3.

### DETAILED DESCRIPTION

The engineered viral vectors provided herein may comprise a modified envelope protein. In some aspects, the modified envelope protein comprises a targeting moiety. The engineered viral vectors provided herein may comprise a first payload vector encoding a chimeric antigen receptor (CAR). The engineered viral vectors may further comprise other modifications.

The vectors and methods provided herein may facilitate precise and/or effective targeting of immune cells. The engineered cells may express a payload, such as a CAR. The engineered cells may provide enhanced immune cell mediated killing of cancer cells expressing an antigen of interest. The vectors and methods provided herein may prevent off-target gene delivery, e.g. a payload gene encoding CAR. The vectors provided herein may be less toxic compared to nanoparticle-based gene delivery, e.g. cationic polymers. The vectors provided herein may be less immunogenic, compared to other vectors, e.g. lentiviral vectors or adeno-associated viruses (AAVs). The vectors may allow for repeated dose injections.

### Viral Vector

In some embodiments, the engineered viral vector comprises an engineered lentiviral vector. In some embodiments, the engineered viral vector comprises an engineered retroviral vector. In some embodiments, the engineered viral vector is derived from lentivirus, poxviruses, herpes simplex virus I, adenoviruses, adeno-associated viruses, gammaretrovirus, or Sindbis viruses. In some embodiments, the engineered viral vector comprises an engineered murine leukemia gammaretroviral (MLV) vector (e.g., a Moloney, Raucher, Abelson, or Friend MLV). In some embodiments, the engineered viral vector is amphotropic. In some embodiments, the vector comprises at least one promoter for expressing the at least one polynucleotide. In some embodiments, the vector (e.g., a recombinant retroviral vector described herein) comprises at least one modified untranslated region (UTR). In some embodiments, the recombinant retroviral vector comprises at least one modification, where the recombinant retroviral vector can encode at least one amino acid mutation for increasing or modifying the targeting efficiency of the virus to a cell type. In some embodiments, the recombinant retroviral vector encodes at least one envelope protein (e.g., a wild-type or a modified envelope protein). The envelope protein can increase or modify the targeting efficiency of the virus to a cell type. For example, a 4070A MLV envelope protein can be replaced with a Sindbis-based envelope protein to direct the vector binding away from the 4070A receptor (PiT-2) towards a new target (e.g., CD3 or CD8).

In some embodiments, the modified envelope protein comprises a recombinant viral envelope protein derived from an RNA virus. In some embodiments, the modified envelope protein comprises a recombinant viral envelope protein derived from an DNA virus. In some embodiments, the RNA virus is a Sindbis virus. In some embodiments, the RNA virus is a murine leukemia virus (MLV). In some embodiments, the RNA virus is a vesicular stomatitis virus, a Rabies virus, a human immunodeficiency virus, a gammaretrovirus, or an influenza virus.

In some embodiments, the modified envelope protein derived from the Sindbis virus comprises an E3 domain. In some embodiments, the modified envelope protein derived from the Sindbis virus comprises an E2 domain. In some embodiments, the modified envelope protein derived from the Sindbis virus comprises a 6K domain. In some embodiments, the modified envelope protein derived from the Sindbis virus comprises an E1 domain. In some embodiments, the modified envelope protein derived from the Sindbis virus comprises a combination of an E3 domain, an E2 domain, a 6K domain, and an E1 domain. In some embodiments, the modified envelope protein comprises an amino acid with at least 70%, at least 75%, at least 80%, at least 85%, at least 90%, or at least 95% sequence identity with SEQ ID NO: 1. In some embodiments, the modified envelope protein comprises SEQ ID NO: 1.

In some embodiments, the targeting moiety of the modified envelope protein is located within the E1 domain. In some embodiments, the targeting moiety of the modified envelope protein is located within the E2 domain. In some embodiments, the targeting moiety of the modified envelope protein is located within the E3 domain. In some embodiments, the targeting moiety of the modified envelope protein is located within the 6K domain.

In some embodiments, the viral vector comprises an envelope protein derived from an MLV. The envelope protein can comprise an envelope protein (e.g., a wild-type or a modified envelope protein) derived from a Moloney, Rauscher, Abelson, or Friend MLV. In some embodiments, the envelope protein (e.g., a wild-type or a modified envelope protein) is derived from a Moloney MLV. The envelope protein can be a wild-type envelope protein or a modified envelope protein. In some embodiments, the envelope protein is a wild-type envelope protein. In some embodiments, the envelope protein is a modified envelope protein. In some embodiments, the viral vector comprises a wild-type 4070A Moloney murine leukemia gammaretrovirus envelope protein. In some embodiments, the 4070A Moloney murine leukemia virus envelope protein comprises an amino acid sequence with at least 70%, at least 75%, at least 80%, at least 85%, at least 90%, or at least 95% sequence identity with SEQ ID NO: 34. In some embodiments, the 4070A Moloney murine leukemia virus envelope protein comprises an amino acid sequence comprising SEQ ID NO: 34. In some embodiments, the 4070A Moloney murine leukemia virus (MoMLV) envelope protein is amphotropic. The amphotropic envelope protein can bind to an amphotropic receptor. For example, a wild-type 4070A MoMLV envelope protein can bind to the amphotropic 4070A receptor, PiT-2. The amphotropic 4070A Moloney murine leukemia virus envelope protein can facilitate transduction into multiple cell types. The multiple cell types can comprise human or murine cells. The multiple cell types can comprise immune cells. The immune cells can comprise Basophils, Eosinophils, Neutrophils, Monocytes, Macrophages, Dendritic cells, Natural killer (NK) cells, T cells, or B cells.

In some embodiments, the targeting moiety comprises a scFv. In some embodiments, the targeting moiety comprises a diabody. In some embodiments, the targeting moiety comprises a single variable domain on a heavy chain (V_{HH}). In some embodiments, the targeting moiety comprises a ligand that binds to a receptor. In some embodiments, the diabody comprises two scFvs. In some embodiments, the diabody comprises two variable domains of the heavy (V_{H}) chain of an antibody and two variable domain of the light (V_{L}) chain of an antibody. In some embodiments, the ligand is a synthetic ligand that mimics the natural ligand of the receptor. In some embodiments, the ligand is a synthetic ligand that blocks the natural ligand of the receptor. In some embodiments, the ligand is an naturally occurring ligand. In some embodiments, the ligand is an endogenous ligand. In some embodiments, the ligand is an antibody ligand.

In some embodiments, the scFv comprises at least one linker, a heavy chain of the variable fragment (V_{H}), and a light chain of the variable fragment (V_{L}). In some embodiments, the scFv comprises at least one base-linker, a heavy chain of the variable fragment (V_{H}), a mid-linker, and a light chain of the variable fragment (V_{L}). In some embodiments, the scFv comprises two base-linkers, a heavy chain of the variable fragment (V_{H}), a mid-linker, and a light chain of the variable fragment (V_{L}). In some embodiments, the base-linker comprises an amino acid with at least 70%, at least 75%, at least 80%, at least 85%, at least 90%, or at least 95% sequence identity with any one of SEQ ID NO: 7. In some embodiments, the base-linker comprises an amino acid with at least 70%, at least 75%, at least 80%, at least 85%, at least 90%, or at least 95% sequence identity with any one of SEQ ID NO: 8. In some embodiments, the base-linker comprises an amino acid with at least 70%, at least 75%, at least 80%, at least 85%, at least 90%, or at least 95% sequence identity with any one of SEQ ID NO: 9. In some embodiments, the base-linker comprises an amino acid with at least 70%, at least 75%, at least 80%, at least 85%, at least 90%, or at least 95% sequence identity with any one of SEQ ID NO: 10. In some embodiments, the base-linker comprises SEQ ID NO: 7. In some embodiments, the base-linker comprises SEQ ID NO: 8. In some embodiments, the base-linker comprises SEQ ID NO: 9. In some embodiments, the base-linker comprises SEQ ID NO: 10. In some embodiments, the mid-linker comprises an amino acid with at least 70%, at least 75%, at least 80%, at least 85%, at least 90%, or at least 95% sequence identity with any one of SEQ ID NOs: 11, 18, or 19. In some embodiments, the mid-linker comprises any one of SEQ ID NOs: 11, 18, or 19. In some embodiments, the mid-linker is encoded by a sequence with at least 70%, at least 75%, at least 80%, at least 85%, at least 90%, at least 95%, or at least 99% sequence identity to SEQ ID NO: 11. In some embodiments, the mid-linker comprises SEQ ID NO: 11. In some embodiments, the mid-linker comprises SEQ ID NO: 18. In some embodiments, the mid-linker comprises SEQ ID NO: 19. In some embodiments, the mid-linker is encoded by one of SEQ ID NOs: 11, 18, or 19. In some embodiments, the mid-linker is encoded by SEQ ID NO: 11. In some embodiments, the mid-linker is encoded by SEQ ID NO: 18. In some embodiments, the mid-linker is encoded by SEQ ID NO: 19.

In some embodiments, the scFv comprises an amino acid with at least 70%, at least 75%, at least 80%, at least 85%, at least 90%, or at least 95% sequence identity with any one of SEQ ID NO: 2. In some embodiments, the scFv comprises an amino acid with at least 70%, at least 75%, at least 80%, at least 85%, at least 90%, or at least 95% sequence identity with any one of SEQ ID NO: 3. In some embodiments, the scFv comprises an amino acid with at least 70%, at least 75%, at least 80%, at least 85%, at least 90%, or at least 95% sequence identity with any one of SEQ ID NO: 4. In some embodiments, the scFv comprises an amino acid with at least 70%, at least 75%, at least 80%, at least 85%, at least 90%, or at least 95% sequence identity with any one of SEQ ID NO:5. In some embodiments, the scFv comprises an amino acid with at least 70%, at least 75%, at least 80%, at least 85%, at least 90%, or at least 95% sequence identity with any one of SEQ ID NO: 6. In some embodiments, the modified envelope protein comprises SEQ ID NO: 2. In some embodiments, the modified envelope protein comprises SEQ ID NO: 3. In some embodiments, the modified envelope protein comprises SEQ ID NO: 4. In some embodiments, the modified envelope protein comprises SEQ ID NO: 5. In some embodiments, the modified envelope protein comprises SEQ ID NO: 6.

In some embodiments, the targeting moiety comprised the following formula: base-linker-V_{H}-mid-linker-V_{L}-base-linker. In some embodiments, the targeting moiety comprised the following formula: base-linker-V_{L}-mid-linker-V_{H}-base-linker. In some embodiments, the targeting moiety comprised the following formula: linker-V_{L}-linker-V_{H}-linker, wherein the linker is a base-linker or a mid-linker. In some embodiments, the targeting moiety comprised the following formula: linker-V_{H}-linker-V_{L}-linker, wherein the linker is a base-linker or a mid-linker. In some embodiments, the targeting moiety comprised the following formula: linker-V_{L}-linker-V_{H}-linker, wherein the linker is more than one linker. In some embodiments, the targeting moiety comprised the following formula: linker-V_{H}-linker-V_{L}-linker, wherein the linker is more than one linker.

In some embodiments, the engineered viral vector comprises a nucleic acid sequence encoding a wild-type integrase. In some embodiments, the engineered viral vector comprises a nucleic acid sequence encoding an integrase comprising no modifications or deletions. For example, the engineered viral vector can comprise a wild-type Gag-Pol polyprotein integrase. In some embodiments, the engineered viral vector comprises a nucleic acid sequence encoding a deleted integrase or a defective integrase.

In some embodiments, the vector comprising the integrase deletion renders the integrase dysfunctional such that it does not have retroviral integration activity. In some embodiments, the mutant integrase can no longer introduce the genome of a vector (e.g., a recombinant retroviral vector) into genome of a host cell comprising the vector. In some embodiments, the deleted integrase, compared with a wild-type integrase, is a deletion of about 12%, about 15%, about 20%, about 30%, about 40%, about 50%, about 60%, about 70%, about 80%, about 90%, about 95%, about 99%, or about 100% of the integrase coding sequence. In some embodiments, the deleted integrase is a deletion of at least about 80, about 160, about 240, about 320, or about 408 residues of amino acids 1331-1738 of a murine leukemia virus Gag-Pol polyprotein. In some embodiments, the murine leukemia virus Gag-Pol polyprotein comprises an amino acid sequence with at least about 70%, at least about 75%, at least about 80%, at least about 85%, at least about 90%, at least about 95%, or at least about 99% sequence identity with SEQ ID NO: 12. In some embodiments, the murine leukemia virus Gag-Pol polyprotein comprises SEQ ID NO: 12. In some embodiments, the murine leukemia virus Gag-Pol polyprotein comprises an amino acid sequence with at least about 70%, at least about 75%, at least about 80%, at least about 85%, at least about 90%, at least about 95%, or at least about 99% sequence identity with SEQ ID NO: 13. In some embodiments, the murine leukemia virus Gag-Pol polyprotein comprises SEQ ID NO: 13.

In some aspects, the target cell is a mammalian cell. In some aspects, the target cell is a human cell. In some aspects, the target cell is an immune cell. In some embodiments, the immune cell is a T cell, a B cell, a macrophage, a natural killer (NK) cell, a dendritic cell, or a combination thereof. In some embodiments, the T cell is a CD4+ T cell, a CD8+ T cell, a naïve T cell, a memory T cell, a γδ T cell, an exhausted T cell, or a combination thereof. In some embodiments, the T cell is a CD4+ T cell. In some embodiments, the T cell is a CD8+ T cell. In some embodiments, the T cell is a naïve T cell. In some embodiments, the T cell is a memory T cell. In some embodiments, the T cell is a γδ T cell. In some embodiments, the T cell is an exhausted T cell.

In some embodiments, the targeting moiety binds to a target ligand of the target cell. In some embodiments, the target ligand of the target cell comprises a cell surface marker. In some embodiments, the target ligand of the target cell comprises an immune checkpoint protein, an immune checkpoint receptor, or a combination thereof. In some embodiments, the immune checkpoint protein comprises PD-L1, CTLA-4, LAG-3, or a combination thereof.

In some aspects, the engineered viral vector is characterized by increased transduction specificity. In some aspects, the modified envelope protein increases transduction specificity efficiency of the engineered viral vector to a target cell compared to an otherwise identical viral vector without the modified envelope protein. In some embodiments, the modified envelope protein increases transduction specificity efficiency by at least 10%, at least 20%, at least 40%, at least 60%, at least 80%, at least 100%, at least 150%, at least 200%, at least 300%, at least 500%, at least 750%, or at least 1000% compared to an otherwise identical viral vector without the modified envelope protein. In some embodiments, the transduction specificity is measured by expression of a reporter gene.

In some embodiments, the CAR comprises an intracellular signaling domain, a transmembrane domain, and an antigen recognition moiety. In some embodiments, the antigen recognition moiety binds to at least one antigen. In some embodiments, the at least one antigen comprises any suitable antigen for use with a CAR. In some embodiments, the antigen recognition moiety binds to CD19, B-cell maturation antigen (BCMA), CD22, CD7, CD276, HER2, EGFR, Nectin-4, PMSA, or a combination thereof. In some embodiments, the antigen recognition moiety is an antigen binding portion of an antibody. In some embodiments, the antigen recognition moiety comprises at least one variable region. In some embodiments, the antigen recognition moiety comprises a heavy chain variable (V_{H}) region. In some embodiments, the antigen recognition moiety comprises a light chain variable (V_{L}) region. In some embodiments, the antigen recognition moiety comprises a complementarity determining region (CDR)1, a CDR2 and a CDR3. In some embodiments, the intracellular signaling domain is a CD3ζ intracellular T cell signaling domain and/or a 4-1BB intracellular T cell signaling domain. In some embodiments, the CAR comprises a checkpoint inhibitor. In some embodiments, the checkpoint inhibitor comprises PD-L1, CTLA-4, LAG-3, or a combination thereof. In some embodiments, the CD3ζ domain comprises one or more mutations comprising one or more tyrosine to phenylalanine substitutions. In some embodiments, the one or more tyrosine to phenylalanine substitutions are located at one or more immunoreceptor tyrosine-based activation motifs (ITAMs) of the CD3ζ domain. In some embodiments, the one or more tyrosine to phenylalanine substitutions are located at the two most C-terminal ITAM sequences of the CD3ζ domain.

In some aspects, the engineered viral vector comprises a second payload vector. The second payload vector may encode a transgenic product. In some embodiments, the transgenic product is a suicide gene. In some aspects, the thymidine kinase described herein can be a mutant thymidine kinase, where the mutant thymidine kinase comprises at least one amino acid mutation. In some aspects, the mutant thymidine kinase is a mutant Herpes Simplex Virus type 1 thymidine kinase (HSV1-TK) comprising at least one amino acid mutation compared to wild-type amino acid sequence of HSV1-TK: MASYPGHQHASAFDQAARSRGHSNRRTALRPRRQQEATEVRPEQKMPTLLRVYIDGPHGM GKTTTTQLLVALGSRDDIVYVPEPMTYWRVLGASETIANIYTTQHRLDQGEISAGDAAVVM TSAQITMGMPYAVTDAVLAPHIGGEAGSSHAPPPALTLIFDRHPIAALLCYPAARYLMGSMT PQAVLAFVALIPPTLPGTNIVLGALPEDRHIDRLAKRQRPGERLDLAMLAAIRRVYGLLANT VRYLQCGGSWREDWGQLSGTAVPPQGAEPQSNAGPRPHIGDTLFTLFRAPELLAPNGDLYN VFAWALDVLAKRLR (SEQ ID NO: 16). In some aspects, the mutant HSV1-TK comprises an amino acid sequence that is at least 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or more sequence identity to the HSV1-TK amino acid sequence (e.g., SEQ ID NO: 16). In some embodiments, the mutant HSV1-TK comprises a nuclear export sequence (NES). In some aspects, the NES comprises an amino acid sequence of LQKKLEELELDG (SEQ ID NO: 17).

Herpes viruses may be readily obtained from commercial sources such as the American Type Culture Collection (“ATCC”, Rockville, Md.). Herpes viruses may also be isolated from naturally occurring courses (e.g., an infected animal).

In some embodiments, the mutant HSV1-TK comprises at least one amino acid mutation at amino acid residue 25, 26, 32, 33, 167, 168, or a combination thereof compared to the wild-type amino acid sequence of HSV1-TK (SEQ ID NO: 16). In some embodiments, the mutation comprises substituting a wild-type amino acid with a polar, non-polar, basic or acidic amino acid. In some embodiments, the mutant HSV1-TK is mutated at amino acid residues 167, 168, or both. In one example, the sequence is mutated at amino acid residue 167. In another example, the sequence is mutated at amino acid residue 168. In another example, the sequence is mutated at amino acid residues 167 and 168. Amino acid residue 167 may be mutated to histidine, lysine, cysteine, serine, and phenylalanine. Amino acid residue 168 may be mutated to histidine, lysine, cysteine, serine, or phenylalanine. In some embodiments, the mutant HSV1-TK is mutated at amino acid residues 25 and/or 26. In amino acid residues 25 and/or 26 may be mutated to an amino acid chosen from the group consisting of: glycine, serine, and glutamate. In some embodiments, the mutant HSV1-TK is mutated at amino acid residues 32 and/or 33. Amino acid residues 32 and/or 33 may be mutated to an amino acid chosen from the group consisting of: glycine, serine, cysteine, glutamic acid, and aspartic acid. In some embodiments, the mutant HSV1-TK is mutated at amino acid residues 25, 26, 32, and/or 33. Amino acid residues 25, 26, 32, and/or 33, may be mutated to an amino acid chosen from the group consisting of: glycine, serine, cysteine, glutamic acid, and aspartic acid.

In some embodiments, the mutant HSV1-TK, compared to wild-type HSV1-TK, comprises increased enzymatic activity of converting a nucleoside agent into a cytotoxic drug. In some embodiments, the mutant HSV1-TK increases enzymatic activity of converting a nucleoside agent into a cytotoxic drug by at least 0.1 fold, 0.2 fold, 0.3 fold, 0.4 fold, 0.5 fold, 1.0 fold, 2.0 fold, 5.0 fold, 10.0 fold, or more compared to the enzymatic activity of a wild-type HSV1-TK converting the same nucleoside agent (e.g., a prodrug) into the cytotoxic drug.

In some embodiments, the mutant HSV1-TK increases bystander effect of cells capable of this phenomenon for killing the cell associated with the disease or condition. As used herein, the “bystander effect” refers to the phenomenon by which a HSV1-TK positive cell (e.g., cell contacted with vector described herein) exerts a kill effect on neighboring HSV1-TK negative cells following induction of HSV1-TK expression in the HSV1-TK positive cells. In some embodiments, the mutant HSV1-TK increases the bystander effect by at least 0.1 fold, 0.2 fold, 0.3 fold, 0.4 fold, 0.5 fold, 1.0 fold, 2.0 fold, 5.0 fold, 10.0 fold, or more compared to the bystander effect induced by a wild-type HSV1-TK positive cell.

In some embodiments, the transgenic product is a cytokine. In some embodiments, the cytokine includes an interleukin. In some embodiments, the transgenic product comprises any suitable cytokine or interleukin. In some embodiments, the cytokine comprises interleukin-2 (IL-2). In some embodiments, the cytokine comprises interleukin-7 (IL-7). In some embodiments, the cytokine comprises interleukin-12 (IL-12). In some embodiments, the cytokine comprises interleukin-15 (IL-15). In some embodiments, the cytokine comprises interleukin-18 (IL-18). In some embodiments, the cytokine comprises interleukin-21 (IL-21). In some embodiments, the cytokine comprises interferon-gamma (IFN-7). In some embodiments, the cytokine comprises tumor necrosis factor-alpha (TNF-α). In some embodiments, the cytokine comprises interleukin-6 (IL-6). In some embodiments, the cytokine comprises a combination of interleukin-2 (IL-2), interleukin-7 (IL-7), interleukin-12 (IL-12), interleukin-15 (IL-15), interleukin-18 (IL-18), interleukin-21 (IL-21), interferon-gamma (IFN-7), tumor necrosis factor-alpha (TNF-α), and interleukin-6 (IL-6).

### Pharmaceutical Composition

In some aspects, provided herein is a recombinant virus comprising the recombinant retroviral vector described herein. In some aspects, provided herein is a cell comprising the engineered viral vector described herein. In some aspects, provided herein is a pharmaceutical composition comprising the engineered viral vector described herein. In some aspects, provided herein is a pharmaceutical composition comprising the recombinant virus described herein. In some aspects, provided herein is a pharmaceutical composition comprising the cell described herein. In some embodiments, the pharmaceutical composition further comprises at least one additional active ingredient. In some embodiments, the pharmaceutical composition further comprises at least one pharmaceutically acceptable excipient. In some embodiments, the compositions described herein can be administered to a subject in need of treatment or prevention of a condition. In certain circumstances, it will be desirable to deliver the pharmaceutical composition as described herein in suitably formulation intramuscularly, intravenously, or intranasally.

### Method of Treatment

In some aspects, provided herein is a method of expressing the first payload in a cell of a subject. In some aspects, provided herein is a method of expressing the second payload in a cell of a subject. In some aspects, provided herein is a method of expressing a chimeric antigen receptor (CAR) in a cell of a subject. In some embodiments, the method comprises contacting the cell of the subject with the engineered viral vector described herein. In some embodiments, the method comprises contacting the cell of the subject with the recombinant virus described herein. In some embodiments, the method comprises contacting the cell of the subject with the pharmaceutical composition described herein. Contacting the cell may be carried out under conditions suitable to express the CAR in the cell of the subject. Contacting the cell may be carried out under conditions suitable to express the first payload and/or the second payload in the cell of the subject.

In some aspects, provided herein is a method of treating or preventing a disease or condition in a subject. In some embodiments, the method comprises administering to the subject the engineered viral vector as described herein. In some embodiments, the method comprises administering to the subject the recombinant virus as described herein. In some embodiments, the method comprises administering to the subject the cell as described herein. In some embodiments, the method comprises administering to the subject the pharmaceutical composition as described herein. In some embodiments, the method comprises expression of the first payload or the second payload. In some embodiments, the method comprises expression of the CAR. In some embodiments, expression of the CAR affects the disease or condition in the subject. In some embodiments, expression of the first payload and/or the second payload affects the disease or condition in the subject. In some embodiments, the disease or condition comprises a cancer. In some embodiments, the disease or condition comprises an infectious disease. In some embodiments, the disease or condition comprises an inflammatory disease. In some embodiments, the disease or condition comprises an autoimmune disease.

In some embodiments, the administration is intravenous, intramuscular, subcutaneous, or intradermal. Actual dosage levels of an agent of the disclosure (e.g., the vector, the cell comprising the vector, the recombinant virus encoded by the vector, or the pharmaceutical composition) can be varied so as to obtain an amount of the agent to achieve the desired therapeutic response for a particular subject, composition, and mode of administration, without being toxic to the subject (e.g., the subject for immunization or the subject for treatment). The selected dosage level may depend upon a variety of pharmacokinetic factors including the activity of the particular compositions of the present invention employed, the route of administration, the time of administration, the rate of excretion, the duration of the treatment, other drugs, compounds and/or materials used in combination with the particular compositions employed, the age, sex, weight, condition, general health and prior medical history of the patient being treated, and like factors well known in the medical arts.

### Numbered Embodiments

Also disclosed herein are the following embodiments:

1. An engineered viral vector comprising a first payload vector encoding a chimeric antigen receptor (CAR).
2. The engineered viral vector of embodiment 1, wherein the engineered viral vector comprises an engineered retroviral vector.
3. The engineered viral vector of embodiment 1 or embodiment 2, the engineered viral vector comprises a lentiviral vector.
4. The engineered viral vector of embodiment 1 or embodiment 2, wherein the engineered viral vector comprises an engineered murine leukemia gammaretroviral (MLV) vector.
5. The engineered viral vector of any one of embodiments 1, 2, or 4, wherein the engineered viral vector comprises an engineered Moloney MLV (MoMLV) vector.
6. The engineered viral vector of any one of embodiments 4 or 5, wherein the engineered MLV vector comprises a wild-type 4070A envelope protein sequence.
7. The engineered viral vector of any one of embodiments 1-6, wherein the engineered viral vector is amphotropic.
8. The engineered viral vector of any one of embodiments 1-7, wherein the CAR comprises an intracellular signaling domain, a transmembrane domain, and an antigen recognition moiety.
9. The engineered viral vector of embodiment 8, wherein the antigen recognition moiety binds to CD19, B-cell maturation antigen (BCMA), CD22, CD7, HER2, EGFR, Nectin-4, PMSA, or a combination thereof.
10. The engineered viral vector of embodiment 8 or embodiment 9, wherein the antigen recognition moiety is an antigen binding portion of an antibody.
11. The engineered viral vector of any one of embodiments 8-10, wherein the antigen recognition moiety comprises at least one variable region.
12. The engineered viral vector of any one of embodiments 1-11, wherein the CAR comprises a checkpoint inhibitor.
13. The engineered viral vector of embodiment 12, wherein the checkpoint inhibitor comprises PD-L1, CTLA-4, LAG-3, or a combination thereof.
14. The engineered viral vector of embodiments 1-13, wherein the engineered viral vector comprises a nucleic acid sequence encoding a deleted integrase or a defective integrase.
15. The engineered viral vector of embodiment 14, wherein the deleted integrase does not have retroviral integration activity.
16. The engineered viral vector of embodiment 14, wherein the defective integrase has decreased retroviral integration activity compared to a wild-type integrase.
17. The engineered viral vector of any one of embodiments 14-16, wherein the deleted integrase is a deletion of about 12%, about 15%, about 20%, about 30%, about 40%, about 50%, about 60%, about 70%, about 80%, about 90%, about 95%, about 99%, or about 100% of the integrase coding sequence.
18. The engineered viral vector of any one of embodiments 14-17, wherein the deleted integrase is a deletion of at least about 80, about 160, about 240, about 320, or about 408 residues of amino acids 1331-1738 of a murine leukemia virus Gag-Pol polyprotein.
19. The engineered viral vector of any one of embodiments 1-18, further comprising a second payload vector encoding a transgenic product.
20. The engineered viral vector of embodiment 19, wherein the transgenic product comprises a suicide gene or a cytokine.
21. The engineered viral vector of embodiment 20, wherein the suicide gene comprises HSV1-TK.
22. The engineered viral vector of embodiment 20, wherein the cytokine comprises interleukin-2 (IL-2), interleukin-7 (IL-7), interleukin-12 (IL-12), interleukin-15 (IL-15), interleukin-18 (IL-18), interleukin-21 (IL-21), interferon-gamma (IFN-7), tumor necrosis factor-alpha (TNF-α), interleukin-6 (IL-6), or a combination thereof.
23. The engineered viral vector of any one embodiments 1-22 comprising a modified envelope protein, wherein:
(a) the engineered viral vector comprises a first payload vector encoding a chimeric antigen receptor (CAR);
(b) the modified envelope protein comprises a targeting moiety; and
(c) the modified envelope protein increases transduction specificity of the engineered viral vector to a target cell compared to an otherwise identical viral vector without the modified envelope protein.
   24. The engineered viral vector of embodiment 23, wherein the modified envelope protein comprises a recombinant viral envelope protein derived from an RNA virus.
   25. The engineered viral vector of embodiment 24, wherein the RNA virus comprises a Sindbis virus.
   26. The engineered viral vector of any one of embodiments 23-25, wherein the modified envelope protein derived from the Sindbis virus comprises an E3 domain, an E2 domain, a 6K domain, an E1 domain, or a combination thereof.
   27. The engineered viral vector of any one of embodiments 23-26, wherein the targeting moiety is located within the E2 domain.
   28. The engineered viral vector of any one of embodiments 23-27, wherein the targeting moiety comprises a single-chain variable fragment (scFv), a diabody, a single variable domain on a heavy chain (V_{HH}), a ligand that binds to a receptor, or a combination thereof.
   29. The engineered viral vector of any one of embodiments 23-28, wherein the targeting moiety comprises a scFv.
   30. The engineered viral vector of embodiment 28 or 29, wherein the scFv comprises at least one base-linker, a heavy chain of the variable fragment (V_{H}), a mid-linker, and a light chain of the variable fragment (V_{L}).
   31. The engineered viral vector of any one of embodiments 28-30, wherein the scFv is encoded by a sequence with at least 70%, at least 75%, at least 80%, at least 85%, at least 90%, at least 95%, or at least 99% sequence identity to SEQ ID NO: 1.
   32. The engineered viral vector of any one of embodiments 28-31, wherein the scFv is encoded by SEQ ID NO: 1.
   33. The engineered viral vector of any one of embodiments 30-32, wherein the at least one base-linker is encoded by a sequence with at least 70%, at least 75%, at least 80%, at least 85%, at least 90%, at least 95%, or at least 99% sequence identity to one of SEQ ID NOs: 7-10.
   34. The engineered viral vector of any one of embodiments 30-33, wherein the at least one base-linker is encoded by one of SEQ ID NOs: 7-10.
   35. The engineered viral vector of any one of embodiments 30-34, wherein the mid-linker is encoded by a sequence with at least 70%, at least 75%, at least 80%, at least 85%, at least 90%, at least 95%, or at least 99% sequence identity to one of SEQ ID NOs: 11, 18, or 19.
   36. The engineered viral vector of any one of embodiments 30-35, wherein the mid-linker is encoded by one of SEQ ID NOs: 11, 18, or 19.
   37. The engineered viral vector of any one of embodiments 23-36, wherein the targeting moiety comprises a diabody.
   38. The engineered viral vector of any one of embodiments 23-37, wherein the targeting moiety binds to a target ligand of the target cell.
   39. The engineered viral vector of embodiment 38, wherein the target cell is an immune cell.
   40. The engineered viral vector of embodiment 39, wherein the immune cell is a T cell, a B cell, a macrophage, a natural killer (NK) cell, a dendritic cell, or a combination thereof.
   41. The engineered viral vector of embodiment 40, wherein the T cell is a CD4+ T cell, a CD8+ T cell, a naïve T cell, a memory T cell, a γδ T cell, an exhausted T cell, or a combination thereof.
   42. The engineered viral vector of any one of embodiments 38-41, wherein the target ligand of the target cell comprises a cell surface marker.
   43. The engineered viral vector of any one of embodiments 38-42, wherein the target ligand of the target cell comprises an immune checkpoint protein, an immune checkpoint receptor, or a combination thereof.
   44. A recombinant virus comprising the engineered retroviral vector of any one of embodiments 1-43.
   45. A cell comprising the engineered viral vector of any one of embodiments 1-40 or the recombinant virus of embodiment 44.
   46. A pharmaceutical composition comprising the engineered viral vector of any one of embodiments 1-43, the recombinant virus of embodiment 44, or the cell of embodiment 45.
   47. The pharmaceutical composition of embodiment 46, further comprising at least one additional active ingredient.
   48. The pharmaceutical composition of embodiment 46 or 47, further comprising at least one pharmaceutically acceptable excipient.
   49. A method of expressing a chimeric antigen receptor (CAR) in a cell of a subject comprising contacting the cell of the subject with the engineered viral vector of any one of embodiments 1-43, the recombinant virus of embodiment 44, the cell of embodiment 45, or the pharmaceutical composition of any one of embodiments 46-48, under conditions suitable to express the CAR in the cell of the subject.
   50. A method of treating or preventing a disease or condition in a subject comprising administering to the subject the engineered viral vector of any one of embodiments 1-43, the recombinant virus of embodiment 43, the cell of embodiment 45, or the pharmaceutical composition of any one of embodiments 46-48, wherein expression of the CAR affects the disease or condition in the subject.
   51. The method of embodiment 49 or 50, wherein the disease or condition comprises a cancer, an infectious disease, an inflammatory disease, or an autoimmune disease.

### Certain Definitions

All terms are intended to be understood as they would be understood by a person skilled in the art. Unless defined otherwise, all technical and scientific terms used herein have the same meaning as commonly understood by one of ordinary skill in the art to which the disclosure pertains.

The following definitions supplement those in the art and are directed to the current application and are not to be imputed to any related or unrelated case, e.g., to any commonly owned patent or application. Although any methods and materials similar or equivalent to those described herein can be used in the practice for testing of the present disclosure, the preferred materials and methods are described herein. Accordingly, the terminology used herein is for the purpose of describing particular embodiments only and is not intended to be limiting.

The terminology used herein is for the purpose of describing particular cases only and is not intended to be limiting. In this application, the use of the singular includes the plural unless specifically stated otherwise. As used herein, the singular forms “a”, “an” and “the” are intended to include the plural forms as well, unless the context clearly indicates otherwise.

In this application, the use of “or” means “and/or” unless stated otherwise. The terms “and/or” and “any combination thereof” and their grammatical equivalents as used herein, can be used interchangeably. These terms can convey that any combination is specifically contemplated. Solely for illustrative purposes, the following phrases “A, B, and/or C” or “A, B, C, or any combination thereof” can mean “A individually; B individually; C individually; A and B; B and C; A and C; and A, B, and C.” The term “or” can be used conjunctively or disjunctively, unless the context specifically refers to a disjunctive use.

The term “about” means a range within 10% of a given value.

As used in this specification and claim(s), the words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) are inclusive or open-ended and do not exclude additional, unrecited elements or method steps. It is contemplated that any embodiment discussed in this specification can be implemented with respect to any method or composition of the present disclosure, and vice versa. Furthermore, compositions of the present disclosure can be used to achieve methods of the present disclosure.

Reference in the specification to “some embodiments,” “an embodiment,” “one embodiment” or “other embodiments” means that a particular feature, structure, or characteristic described in connection with the embodiments is included in at least some embodiments, but not necessarily all embodiments, of the present disclosures. To facilitate an understanding of the present disclosure, a number of terms and phrases are defined below.

Ranges provided herein are understood to be shorthand for all of the values within the range. For example, a range of 1 to 50 is understood to include any number, combination of numbers, or sub-range from the group consisting of 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50, as well as all intervening decimal values between the aforementioned integers such as, for example, 1.1, 1.2, 1.3, 1.4, 1.5, 1.6, 1.7, 1.8, and 1.9. With respect to sub-ranges, “nested sub-ranges” that extend from either end point of the range are specifically contemplated. For example, a nested sub-range of an exemplary range of 1 to 50 may comprise 1 to 10, 1 to 20, 1 to 30, and 1 to 40 in one direction, or 50 to 40, 50 to 30, 50 to 20, and 50 to 10 in the other direction.

The term “pharmaceutically acceptable” refers to approved or approvable by a regulatory agency of the Federal or a state government or listed in the U.S. Pharmacopeia (U.S.P.) or other generally recognized pharmacopeia for use in animals, including humans.

The term “subject” refers to an animal which is the object of treatment, observation, or experiment. By way of example only, a subject includes, but is not limited to, a mammal, including, but not limited to, a human or a non-human mammal, such as a non-human primate, bovine, equine, canine, ovine, or feline.

The term “optional” or “optionally” denotes that a subsequently described event or circumstance can but need not occur, and that the description includes instances where the event or circumstance occurs and instances in which it does not.

Whenever the term “at least,” “greater than,” or “greater than or equal to” precedes the first numerical value in a series of two or more numerical values, the term “at least,” “greater than” or “greater than or equal to” applies to each of the numerical values in that series of numerical values. For example, greater than or equal to 1, 2, or 3 is equivalent to greater than or equal to 1, greater than or equal to 2, or greater than or equal to 3.

Whenever the term “no more than,” “less than,” or “less than or equal to” precedes the first numerical value in a series of two or more numerical values, the term “no more than,” “less than,” or “less than or equal to” applies to each of the numerical values in that series of numerical values. For example, less than or equal to 3, 2, or 1 is equivalent to less than or equal to 3, less than or equal to 2, or less than or equal to 1.

The “percent sequence identity” between a reference amino acid sequence and a query amino sequence (i.e., the amino sequence being analyzed to determine whether it is within a particular percent sequence identity with the reference amino acid sequence) is determined by optimally aligning the sequences using the Needleman-Wunsch alignment algorithm with a gap existence penalty of 11 and a gap extension penalty of 1 and comparing the sequences. The number of exact matches, divided by the total number of positions in the alignment (which corresponds with the number of amino acids in the reference sequence plus any gaps in the reference sequence when aligned with the query sequence) is determined and expressed as a percentage. This is the percent sequence identity between the query amino acid sequence and the reference amino acid sequence (i.e., percent sequence identity=(# of exact matches/(total # of positions in alignment)*100). An alignment using the Needleman-Wunsch alignment algorithm (with a gap existence penalty of 11 and a gap extension penalty of 1) can be generated using the “Global Align” BLAST program availa-ble at https://blast.ncbi.nlm.nih.gov/Blast.cgi.

The “percent sequence identity” between a reference nucleic acid sequence and a query nucleic acid sequence (i.e., the nucleic acid sequence being analyzed to determine whether it is within a particular percent sequence identity with the reference nucleic acid sequence) is determined by optimally aligning the sequences using the Needleman-Wunsch alignment algorithm (with match/mismatch scores of 2, −3, a gap existence penalty of 5, and a gap extension penalty of 2) and comparing the aligned nucleic acids. The number of exact match-es divided by the total number of nucleotides in the alignment (which corresponds with the number of nucleotides in the reference sequence plus any gaps in the reference sequence when aligned with the query sequence) is determined and expressed as a percentage. This is the percent sequence identity between the query nucleic acid sequence and the reference nucleic acid sequence (i.e., percent sequence identity=(# of exact matches)/(total # of nucleotides in the alignment)*100). An alignment using the Needleman-Wunsch alignment algorithm (with match/mismatch scores of 2, −3, a gap existence penalty of 5, and a gap ex-tension penalty of 2) can be generated using the “Global Align” BLAST program available at https://blast.ncbi.nlm.nih.gov/Blast.cgi.

TABLE 1
Amino Acid or DNA Sequences

| | SEQ | |
|---|---|---|
| Description | ID NO | Sequence |
| Modified Sindbis | 1 | MASAAPLVTAMCLLGNVSFPCDRPPTCYTREPSRALDILE |
| virus envelope | | ENVNHEAYDTLLNAILRCGSSGRSKRSVIDDFTLTSPYLGT |
| encoding CD8 scFv | | CSYCHHTVPCFSPVKIEQVWDEADDNTIRIQTSAQFGYDQ |
| targeting motif | | SGAASANKYRYMAAAAVTAAGHVGQVQLVQSGAEDKK |
| | | PGASVKVSCKASGFNIKDTYIHWVRQAPGQGLEWMGRID |
| | | PANDNTLYASKFQGRVTITADTSSNTAYMELSSLRSEDTA |
| | | VYYCGRGYGYYVFDHWGQGTTVTVSSSSGGGGSGGGGG |
| | | GSSRSSDIVMTQSPSSLSASVGDRVTITCRTSRSISQYLAWY |
| | | QEKPGKAPKLLIYSGSTLQSGVPSRFSGSGSGTDFTLTISSL |
| | | QPEDFATYYCQQHNENPLTFGQGTKVEIKGVHGAAVTTV |
| | | KEGTMDDIKISTSGPCRRLSYKGYFLLAKCPPGDSVTVSIV |
| | | SSNSATSCTLARKIKPKFVGREKYDLPPVHGKKIPCTVYDR |
| | | LAATTAGYITMHRPRPHAYTSYLEESSGKVYAKPPSGKNI |
| | | TYECKCGDYKTGTVSTRTEITGCTAIKQCVAYKSDQTKW |
| | | VFNSPDLIRHDDHTAQGKLHLPFKLIPSTCMVPVAHAPNVI |
| | | HGFKHISLQLDTDHLTLLTTRRLGANPEPTTEWIVGKTVR |
| | | NFTVDRDGLEYIWGNHEPVRVYAQESAPGDPHGWPHEIV |
| | | QHYYHRHPVYTILAVASATVAMMIGVTVAVLCACKARRE |
| | | CLTPYALAPNAVIPTSLALLCCVRSANAETFTETMSYLWS |
| | | NSQPFFWVQLCIPLAAFIVLMRCCSCCLPFLVVAGAYLAK |
| | | VDAYEHATTVPNVPQIPYKALVERAGYAPLNLEITVMSSE |
| | | VLPSTNQEYITCKFTTVVPSPKIKCCGSLECQPAAHADYTC |
| | | KVFGGVYPFMWGGAQCFCDSENSQMSEAYVELSADCAS |
| | | DHAQAIKVHTAAMKVGLRIVYGNTTSFLDVYVNGVTPGT |
| | | SKDLKVIAGPISASFTPFDHKVVIHRGLVYNYDFPEYGAM |
| | | KPGAFGDIQATSLTSKDLIASTDIRLLKPSYGNVHVPYTQA |
| | | SSGFEMWKNNSGRPLQETAPFGCKIAVNPLRAVDCSYGNI |
| | | PISIDIPNAAFIRTSDAPLVSTVKCEVSECTYSADFGGMATL |
| | | QYVSDREGQCPVHSHSSTATLQESTVHVLEKGAVTVHFST |
| | | ASPQANFIVSLCGKKTTCNAECKPPADHIVSTPHKNDQEF |
| | | QAAISKTSWSWLFALFGGASSLLIIGLMIFACSMMLTSTRR |
| CD8scFv_SL_ | 2 | AAGHVGQVQLVQSGAEDKKPGASVKVSCKASGFNIKDTY |
| Linker6 | | IHWVRQAPGQGLEWMGRIDPANDNTLYASKFQGRVTITA |
| | | DTSSNTAYMELSSLRSEDTAVYYCGRGYGYYVFDHWGQ |
| | | GTTVTVSSSSGGGGSGGGGGGSSRSSDIVMTQSPSSLSASV |
| | | GDRVTITCRTSRSISQYLAWYQEKPGKAPKLLIYSGSTLQS |
| | | GVPSRFSGSGSGTDFTLTISSLQPEDFATYYCQQHNENPLT |
| | | FGQGTKVEIKGVHGAA |
| CD3OKT3scFv_ | 3 | GGGGSGGGGSQVQLQQSGAELARPGASVKMSCKASGYTF |
| NIH_Linker5 | | TRYTMHWVKQRPGQGLEWIGYINPSRGYTNYNQKFKDK |
| | | ATLTTDKSSSTAYMQLSSLTSEDSAVYYCARYYDDHYCL |
| | | DYWGQGTTLTVSSSSGGGGSGGGGGGSSRSSQIVLTQSPA |
| | | IMSASPGEKVTMTCSASSSVSYMNWYQQKSGTSPKRWIY |
| | | DTSKLASGVPAHFRGSGSGTSYSLTISGMEAEDAATYYCQ |
| | | QWSSNPFTFGSGTKLEINRSGGGGSGGGG |
| CD3OKT3scFv_ | 4 | AAGHVGQVQLQQSGAELARPGASVKMSCKASGYTFTRY |
| NIH_Linker6 | | TMHWVKQRPGQGLEWIGYINPSRGYTNYNQKFKDKATLT |
| | | TDKSSSTAYMQLSSLTSEDSAVYYCARYYDDHYCLDYWG |
| | | QGTTLTVSSSSGGGGSGGGGGGSSRSSQIVLTQSPAIMSAS |
| | | PGEKVTMTCSASSSVSYMNWYQQKSGTSPKRWIYDTSKL |
| | | ASGVPAHFRGSGSGTSYSLTISGMEAEDAATYYCQQWSSN |
| | | PFTFGSGTKLEINRGVHGAA |
| CD3OKT3scFv_CR_ | 5 | GGGGSGGGGSQVQLQQSGAELARPGASVKMSCKASGYTF |
| Linker5 | | TRYTMHWVKQRPGQGLEWIGYINPSRGYTNYNQKFKDK |
| | | ATLTTDKSSSTAYMQLSSLTSEDSAVYYCARYYDDHYCL |
| | | DYWGQGTTLTVSSSSGGGGSGGGGGGSSRSSSIVLTQSPAI |
| | | MSASPGEKVTMTCSASSSVSYMNWYQQKSGTSPKRWIYD |
| | | TSKLASGVPAHFRGSGSGTSYSLTISGMEAEDAATYYCQQ |
| | | WSSNPFTFGSGTKLEINSGGGGSGGGG |
| CD3OKT3scFv_CR_ | 6 | AAGHVGQVQLQQSGAELARPGASVKMSCKASGYTFTRY |
| Linker6 | | TMHWVKQRPGQGLEWIGYINPSRGYTNYNQKFKDKATLT |
| | | TDKSSSTAYMQLSSLTSEDSAVYYCARYYDDHYCLDYWG |
| | | QGTTLTVSSSSGGGGSGGGGGGSSRSSQIVLTQSPAIMSAS |
| | | PGEKVTMTCSASSSVSYMNWYQQKSGTSPKRWIYDTSKL |
| | | ASGVPAHFRGSGSGTSYSLTISGMEAEDAATYYCQQWSSN |
| | | PFTFGSGTKLEINRGVHGAA |
| Baselinker 5 | 7 | GGGGSGGGGS |
| Baselinker 5 revert | 8 | SGGGGSGGGG |
| Baselinker 6 | 9 | AAGHVG |
| Baselinker 7 revert | 10 | GVHGAA |
| Mid-linker (CAR 0; | 18 | GSTSGSGKPGSGEGSTKG |
| Whitlow linker) | | |
| Mid-linker (CAR 1) | 19 | GGGGSGGGGSGGGGS |
| Mid-linker (CAR 2) | 11 | SSGGGGSGGGGGGSSRSS |
| Amino acid | 12 | MGQTVTTPLSLTLGHWKDVERIAHNQSVDVKKRRWVTF |
| sequence of wild | | CSAEWPTFNVGWPRDGTFNRDLITQVKIKVFSPGPHGHPD |
| type MLV gagpol | | QVPYIVTWEALAFDPPPWVKPFVHPKPPPPLPPSAPSLPLEP |
| (GPwt) (1738 aa) | | PRSTPPRSSLYPALTPSLGAKPKPQVLSDSGGPLIDLLTEDP |
| | | PPYRDPRPPPSDRDGNGGEATPAGEAPDPSPMASRLRGRR |
| | | EPPVADSTTSQAFPLRAGGNGQLQYWPFSSSDLYNWKNN |
| | | NPSFSEDPGKLTALIESVLITHQPTWDDCQQLLGTLLTGEE |
| | | KQRVLLEARKAVRGDDGRPTQLPNEVDAAFPLERPDWDY |
| | | TTQAGRNHLVHYRQLLLAGLQNAGRSPTNLAKVKGITQG |
| | | PNESPSAFLERLKEAYRRYTPYDPEDPGQETNVSMSFIWQS |
| | | APDIGRKLERLEDLKNKTLGDLVREAEKIFNKRETPEEREE |
| | | RIRRETEEKEERRRTEDEQKEKERDRRRHREMSKLLATVV |
| | | SGQKQDRQGGERRRSQLDRDQCAYCKEKGHWAKDCPKK |
| | | PRGPRGPRPQTSLLTLDD*GGQGQEPPPEPRITLKVGGQPV |
| | | TFLVDTGAQHSVLTQNPGPLSDKSAWVQGATGGKRYRW |
| | | TTDRKVHLATGKVTHSFLHVPDCPYPLLGRDLLTKLKAQI |
| | | HFEGSGAQVMGPMGQPLQVLTLNIEDEYRLHETSKEPDVS |
| | | LGSTWLSDFPQAWAETGGMGLAVRQAPLIIPLKATSTPVSI |
| | | KQYPMSQEARLGIKPHIQRLLDQGILVPCQSPWNTPLLPVK |
| | | KPGTNDYRPVQDLREVNKRVEDIHPTVPNPYNLLSGLPPS |
| | | HQWYTVLDLKDAFFCLRLHPTSQPLFAFEWRDPEMGISGQ |
| | | LTWTRLPQGFKNSPTLFDEALHRDLADFRIQHPDLILLQYV |
| | | DDLLLAATSELDCQQGTRALLQTLGNLGYRASAKKAQIC |
| | | QKQVKYLGYLLKEGQRWLTEARKETVMGQPTPKTPRQL |
| | | REFLGTAGFCRLWIPGFAEMAAPLYPLTKTGTLFNWGPDQ |
| | | QKAYQEIKQALLTAPALGLPDLTKPFELFVDEKQGYAKGV |
| | | LTQKLGPWRRPVAYLSKKLDPVAAGWPPCLRMVAAIAVL |
| | | TKDAGKLTMGQPLVILAPHAVEALVKQPPDRWLSNARMT |
| | | HYQALLLDTDRVQFGPVVALNPATLLPLPEEGLQHNCLDI |
| | | LAEAHGTRPDLTDQPLPDADHTWYTDGSSLLQEGQRKAG |
| | | AAVTTETEVIWAKALPAGTSAQRAELIALTQALKMAEGK |
| | | KLNVYTDSRYAFATAHIHGEIYRRRGLLTSEGKEIKNKDEI |
| | | LALLKALFLPKRLSIIHCPGHQKGHSAEARGNRMADQAAR |
| | | KAAITETPDTSTLLIENSSPYTSEHFHYTVTDIKDLTKLGAI |
| | | YDKTKKYWVYQGKPVMPDQFTFELLDFLHQLTHLSFSKM |
| | | KALLERSHSPYYMLNRDRTLKNITETCKACAQVNASKSA |
| | | VKQGTRVRGHRPGTHWEIDFTEIKPGLYGYKYLLVFIDTF |
| | | SGWIEAFPTKKETAKVVTKKLLEEIFPRFGMPQVLGTDNG |
| | | PAFVSKVSQTVADLLGIDWKLHCAYRPQSSGQVERMNRTI |
| | | KETLTKLTLATGSRDWVLLLPLALYRARNTPGPHGLTPYE |
| | | ILYGAPPPLVNFPDPDMTRVTNSPSLQAHLQALYLVQHEV |
| | | WRPLAAAYQEQLDRPVVPHPYRVGDTVWVRRHQTKNLE |
| | | PRWKGPYTVLLTTPTALKVDGIAAWIHAAHVKAADPGGG |
| | | PSSRLTWRVQRSQNPLKIRLTREAP |
| DNA sequence of | 13 | atgggccagactgttaccactcccttaagtttgaccttaggtcactggaaagatgtcga |
| wild type MLV | | gcggatcgctcacaaccagtcggtagatgtcaagaagagacgttgggttaccttctgct |
| gagpol (GPwt): | | ctgcagaatggccaacctttaacgtcggatggccgcgagacggcacctttaaccgagac |
| | | ctcatcacccaggttaagatcaaggtcttttcacctggcccgcatggacacccagacca |
| | | ggtcccctacatcgtgacctgggaagccttggcttttgacccccctccctgggtcaagc |
| | | cctttgtacaccctaagcctccgcctcctcttcctccatccgccccgtctctccccctt |
| | | gaacctcctegttegaccccgcctcgatcctccctttatccagccctcactccttctct |
| | | aggcgccaaacctaaacctcaagttctttctgacagtggggggccgctcatcgacctac |
| | | ttacagaagaccccccgccttatagggacccaagaccacccccttccgacagggacgga |
| | | aatggtggagaagcgacccctgcgggagaggcaccggacccctccccaatggcatctcg |
| | | cctacgtgggagacgggagccccctgtggccgactccactacctcgcaggcattccccc |
| | | tccgcgcaggaggaaacggacagcttcaatactggccgttctcctcttctgacctttac |
| | | aactggaaaaataataacccttctttttctgaagatccaggtaaactgacagctctgat |
| | | cgagtctgtcctcatcacccatcagcccacctgggacgactgtcagcagctgttgggga |
| | | ctctgctgaccggagaagaaaaacaacgggtgctcttagaggctagaaaggcggtgcgg |
| | | ggcgatgatgggcgccccactcaactgcccaatgaagtcgatgccgcttttcccctcga |
| | | gcgcccagactgggattacaccacccaggcaggtaggaaccacctagtccactatcgcc |
| | | agttgctcctagcgggtctccaaaacgcgggcagaagccccaccaatttggccaaggta |
| | | aaaggaataacacaagggcccaatgagtctccctcggccttcctagagagacttaagga |
| | | agcctatcgcaggtacactccttatgaccctgaggacccagggcaagaaactaatgtgt |
| | | ctatgtctttcatttggcagtctgccccagacattgggagaaagttagagaggttagaa |
| | | gatttaaaaaacaagacgcttggagatttggttagagaggcagaaaagatctttaataa |
| | | acgagaaaccccggaagaaagagaggaacgtatcaggagagaaacagaggaaaaagaag |
| | | aacgccgtaggacagaggatgagcagaaagagaaagaaagagatcgtaggagacataga |
| | | gagatgagcaagctattggccactgtcgttagtggacagaaacaggatagacagggagg |
| | | agaacgaaggaggtcccaactcgatcgcgaccagtgtgcctactgcaaagaaaaggggc |
| | | actgggctaaagattgtcccaagaaaccacgaggacctcggggaccaagaccccagacc |
| | | tccctcctgaccctagatgactagggaggtcagggtcaggagcccccccctgaacccag |
| | | gataaccctcaaagtcggggggcaacccgtcaccttcctggtagatactggggcccaac |
| | | actccgtgctgacccaaaatcctggacccctaagtgataagtctgcctgggtccaaggg |
| | | gctactggaggaaagcggtatcgctggaccacggatcgcaaagtacatctagctaccgg |
| | | taaggtcacccactctttcctccatgtaccagactgtccctatcctctgttaggaagag |
| | | atttgctgactaaactaaaagcccaaatccactttgagggatcaggagctcaggttatg |
| | | ggaccaatggggcagcccctgcaagtgttgaccctaaatatagaagatgagtatcggct |
| | | acatgagacctcaaaagagccagatgtttctctagggtccacatggctgtctgattttc |
| | | ctcaggcctgggcggaaaccgggggcatgggactggcagttcgccaagctcctctgatc |
| | | atacctctgaaagcaacctctacccccgtgtccataaaacaataccccatgtcacaaga |
| | | agccagactggggatcaagccccacatacagagactgttggaccagggaatactggtac |
| | | cctgccagtccccctggaacacgcccctgctacccgttaagaaaccagggactaatgat |
| | | tataggcctgtccaggatctgagagaagtcaacaagcgggtggaagacatccaccccac |
| | | cgtgcccaaccettacaacctcttgagegggctcccaccgtcccaccagtggtacactg |
| | | tgcttgatttaaaggatgcctttttctgcctgagactccaccccaccagtcagcctctc |
| | | ttcgcctttgagtggagagatccagagatgggaatctcaggacaattgacctggaccag |
| | | actcccacagggtttcaaaaacagtcccaccctgtttgatgaggcactgcacagagacc |
| | | tagcagacttccggatccagcacccagacttgatcctgctacagtacgtggatgactta |
| | | ctgctggccgccacttctgagctagactgccaacaaggtactegggccctgttacaaac |
| | | cctagggaacctcgggtatcgggcctcggccaagaaagcccaaatttgccagaaacagg |
| | | tcaagtatctggggtatcttctaaaagagggtcagagatggctgactgaggccagaaaa |
| | | gagactgtgatggggcagcctactccgaagacccctcgacaactaagggagttcctagg |
| | | gacggcaggcttctgtcgcctctggatccctgggtttgcagaaatggcagcccccttgt |
| | | accctctcaccaaaacggggactctgtttaattggggcccagaccaacaaaaggcctat |
| | | caagaaatcaagcaagctcttctaactgccccagccctggggttgccagatttgactaa |
| | | gccctttgaactctttgtcgacgagaagcagggctacgccaaaggtgtcctaacgcaaa |
| | | aactgggaccttggcgtcggccggtggcctacctgtccaaaaagctagacccagtagca |
| | | gctgggtggcccccttgcctacggatggtagcagccattgccgtactgacaaaggatgc |
| | | aggcaagctaaccatgggacagccactagtcattctggccccccatgcagtagaggcac |
| | | tagtcaaacaaccccccgaccgctggctttccaacgcccggatgactcactatcaggcc |
| | | ttgcttttggacacggaccgggtccagttcggaccggtggtagccctgaacccggctac |
| | | gctgctcccactgcctgaggaagggctgcaacacaactgccttgatatcctggccgaag |
| | | cccacggaacccgacccgacctaacggaccagccgctcccagacgccgaccacacctgg |
| | | tacacggatggaagcagtctcttacaagagggacagcgtaaggcgggagctgcggtgac |
| | | caccgagaccgaggtaatctgggctaaagccctgccagccgggacatccgctcagcggg |
| | | ctgaactgatagcactcacccaggccctaaagatggcagaaggtaagaagctaaatgtt |
| | | tatactgatagccgttatgcttttgctactgcccatatccatggagaaatatacagaag |
| | | gcgtgggttgctcacatcagaaggcaaagagatcaaaaataaagacgagatcttggccc |
| | | tactaaaagccctctttctgcccaaaagacttagcataatccattgtccaggacatcaa |
| | | aagggacacagcgccgaggctagaggcaaccggatggctgaccaagcggcccgaaaggc |
| | | agccatcacagagactccagacacctctaccctcctcatagaaaattcatcaccctaca |
| | | cctcagaacattttcattacacagtgactgatataaaggacctaaccaagttgggggcc |
| | | atttatgataaaacaaagaagtattgggtctaccaaggaaaacctgtgatgcctgacca |
| | | gtttacttttgaattattagactttcttcatcagctgactcacctcagcttctcaaaaa |
| | | tgaaggctctcctagagagaagccacagtccctactacatgctgaaccgggatcgaaca |
| | | ctcaaaaatatcactgagacctgcaaagcttgtgcacaagtcaacgccagcaagtctgc |
| | | cgttaaacagggaactagggtccgcgggcatcggcccggcactcattgggagatcgatt |
| | | tcaccgagataaagcccggattgtatggctataaatatcttctagtttttatagatacc |
| | | ttttctggctggatagaagccttcccaaccaagaaagaaaccgccaaggtcgtaaccaa |
| | | gaagctactagaggagatcttccccaggttcggcatgcctcaggtattgggaactgaca |
| | | atgggcctgccttcgtctccaaggtgagtcagacagtggccgatctgttggggattgat |
| | | tggaaattacattgtgcatacagaccccaaagctcaggccaggtagaaagaatgaatag |
| | | aaccatcaaggagactttaactaaattaacgcttgcaactggctctagagactgggtgc |
| | | tcctactccccttagccctgtaccgagcccgcaacacgccgggcccccatggcctcacc |
| | | ccatatgagatcttatatggggcacccccgccccttgtaaacttccctgaccctgacat |
| | | gacaagagttactaacagcccctctctccaagctcacttacaggctctctacttagtcc |
| | | agcacgaagtctggagacctctggcggcagcctaccaagaacaactggaccgaccggtg |
| | | gtacctcacccttaccgagtcggcgacacagtgtgggtccgccgacaccagactaagaa |
| | | cctagaacctcgctggaaaggaccttacacagtcctgctgaccacccccaccgccctca |
| | | aagtagacggcatcgcagcttggatacacgccgcccacgtgaaggctgccgaccccggg |
| | | ggtggaccatcctctagactgacatggcgcgttcaacgctctcaaaaccccttaaaaat |
| | | aaggttaacccgcgaggccccctaa |
| Amino acid | 14 | IENSSPYTSEHFHYTVTDIKDLTKLGAIYDKTKKYWVYQG |
| sequence of wild | | KPVMPDQFTFELLDFLHQLTHLSFSKMKALLERSHSPYYM |
| type integrase | | LNRDRTLKNITETCKACAQVNASKSAVKQGTRVRGHRPG |
| | | THWEIDFTEIKPGLYGYKYLLVFIDTFSGWIEAFPTKKETA |
| | | KVVTKKLLEEIFPRFGMPQVLGTDNGPAFVSKVSQTVADL |
| | | LGIDWKLHCAYRPQSSGQVERMNRTIKETLTKLTLATGSR |
| | | DWVLLLPLALYRARNTPGPHGLTPYEILYGAPPPLVNFPDP |
| | | DMTRVTNSPSLQAHLQALYLVQHEVWRPLAAAYQEQLD |
| | | RPVVPHPYRVGDTVWVRRHQTKNLEPRWKGPYTVLLTTP |
| | | TALKVDGIAAWIHAAHVKAADPGGGPSSRLTWRVQRSQN |
| | | PLKIRLTREAP |
| DNA sequence of | 15 | atagaaaattcatcaccctacacctcagaacattttcattacacagtgactgatataaa |
| wild type integrase | | ggacctaaccaagttgggggccatttatgataaaacaaagaagtattgggtctaccaag |
| | | gaaaacctgtgatgcctgaccagtttacttttgaattattagactttcttcatcagctg |
| | | actcacctcagcttctcaaaaatgaaggctctcctagagagaagccacagtccctacta |
| | | catgctgaaccgggatcgaacactcaaaaatatcactgagacctgcaaagcttgtgcac |
| | | aagtcaacgccagcaagtctgccgttaaacagggaactagggtccgcgggcatcggccc |
| | | ggcactcattgggagatcgatttcaccgagataaagcccggattgtatggctataaata |
| | | tcttctagtttttatagataccttttctggctggatagaagccttcccaaccaagaaag |
| | | aaaccgccaaggtcgtaaccaagaagctactagaggagatcttccccaggttcggcatg |
| | | cctcaggtattgggaactgacaatgggcctgccttcgtctccaaggtgagtcagacagt |
| | | ggccgatctgttggggattgattggaaattacattgtgcatacagaccccaaagctcag |
| | | gccaggtagaaagaatgaatagaaccatcaaggagactttaactaaattaacgcttgca |
| | | actggctctagagactgggtgctcctactccccttagccctgtaccgagcccgcaacac |
| | | gccgggcccccatggcctcaccccatatgagatcttatatggggcacccccgccccttg |
| | | taaacttccctgaccctgacatgacaagagttactaacagcccctctctccaagctcac |
| | | ttacaggctctctacttagtccagcacgaagtctggagacctctggoggcagcctacca |
| | | agaacaactggaccgaccggtggtacctcacccttaccgagtcggcgacacagtgtggg |
| | | tccgccgacaccagactaagaacctagaacctcgctggaaaggaccttacacagtcctg |
| | | ctgaccacccccaccgccctcaaagtagacggcatcgcagcttggatacacgccgccca |
| | | cgtgaaggctgccgaccccgggggtggaccatcctctagactgacatggcgcgttcaac |
| | | gctctcaaaaccccttaaaaataaggttaacccgcgaggccccc |
| Wild type HSV1-TK | 16 | MASYPGHQHASAFDQAARSRGHSNRRTALRPRRQQEATE |
| | | VRPEQKMPTLLRVYIDGPHGMGKTTTTQLLVALGSRDDIV |
| | | YVPEPMTYWRVLGASETIANIYTTQHRLDQGEISAGDAAV |
| | | VMTSAQITMGMPYAVTDAVLAPHIGGEAGSSHAPPPALTL |
| | | IFDRHPIAALLCYPAARYLMGSMTPQAVLAFVALIPPTLPG |
| | | TNIVLGALPEDRHIDRLAKRQRPGERLDLAMLAAIRRVYG |
| | | LLANTVRYLQCGGSWREDWGQLSGTAVPPQGAEPQSNA |
| | | GPRPHIGDTLFTLFRAPELLAPNGDLYNVFAWALDVLAKR |
| | | LR |
| Nuclear Export | 17 | LQKKLEELELDG |
| Sequence | | |
| CARO | 20 | MALPVTALLLPLALLLHAARPDIQMTQTTSSLSASLGDRV |
| | | TISCRASQDISKYLNWYQQKPDGTVKLLIYHTSRLHSGVPS |
| | | RFSGSGSGTDYSLTISNLEQEDIATYFCQQGNTLPYTFGGG |
| | | TKLEITGSTSGSGKPGSGEGSTKGEVKLQESGPGLVAPSQS |
| | | LSVTCTVSGVSLPDYGVSWIRQPPRKGLEWLGVIWGSETT |
| | | YYNSALKSRLTIIKDNSKSQVFLKMNSLQTDDTAIYYCAK |
| | | HYYYGGSYAMDYWGQGTSVTVSSTTTPAPRPPTPAPTIAS |
| | | QPLSLRPEACRPAAGGAVHTRGLDFACDIYIWAPLAGTCG |
| | | VLLLSLVITLYCKRGRKKLLYIFKQPFMRPVQTTQEEDGCS |
| | | CRFPEEEEGGCGLRVKFSRSADAPAYQQGQNQLYNELNL |
| | | GRREEYDVLDKRRGRDPEMGGKPRRKNPQEGLYNELQK |
| | | DKMAEAYSEIGMKGERRRGKGHDGLYQGLSTATKDTYD |
| | | ALHMQALPPR |
| CAR1 | 21 | MALPVTALLLPLALLLHAARPDIQMTQTTSSLSASLGDRV |
| | | TISCRASQDISKYLNWYQQKPDGTVKLLIYHTSRLHSGVPS |
| | | RFSGSGSGTDYSLTISNLEQEDIATYFCQQGNTLPYTFGGG |
| | | TKLEITGGGGSGGGGSGGGGSEVKLQESGPGLVAPSQSLS |
| | | VTCTVSGVSLPDYGVSWIRQPPRKGLEWLGVIWGSETTYY |
| | | NSALKSRLTIIKDNSKSQVFLKMNSLQTDDTAIYYCAKHY |
| | | YYGGSYAMDYWGQGTSVTVSSTTTPAPRPPTPAPTIASQP |
| | | LSLRPEACRPAAGGAVHTRGLDFACDIYIWAPLAGTCGVL |
| | | LLSLVITLYCKRGRKKLLYIFKQPFMRPVQTTQEEDGCSCR |
| | | FPEEEEGGCGLRVKFSRSADAPAYQQGQNQLYNELNLGR |
| | | REEYDVLDKRRGRDPEMGGKPRRKNPQEGLYNELQKDK |
| | | MAEAYSEIGMKGERRRGKGHDGLYQGLSTATKDTYDAL |
| | | HMQALPPR |
| CAR2 | 22 | MALPVTALLLPLALLLHAARPDIQMTQTTSSLSASLGDRV |
| | | TISCRASQDISKYLNWYQQKPDGTVKLLIYHTSRLHSGVPS |
| | | RFSGSGSGTDYSLTISNLEQEDIATYFCQQGNTLPYTFGGG |
| | | TKLEITSSGGGGSGGGGGGSSRSSEVKLQESGPGLVAPSQS |
| | | LSVTCTVSGVSLPDYGVSWIRQPPRKGLEWLGVIWGSETT |
| | | YYNSALKSRLTIIKDNSKSQVFLKMNSLQTDDTAIYYCAK |
| | | HYYYGGSYAMDYWGQGTSVTVSSTTTPAPRPPTPAPTIAS |
| | | QPLSLRPEACRPAAGGAVHTRGLDFACDIYIWAPLAGTCG |
| | | VLLLSLVITLYCKRGRKKLLYIFKQPFMRPVQTTQEEDGCS |
| | | CRFPEEEEGGCGLRVKFSRSADAPAYQQGQNQLYNELNL |
| | | GRREEYDVLDKRRGRDPEMGGKPRRKNPQEGLYNELQK |
| | | DKMAEAYSEIGMKGERRRGKGHDGLYQGLSTATKDTYD |
| | | ALHMQALPPR |
| P2A | 23 | ATNFSLLKQAGDVEENPGP |
| MAPKK Nuclear | 24 | LQKKLEELELDG |
| Export Sequence | | |
| (NES) | | |
| HSV-eTK (MAPKK | 25 | MALQKKLEELELDGSYPGHQHASAFDQAARSRGHSNGST |
| NES; R25G, R26S, | | ALRPGSQQEATEVRPEQKMPTLLRVYIDGPHGMGKTTTT |
| R32G, R32S, | | QLLVALGSRDDIVYVPEPMTYWRVLGASETIANIYTTQHR |
| A168H) | | LDQGEISAGDAAVVMTSAQITMGMPYAVTDAVLAPHIGG |
| | | EAGSSHAPPPALTLIFDRHPIAHLLCYPAARYLMGSMTPQA |
| | | VLAFVALIPPTLPGTNIVLGALPEDRHIDRLAKRQRPGERL |
| | | DLAMLAAIRRVYGLLANTVRYLQCGGSWREDWGQLSGT |
| | | AVPPQGAEPQSNAGPRPHIGDTLFTLFRAPELLAPNGDLYN |
| | | VFAWALDVLAKRLRSMHVFILDYDQSPAGCRDALLQLTS |
| | | GMVQTHVTTPGSIPTICDLARTFAREMGEAN |
| IL-12 p40 (Mus | 26 | MCPQKLTISWFAIVLLVSPLMAMWELEKDVYVVEVDWTP |
| musculus) | | DAPGETVNLTCDTPEEDDITWTSDQRHGVIGSGKTLTITVK |
| | | EFLDAGQYTCHKGGETLSHSHLLLHKKENGIWSTEILKNF |
| | | KNKTFLKCEAPNYSGRFTCSWLVQRNMDLKFNIKSSSSSP |
| | | DSRAVTCGMASLSAEKVTLDQRDYEKYSVSCQEDVTCPT |
| | | AEETLPIELALEARQQNKYENYSTSFFIRDIIKPDPPKNLQM |
| | | KPLKNSQVEVSWEYPDSWSTPHSYFSLKFFVRIQRKKEKM |
| | | KETEEGCNQKGAFLVEKTSTEVQCKGGNVCVQAQDRYY |
| | | NSSCSKWACVPCRVRS |
| T2A | 27 | EGRGSLLTCGDVEENPGP |
| SV40 Promoter | 28 | ggtgtggaaagtccccaggctccccagcaggcagaagtatgcaaagcatgcatctcaat |
| | | tagtcagcaaccaggtgtggaaagtccccaggctccccagcaggcagaagtatgcaaag |
| | | catgcatctcaattagtcagcaaccatagtcccgcccctaactcegcccatccegcccc |
| | | taactccgcccagttccgcccattctccgccccatggctgactaattttttttatttat |
| | | gcagaggccgaggccgcctcggcctctgagctattccagaagtagtgaggaggcttttt |
| | | tgg |
| IL 12 p35 (Mus | 29 | MCQSRYLLFLATLALINHLSLARVIPVSGPARCLSQSRNLL |
| musculus) | | KTTDDMVKTAREKLKHYSCTAEDIDHEDITRDQTSTLKTC |
| | | LPLELHKNESCLATRETSSTTRGSCLPPQKTSLMMTLCLGS |
| | | IYEDLKMYQTEFQAINAALQNHNHQQIILDKGMLVAIDEL |
| | | MQSLNHNGETLRQKPPVGEADPYRVKMKLCILLHAFSTR |
| | | VVTINRVMGYLSSA |
| Furin cleavage site | 30 | RRKR |
| IL-12 p40 (Homo | 31 | MCHQQLVISWFSLVFLASPLVAIWELKKDVYVVELDWYP |
| sapiens) | | DAPGEMVVLTCDTPEEDGITWTLDQSSEVLGSGKTLTIQV |
| | | KEFGDAGQYTCHKGGEVLSHSLLLLHKKEDGIWSTDILKD |
| | | QKEPKNKTFLRCEAKNYSGRFTCWWLTTISTDLTFSVKSS |
| | | RGSSDPQGVTCGAATLSAERVRGDNKEYEYSVECQEDSA |
| | | CPAAEESLPIEVMVDAVHKLKYENYTSSFFIRDIIKPDPPKN |
| | | LQLKPLKNSRQVEVSWEYPDTWSTPHSYFSLTFCVQVQG |
| | | KSKREKKDRVFTDKTSATVICRKNASISVRAQDRYYSSSW |
| | | SEWASVPCS |
| IL-12 p35 (Homo | 32 | MCPARSLLLVATLVLLDHLSLARNLPVATPDPGMFPCLHH |
| sapiens) | | SQNLLRAVSNMLQKARQTLEFYPCTSEEIDHEDITKDKTST |
| | | VEACLPLELTKNESCLNSRETSFITNGSCLASRKTSFMMAL |
| | | CLSSIYEDLKMYQVEFKTMNAKLLMDPKRQIFLDQNMLA |
| | | VIDELMQALNFNSETVPQKSSLEEPDFYKTKIKLCILLHAF |
| | | RIRAVTIDRVMSYLNAS |
| IL-12 p70 (p40- | 33 | MCHQQLVISWFSLVFLASPLVAIWELKKDVYVVELDWYP |
| GGGSGGGSGGGS- | | DAPGEMVVLTCDTPEEDGITWTLDQSSEVLGSGKTLTIQV |
| p35; Homo sapiens) | | KEFGDAGQYTCHKGGEVLSHSLLLLHKKEDGIWSTDILKD |
| | | QKEPKNKTFLRCEAKNYSGRFTCWWLTTISTDLTFSVKSS |
| | | RGSSDPQGVTCGAATLSAERVRGDNKEYEYSVECQEDSA |
| | | CPAAEESLPIEVMVDAVHKLKYENYTSSFFIRDIIKPDPPKN |
| | | LQLKPLKNSRQVEVSWEYPDTWSTPHSYFSLTFCVQVQG |
| | | KSKREKKDRVFTDKTSATVICRKNASISVRAQDRYYSSSW |
| | | SEWASVPCSGGGGSGGGGSGGGGSMCPARSLLLVATLVL |
| | | LDHLSLARNLPVATPDPGMFPCLHHSQNLLRAVSNMLQK |
| | | ARQTLEFYPCTSEEIDHEDITKDKTSTVEACLPLELTKNESC |
| | | LNSRETSFITNGSCLASRKTSFMMALCLSSIYEDLKMYQVE |
| | | FKTMNAKLLMDPKRQIFLDQNMLAVIDELMQALNFNSET |
| | | VPQKSSLEEPDFYKTKIKLCILLHAFRIRAVTIDRVMSYLN |
| | | AS |
| 4070A Moloney | 34 | MARSTLSKPPQDKINPWKPLIVMGVLLGVGMAESPHQVF |
| Amphotropic | | NVTWRVTNLMTGRTANATSLLGTVQDAFPKLYFDLCDLV |
| Envelope Protein | | GEEWDPSDQEPYVGYGCKYPAGRQRTRTFDFYVCPGHTV |
| gp70 | | KSGCGGPGEGYCGKWGCETTGQAYWKPTSSWDLISLKRG |
| | | NTPWDTGCSKVACGPCYDLSKVSNSFQGATRGGRCNPLV |
| | | LEFTDAGKKANWDGPKSWGLRLYRTGTDPITMFSLTRQV |
| | | LNVGPRVPIGPNPVLPDQRLPSSPIEIVPAPQPPSPLNTSYPP |
| | | STTSTPSTSPTSPSVPQPPPGTGDRLLALVKGAYQALNLTN |
| | | PDKTQECWLCLVSGPPYYEGVAVVGTYTNHSTAPANCTA |
| | | TSQHKLTLSEVTGQGLCMGAVPKTHQALCNTTQSAGSGS |
| | | YYLAAPAGTMWACSTGLTPCLSTTVLNLTTDYCVLVELW |
| | | PRVIYHSPDYMYGQLEQRTKYKREPVSLTLALLLGGLTM |
| | | GGIAAGIGTGTTALIKTQQFEQLHAAIQTDLNEVEKSITNL |
| | | EKSLTSLSEVVLQNRRGLDLLFLKEGGLCAALKEECCFYA |
| | | DHTGLVRDSMAKLRERLNQRQKLFETGQGWFEGLFNRSP |
| | | WFTTLISTIMGPLIVLLLILLFGPCILNRLVQFVKDRISVVQA |
| | | LVLTQQYHQLKPIEYEP |
| Wild-type Sindbis | 35 | MASAAPLVTAMCLLGNVSFPCDRPPTCYTREPSRALDILE |
| virus envelope | | ENVNHEAYDTLLNAILRCGSSGRSKRSVIDDFTLTSPYLGT |
| | | CSYCHHTVPCFSPVKIEQVWDEADDNTIRIQTSAQFGYDQ |
| | | SGAASANKYRYMSLKQDHTVKEGTMDDIKISTSGPCRRLS |
| | | YKGYFLLAKCPPGDSVTVSIVSSNSATSCTLARKIKPKFVG |
| | | REKYDLPPVHGKKIPCTVYDRLKETTAGYITMHRPRPHAY |
| | | TSYLEESSGKVYAKPPSGKNITYECKCGDYKTGTVSTRTEI |
| | | TGCTAIKQCVAYKSDQTKWVFNSPDLIRHDDHTAQGKLH |
| | | LPFKLIPSTCMVPVAHAPNVIHGFKHISLQLDTDHLTLLTT |
| | | RRLGANPEPTTEWIVGKTVRNFTVDRDGLEYIWGNHEPV |
| | | RVYAQESAPGDPHGWPHEIVQHYYHRHPVYTILAVASAT |
| | | VAMMIGVTVAVLCACKARRECLTPYALAPNAVIPTSLALL |
| | | CCVRSANAETFTETMSYLWSNSQPFFWVQLCIPLAAFIVL |
| | | MRCCSCCLPFLVVAGAYLAKVDAYEHATTVPNVPQIPYK |
| | | ALVERAGYAPLNLEITVMSSEVLPSTNQEYITCKFTTVVPS |
| | | PKIKCCGSLECQPAAHADYTCKVFGGVYPFMWGGAQCFC |
| | | DSENSQMSEAYVELSADCASDHAQAIKVHTAAMKVGLRI |
| | | VYGNTTSFLDVYVNGVTPGTSKDLKVIAGPISASFTPFDHK |
| | | VVIHRGLVYNYDFPEYGAMKPGAFGDIQATSLTSKDLIAS |
| | | TDIRLLKPSAKNVHVPYTQASSGFEMWKNNSGRPLQETAP |
| | | FGCKIAVNPLRAVDCSYGNIPISIDIPNAAFIRTSDAPLVSTV |
| | | KCEVSECTYSADFGGMATLQYVSDREGQCPVHSHSSTAT |
| | | LQESTVHVLEKGAVTVHFSTASPQANFIVSLCGKKTTCNA |
| | | ECKPPADHIVSTPHKNDQEFQAAISKTSWSWLFALFGGAS |
| | | SLLIIGLMIFACSMMLTSTRR |
| Odronextamab- | 36 | GGGGSGGGGSEVQLVESGGGLVQPGRSLRLSCAASGFTFD |
| based scFv-CD3 | | DYTMHWVRQAPGKGLEWVSGISWNSGSIGYADSVKGRF |
| with linkers (in | | TISRDNAKKSLYLQMNSLRAEDTALYYCAKDNSGYGHYY |
| Sindbis env) (linker | | YGMDVWGQGTTVTVASASTKGGGGGSGGGGSGGGGSEI |
| 5) | | VMTQSPATLSVSPGERATLSCRASQSVSSNLAWYQQKPG |
| | | QAPRLLIYGASTRATGIPARFSGSGSGTEFTLTISSLQSEDF |
| | | AVYYCQHYINWPLTFGGGTKVEIKSGGGGSGGGG |
| Odronextamab- | 37 | AAGHVGEVQLVESGGGLVQPGRSLRLSCAASGFTFDDYT |
| based scFv-CD3 | | MHWVRQAPGKGLEWVSGISWNSGSIGYADSVKGRFTISR |
| with linkers (in | | DNAKKSLYLQMNSLRAEDTALYYCAKDNSGYGHYYYG |
| Sindbis env) (linker | | MDVWGQGTTVTVASASTKGGGGGSGGGGSGGGGSEIVM |
| 6) | | TQSPATLSVSPGERATLSCRASQSVSSNLAWYQQKPGQAP |
| | | RLLIYGASTRATGIPARFSGSGSGTEFTLTISSLQSEDFAVY |
| | | YCQHYINWPLTFGGGTKVEIKGVHGAA |
| CAR2-P2A-vTK | 38 | MALPVTALLLPLALLLHAARPDIQMTQTTSSLSASLGDRV |
| | | TISCRASQDISKYLNWYQQKPDGTVKLLIYHTSRLHSGVPS |
| | | RFSGSGSGTDYSLTISNLEQEDIATYFCQQGNTLPYTFGGG |
| | | TKLEITSSGGGGSGGGGGGSSRSSEVKLQESGPGLVAPSQS |
| | | LSVTCTVSGVSLPDYGVSWIRQPPRKGLEWLGVIWGSETT |
| | | YYNSALKSRLTIIKDNSKSQVFLKMNSLQTDDTAIYYCAK |
| | | HYYYGGSYAMDYWGQGTSVTVSSTTTPAPRPPTPAPTIAS |
| | | QPLSLRPEACRPAAGGAVHTRGLDFACDIYIWAPLAGTCG |
| | | VLLLSLVITLYCKRGRKKLLYIFKQPFMRPVQTTQEEDGCS |
| | | CRFPEEEEGGCGLRVKFSRSADAPAYQQGQNQLYNELNL |
| | | GRREEYDVLDKRRGRDPEMGGKPRRKNPQEGLYNELQK |
| | | DKMAEAYSEIGMKGERRRGKGHDGLYQGLSTATKDTYD |
| | | ALHMQALPPRGSGATNFSLLKQAGDVEENPGPMALQKKL |
| | | EELELDGSYPGHQHASAFDQAARSRGHSNGSTALRPGSQQ |
| | | EATEVRPEQKMPTLLRVYIDGPHGMGKTTTTQLLVALGSR |
| | | DDIVYVPEPMTYWRVLGASETIANIYTTQHRLDQGEISAG |
| | | DAAVVMTSAQITMGMPYAVTDAVLAPHIGGEAGSSHAPP |
| | | PALTLIFDRHPIAHLLCYPAARYLMGSMTPQAVLAFVALIP |
| | | PTLPGTNIVLGALPEDRHIDRLAKRQRPGERLDLAMLAAIR |
| | | RVYGLLANTVRYLQCGGSWREDWGQLSGTAVPPQGAEP |
| | | QSNAGPRPHIGDTLFTLFRAPELLAPNGDLYNVFAWALDV |
| | | LAKRLRSMHVFILDYDQSPAGCRDALLQLTSGMVQTHVT |
| | | TPGSIPTICDLARTFAREMGEAN |
| CAR2-P2A-vTK | 39 | ATGGCCTTACCAGTGACCGCCTTGCTCCTGCCGCTGGCC |
| | | TTGCTGCTCCACGCCGCCAGGCCGGACATCCAGATGAC |
| | | ACAGACTACATCCTCCCTGTCTGCCTCTCTGGGAGACAG |
| | | AGTCACCATCAGTTGCAGGGCAAGTCAGGACATTAGTA |
| | | AATATTTAAATTGGTATCAGCAGAAACCAGATGGAACT |
| | | GTTAAACTCCTGATCTACCATACATCAAGATTACACTCA |
| | | GGAGTCCCATCAAGGTTCAGTGGCAGTGGGTCTGGAAC |
| | | AGATTATTCTCTCACCATTAGCAACCTGGAGCAAGAAG |
| | | ATATTGCCACTTACTTTTGCCAACAGGGTAATACGCTTC |
| | | CGTACACGTTCGGAGGGGGGACCAAGCTGGAGATCACA |
| | | TCCTCTGGCGGCGGCGGCTCTGGCGGCGGAGGAGGAGG |
| | | CAGCTCCAGGTCTAGCGAGGTGAAACTGCAGGAGTCAG |
| | | GACCTGGCCTGGTGGCGCCCTCACAGAGCCTGTCCGTC |
| | | ACATGCACTGTCTCAGGGGTCTCATTACCCGACTATGGT |
| | | GTAAGCTGGATTCGCCAGCCTCCACGAAAGGGTCTGGA |
| | | GTGGCTGGGAGTAATATGGGGTAGTGAAACCACATACT |
| | | ATAATTCAGCTCTCAAATCCAGACTGACCATCATCAAG |
| | | GACAACTCCAAGAGCCAAGTTTTCTTAAAAATGAACAG |
| | | TCTGCAAACTGATGACACAGCCATTTACTACTGTGCCAA |
| | | ACATTATTACTACGGTGGTAGCTATGCTATGGACTACTG |
| | | GGGCCAAGGAACCTCAGTCACCGTCTCCTCAACCACGA |
| | | CGCCAGCGCCGCGACCACCAACACCGGCGCCCACCATC |
| | | GCGTCGCAGCCCCTGTCCCTGCGCCCAGAGGCGTGCCG |
| | | GCCAGCGGCGGGGGGCGCAGTGCACACGAGGGGGCTG |
| | | GACTTCGCCTGTGATATCTACATCTGGGCGCCCTTGGCC |
| | | GGGACTTGTGGGGTCCTTCTCCTGTCACTGGTTATCACC |
| | | CTTTACTGCAAACGGGGCAGAAAGAAACTCCTGTATAT |
| | | ATTCAAACAACCATTTATGAGACCAGTACAAACTACTC |
| | | AAGAGGAAGATGGCTGTAGCTGCCGATTTCCAGAAGAA |
| | | GAAGAAGGAGGATGTGGACTGAGAGTGAAGTTCAGCA |
| | | GGAGCGCAGACGCCCCCGCGTACCAGCAGGGCCAGAA |
| | | CCAGCTCTATAACGAGCTCAATCTAGGACGAAGAGAGG |
| | | AGTACGATGTTTTGGACAAGAGACGTGGCCGGGACCCT |
| | | GAGATGGGGGGAAAGCCGAGAAGGAAGAACCCTCAGG |
| | | AAGGCCTGTACAATGAACTGCAGAAAGATAAGATGGCG |
| | | GAGGCCTACAGTGAGATTGGGATGAAAGGCGAGCGCC |
| | | GGAGGGGCAAGGGGCACGATGGCCTTTACCAGGGTCTC |
| | | AGTACAGCCACCAAGGACACCTACGACGCCCTTCACAT |
| | | GCAGGCCCTGCCCCCTCGCGGCTCTGGAGCAACTAACT |
| | | TTTCACTGTTGAAACAGGCCGGAGATGTAGAGGAAAAC |
| | | CCTGGCCCCATGGCCCTGCAGAAAAAGCTGGAAGAGCT |
| | | GGAACTGGATGGCAGCTACCCCGGCCACCAGCACGCCA |
| | | GCGCCTTCGACCAGGCCGCCCGCAGCCGCGGCCACAGC |
| | | AACGGCAGCACCGCACTGCGGCCAGGATCTCAGCAGGA |
| | | GGCCACCGAGGTGCGCCCCGAGCAGAAGATGCCCACCC |
| | | TGCTGCGCGTGTACATCGACGGACCACACGGCATGGGC |
| | | AAGACCACCACCACCCAGCTGCTGGTGGCCCTGGGCAG |
| | | CCGCGACGACATCGTGTACGTGCCCGAGCCCATGACCT |
| | | ACTGGCGCGTGCTGGGCGCCAGCGAGACCATCGCCAAC |
| | | ATCTACACCACCCAGCACCGCCTGGACCAAGGCGAGAT |
| | | CAGCGCCGGCGACGCCGCCGTGGTGATGACCAGCGCCC |
| | | AGATTACAATGGGCATGCCCTACGCCGTGACCGACGCC |
| | | GTGCTGGCACCACACATCGGCGGCGAGGCCGGCAGCAG |
| | | CCACGCACCACCACCAGCACTGACCCTGATCTTCGACC |
| | | GGCACCCAATCGCACACCTGCTGTGCTACCCGGCAGCA |
| | | CGCTACCTGATGGGCTCCATGACACCACAAGCCGTGCT |
| | | GGCCTTCGTGGCCCTGATCCCACCAACACTGCCCGGCA |
| | | CCAACATCGTGCTGGGCGCCCTGCCCGAGGACCGCCAC |
| | | ATCGACCGCCTGGCCAAGCGCCAGCGCCCCGGCGAGCG |
| | | CCTGGACCTGGCCATGCTGGCCGCCATCCGCCGCGTGT |
| | | ACGGCCTGCTGGCCAACACCGTGCGCTACCTGCAGTGC |
| | | GGCGGCAGCTGGCGCGAGGACTGGGGCCAGCTGAGCG |
| | | GCACCGCCGTGCCACCACAGGGCGCCGAGCCACAGAGC |
| | | AACGCCGGACCACGACCACACATCGGCGACACCCTGTT |
| | | CACCCTGTTCCGGGCACCAGAGCTGCTGGCACCAAACG |
| | | GCGACCTGTACAACGTGTTCGCCTGGGCCCTGGACGTG |
| | | CTGGCCAAGCGCCTGCGCTCCATGCACGTGTTCATCCTG |
| | | GACTACGACCAGTCACCGGCCGGCTGCCGCGACGCCCT |
| | | GCTGCAGCTGACCAGCGGCATGGTGCAGACCCACGTGA |
| | | CAACACCCGGCAGCATCCCAACAATCTGCGACCTGGCC |
| | | CGCACCTTCGCCCGCGAGATGGGCGAGGCCAACTAA |
| CAR2-P2A-vTK- | 40 | ATGGCCTTACCAGTGACCGCCTTGCTCCTGCCGCTGGCC |
| SV40-mIL12 | | TTGCTGCTCCACGCCGCCAGGCCGGACATCCAGATGAC |
| | | ACAGACTACATCCTCCCTGTCTGCCTCTCTGGGAGACAG |
| | | AGTCACCATCAGTTGCAGGGCAAGTCAGGACATTAGTA |
| | | AATATTTAAATTGGTATCAGCAGAAACCAGATGGAACT |
| | | GTTAAACTCCTGATCTACCATACATCAAGATTACACTCA |
| | | GGAGTCCCATCAAGGTTCAGTGGCAGTGGGTCTGGAAC |
| | | AGATTATTCTCTCACCATTAGCAACCTGGAGCAAGAAG |
| | | ATATTGCCACTTACTTTTGCCAACAGGGTAATACGCTTC |
| | | CGTACACGTTCGGAGGGGGGACCAAGCTGGAGATCACA |
| | | TCCTCTGGCGGCGGCGGCTCTGGCGGCGGAGGAGGAGG |
| | | CAGCTCCAGGTCTAGCGAGGTGAAACTGCAGGAGTCAG |
| | | GACCTGGCCTGGTGGCGCCCTCACAGAGCCTGTCCGTC |
| | | ACATGCACTGTCTCAGGGGTCTCATTACCCGACTATGGT |
| | | GTAAGCTGGATTCGCCAGCCTCCACGAAAGGGTCTGGA |
| | | GTGGCTGGGAGTAATATGGGGTAGTGAAACCACATACT |
| | | ATAATTCAGCTCTCAAATCCAGACTGACCATCATCAAG |
| | | GACAACTCCAAGAGCCAAGTTTTCTTAAAAATGAACAG |
| | | TCTGCAAACTGATGACACAGCCATTTACTACTGTGCCAA |
| | | ACATTATTACTACGGTGGTAGCTATGCTATGGACTACTG |
| | | GGGCCAAGGAACCTCAGTCACCGTCTCCTCAACCACGA |
| | | CGCCAGCGCCGCGACCACCAACACCGGCGCCCACCATC |
| | | GCGTCGCAGCCCCTGTCCCTGCGCCCAGAGGCGTGCCG |
| | | GCCAGCGGCGGGGGGCGCAGTGCACACGAGGGGGCTG |
| | | GACTTCGCCTGTGATATCTACATCTGGGCGCCCTTGGCC |
| | | GGGACTTGTGGGGTCCTTCTCCTGTCACTGGTTATCACC |
| | | CTTTACTGCAAACGGGGCAGAAAGAAACTCCTGTATAT |
| | | ATTCAAACAACCATTTATGAGACCAGTACAAACTACTC |
| | | AAGAGGAAGATGGCTGTAGCTGCCGATTTCCAGAAGAA |
| | | GAAGAAGGAGGATGTGGACTGAGAGTGAAGTTCAGCA |
| | | GGAGCGCAGACGCCCCCGCGTACCAGCAGGGCCAGAA |
| | | CCAGCTCTATAACGAGCTCAATCTAGGACGAAGAGAGG |
| | | AGTACGATGTTTTGGACAAGAGACGTGGCCGGGACCCT |
| | | GAGATGGGGGGAAAGCCGAGAAGGAAGAACCCTCAGG |
| | | AAGGCCTGTACAATGAACTGCAGAAAGATAAGATGGCG |
| | | GAGGCCTACAGTGAGATTGGGATGAAAGGCGAGCGCC |
| | | GGAGGGGCAAGGGGCACGATGGCCTTTACCAGGGTCTC |
| | | AGTACAGCCACCAAGGACACCTACGACGCCCTTCACAT |
| | | GCAGGCCCTGCCCCCTCGCGGCTCTGGAGCAACTAACT |
| | | TTTCACTGTTGAAACAGGCCGGAGATGTAGAGGAAAAC |
| | | CCTGGCCCCATGGCCCTGCAGAAAAAGCTGGAAGAGCT |
| | | GGAACTGGATGGCAGCTACCCCGGCCACCAGCACGCCA |
| | | GCGCCTTCGACCAGGCCGCCCGCAGCCGCGGCCACAGC |
| | | AACGGCAGCACCGCACTGCGGCCAGGATCTCAGCAGGA |
| | | GGCCACCGAGGTGCGCCCCGAGCAGAAGATGCCCACCC |
| | | TGCTGCGCGTGTACATCGACGGACCACACGGCATGGGC |
| | | AAGACCACCACCACCCAGCTGCTGGTGGCCCTGGGCAG |
| | | CCGCGACGACATCGTGTACGTGCCCGAGCCCATGACCT |
| | | ACTGGCGCGTGCTGGGCGCCAGCGAGACCATCGCCAAC |
| | | ATCTACACCACCCAGCACCGCCTGGACCAAGGCGAGAT |
| | | CAGCGCCGGCGACGCCGCCGTGGTGATGACCAGCGCCC |
| | | AGATTACAATGGGCATGCCCTACGCCGTGACCGACGCC |
| | | GTGCTGGCACCACACATCGGCGGCGAGGCCGGCAGCAG |
| | | CCACGCACCACCACCAGCACTGACCCTGATCTTCGACC |
| | | GGCACCCAATCGCACACCTGCTGTGCTACCCGGCAGCA |
| | | CGCTACCTGATGGGCTCCATGACACCACAAGCCGTGCT |
| | | GGCCTTCGTGGCCCTGATCCCACCAACACTGCCCGGCA |
| | | CCAACATCGTGCTGGGCGCCCTGCCCGAGGACCGCCAC |
| | | ATCGACCGCCTGGCCAAGCGCCAGCGCCCCGGCGAGCG |
| | | CCTGGACCTGGCCATGCTGGCCGCCATCCGCCGCGTGT |
| | | ACGGCCTGCTGGCCAACACCGTGCGCTACCTGCAGTGC |
| | | GGCGGCAGCTGGCGCGAGGACTGGGGCCAGCTGAGCG |
| | | GCACCGCCGTGCCACCACAGGGCGCCGAGCCACAGAGC |
| | | AACGCCGGACCACGACCACACATCGGCGACACCCTGTT |
| | | CACCCTGTTCCGGGCACCAGAGCTGCTGGCACCAAACG |
| | | GCGACCTGTACAACGTGTTCGCCTGGGCCCTGGACGTG |
| | | CTGGCCAAGCGCCTGCGCTCCATGCACGTGTTCATCCTG |
| | | GACTACGACCAGTCACCGGCCGGCTGCCGCGACGCCCT |
| | | GCTGCAGCTGACCAGCGGCATGGTGCAGACCCACGTGA |
| | | CAACACCCGGCAGCATCCCAACAATCTGCGACCTGGCC |
| | | CGCACCTTCGCCCGCGAGATGGGCGAGGCCAACTAAGG |
| | | ATCCCTCGAGATACACATTGTGTTCTCGACTCGCTGTGG |
| | | AATGTGTGTCAGTTAGGGTGTGGAAAGTCCCCAGGCTC |
| | | CCCAGCAGGCAGAAGTATGCAAAGCATGCATCTCAATT |
| | | AGTCAGCAACCAGGTGTGGAAAGTCCCCAGGCTCCCCA |
| | | GCAGGCAGAAGTATGCAAAGCATGCATCTCAATTAGTC |
| | | AGCAACCATAGTCCCGCCCCTAACTCCGCCCATCCCGCC |
| | | CCTAACTCCGCCCAGTTCCGCCCATTCTCCGCCCCATGG |
| | | CTGACTAATTTTTTTTATTTATGCAGAGGCCGAGGCCGC |
| | | CTCGGCCTCTGAGCTATTCCAGAAGTAGTGAGGAGGCT |
| | | TTTTTGGAGGCCTAGTCTTTTGCAAAAAGCTCGAAGATC |
| | | AATTCCGATCTGATCAACCGGTATGTGCCCCCAGAAGC |
| | | TGACCATCAGCTGGTTCGCCATCGTGCTGCTGGTGTCCC |
| | | CTCTGATGGCTATGTGGGAGCTGGAGAAGGACGTGTAC |
| | | GTGGTGGAGGTGGACTGGACACCAGATGCCCCTGGCGA |
| | | GACCGTGAACCTGACATGCGACACCCCCGAGGAGGACG |
| | | ACATCACCTGGACATCTGATCAGCGGCACGGCGTGATC |
| | | GGCAGCGGAAAGACCCTGACAATCACCGTGAAGGAGTT |
| | | TCTGGATGCTGGACAGTACACATGTCACAAGGGCGGAG |
| | | AGACCCTGTCTCACAGCCACCTGCTGCTGCACAAGAAG |
| | | GAGAACGGCATCTGGTCCACAGAGATCCTGAAGAACTT |
| | | CAAGAACAAGACCTTTCTGAAGTGCGAGGCCCCAAACT |
| | | ACAGCGGACGGTTCACCTGTTCCTGGCTGGTGCAGCGC |
| | | AACATGGACCTGAAGTTTAACATCAAGAGCAGCAGCAG |
| | | CAGCCCCGATTCTCGGGCTGTGACATGTGGCATGGCCTC |
| | | CCTGTCTGCTGAGAAGGTGACCCTGGACCAGCGCGATT |
| | | ACGAGAAGTACAGCGTGTCCTGCCAGGAGGACGTGACA |
| | | TGTCCAACCGCCGAGGAGACACTGCCAATCGAGCTGGC |
| | | CCTGGAGGCTAGGCAGCAGAACAAGTACGAGAACTACT |
| | | CTACCAGCTTCTTTATCCGCGACATCATCAAGCCTGATC |
| | | CCCCTAAGAACCTGCAGATGAAGCCACTGAAGAACTCC |
| | | CAGGTCGAGGTGTCTTGGGAGTACCCCGACTCCTGGTCT |
| | | ACACCTCACTCTTACTTCAGCCTGAAGTTCTTCGTGAGA |
| | | ATCCAGAGAAAGAAGGAGAAGATGAAGGAGACCGAGG |
| | | AGGGCTGCAACCAGAAGGGAGCTTTTCTGGTGGAGAAG |
| | | ACAAGCACCGAGGTGCAGTGCAAGGGCGGAAACGTGT |
| | | GCGTGCAGGCCCAGGACAGGTACTACAACTCTAGCTGT |
| | | TCCAAGTGGGCTTGCGTGCCTTGTCGGGTGCGCTCTAGG |
| | | AGAAAGAGGGGCAGCGGAGAGGGAAGGGGCAGCCTGC |
| | | TGACATGCGGCGATGTGGAGGAGAACCCCGGACCTATG |
| | | TGCCAGAGCAGATACCTGCTGTTCCTGGCCACCCTGGCT |
| | | CTGATCAACCACCTGTCTCTGGCCAGAGTGATCCCTGTG |
| | | AGCGGCCCAGCTAGGTGCCTGAGCCAGTCCAGAAACCT |
| | | GCTGAAGACCACAGACGATATGGTGAAGACAGCCAGG |
| | | GAGAAGCTGAAGCACTACTCCTGTACCGCTGAGGACAT |
| | | CGATCACGAGGACATCACAAGAGATCAGACATCCACCC |
| | | TGAAGACCTGCCTGCCTCTGGAGCTGCACAAGAACGAG |
| | | TCTTGTCTGGCCACAAGGGAGACCTCCTCTACCACAAG |
| | | AGGCTCCTGCCTGCCACCCCAGAAGACATCTCTGATGA |
| | | TGACCCTGTGCCTGGGAAGCATCTACGAGGACCTGAAG |
| | | ATGTACCAGACAGAGTTCCAGGCCATCAACGCCGCTCT |
| | | GCAGAACCACAACCACCAGCAGATCATCCTGGACAAGG |
| | | GCATGCTGGTGGCTATCGATGAGCTGATGCAGTCTCTG |
| | | AACCACAACGGCGAGACCCTGAGGCAGAAGCCTCCAGT |
| | | GGGAGAGGCCGATCCATACAGAGTGAAGATGAAGCTGT |
| | | GCATCCTGCTGCACGCTTTTAGCACACGGGTGGTGACC |
| | | ATCAACCGCGTGATGGGCTACCTGAGCAGCGCCTGA |
| mGEN-1013 (HSV- | 41 | ATGGCCCTGCAGAAAAAGCTGGAAGAGCTGGAACTGG |
| eTK-SV40-mIL12) | | ATGGCAGCTACCCCGGCCACCAGCACGCCAGCGCCTTC |
| | | GACCAGGCCGCCCGCAGCCGCGGCCACAGCAACGGCA |
| | | GCACCGCACTGCGGCCAGGATCTCAGCAGGAGGCCACC |
| | | GAGGTGCGCCCCGAGCAGAAGATGCCCACCCTGCTGCG |
| | | CGTGTACATCGACGGACCACACGGCATGGGCAAGACCA |
| | | CCACCACCCAGCTGCTGGTGGCCCTGGGCAGCCGCGAC |
| | | GACATCGTGTACGTGCCCGAGCCCATGACCTACTGGCG |
| | | CGTGCTGGGCGCCAGCGAGACCATCGCCAACATCTACA |
| | | CCACCCAGCACCGCCTGGACCAAGGCGAGATCAGCGCC |
| | | GGCGACGCCGCCGTGGTGATGACCAGCGCCCAGATTAC |
| | | AATGGGCATGCCCTACGCCGTGACCGACGCCGTGCTGG |
| | | CACCACACATCGGCGGCGAGGCCGGCAGCAGCCACGCA |
| | | CCACCACCAGCACTGACCCTGATCTTCGACCGGCACCC |
| | | AATCGCACACCTGCTGTGCTACCCGGCAGCACGCTACC |
| | | TGATGGGCTCCATGACACCACAAGCCGTGCTGGCCTTC |
| | | GTGGCCCTGATCCCACCAACACTGCCCGGCACCAACAT |
| | | CGTGCTGGGCGCCCTGCCCGAGGACCGCCACATCGACC |
| | | GCCTGGCCAAGCGCCAGCGCCCCGGCGAGCGCCTGGAC |
| | | CTGGCCATGCTGGCCGCCATCCGCCGCGTGTACGGCCT |
| | | GCTGGCCAACACCGTGCGCTACCTGCAGTGCGGCGGCA |
| | | GCTGGCGCGAGGACTGGGGCCAGCTGAGCGGCACCGCC |
| | | GTGCCACCACAGGGCGCCGAGCCACAGAGCAACGCCG |
| | | GACCACGACCACACATCGGCGACACCCTGTTCACCCTG |
| | | TTCCGGGCACCAGAGCTGCTGGCACCAAACGGCGACCT |
| | | GTACAACGTGTTCGCCTGGGCCCTGGACGTGCTGGCCA |
| | | AGCGCCTGCGCTCCATGCACGTGTTCATCCTGGACTACG |
| | | ACCAGTCACCGGCCGGCTGCCGCGACGCCCTGCTGCAG |
| | | CTGACCAGCGGCATGGTGCAGACCCACGTGACAACACC |
| | | CGGCAGCATCCCAACAATCTGCGACCTGGCCCGCACCT |
| | | TCGCCCGCGAGATGGGCGAGGCCAACTAAGGATCCCTC |
| | | GAGATACACATTGTGTTCTCGACTCGCTGTGGAATGTGT |
| | | GTCAGTTAGGGTGTGGAAAGTCCCCAGGCTCCCCAGCA |
| | | GGCAGAAGTATGCAAAGCATGCATCTCAATTAGTCAGC |
| | | AACCAGGTGTGGAAAGTCCCCAGGCTCCCCAGCAGGCA |
| | | GAAGTATGCAAAGCATGCATCTCAATTAGTCAGCAACC |
| | | ATAGTCCCGCCCCTAACTCCGCCCATCCCGCCCCTAACT |
| | | CCGCCCAGTTCCGCCCATTCTCCGCCCCATGGCTGACTA |
| | | ATTTTTTTTATTTATGCAGAGGCCGAGGCCGCCTCGGCC |
| | | TCTGAGCTATTCCAGAAGTAGTGAGGAGGCTTTTTTGGA |
| | | GGCCTAGTCTTTTGCAAAAAGCTCGAAGATCAATTCCG |
| | | ATCTGATCAACCGGTATGTGCCCCCAGAAGCTGACCAT |
| | | CAGCTGGTTCGCCATCGTGCTGCTGGTGTCCCCTCTGAT |
| | | GGCTATGTGGGAGCTGGAGAAGGACGTGTACGTGGTGG |
| | | AGGTGGACTGGACACCAGATGCCCCTGGCGAGACCGTG |
| | | AACCTGACATGCGACACCCCCGAGGAGGACGACATCAC |
| | | CTGGACATCTGATCAGCGGCACGGCGTGATCGGCAGCG |
| | | GAAAGACCCTGACAATCACCGTGAAGGAGTTTCTGGAT |
| | | GCTGGACAGTACACATGTCACAAGGGCGGAGAGACCCT |
| | | GTCTCACAGCCACCTGCTGCTGCACAAGAAGGAGAACG |
| | | GCATCTGGTCCACAGAGATCCTGAAGAACTTCAAGAAC |
| | | AAGACCTTTCTGAAGTGCGAGGCCCCAAACTACAGCGG |
| | | ACGGTTCACCTGTTCCTGGCTGGTGCAGCGCAACATGG |
| | | ACCTGAAGTTTAACATCAAGAGCAGCAGCAGCAGCCCC |
| | | GATTCTCGGGCTGTGACATGTGGCATGGCCTCCCTGTCT |
| | | GCTGAGAAGGTGACCCTGGACCAGCGCGATTACGAGAA |
| | | GTACAGCGTGTCCTGCCAGGAGGACGTGACATGTCCAA |
| | | CCGCCGAGGAGACACTGCCAATCGAGCTGGCCCTGGAG |
| | | GCTAGGCAGCAGAACAAGTACGAGAACTACTCTACCAG |
| | | CTTCTTTATCCGCGACATCATCAAGCCTGATCCCCCTAA |
| | | GAACCTGCAGATGAAGCCACTGAAGAACTCCCAGGTCG |
| | | AGGTGTCTTGGGAGTACCCCGACTCCTGGTCTACACCTC |
| | | ACTCTTACTTCAGCCTGAAGTTCTTCGTGAGAATCCAGA |
| | | GAAAGAAGGAGAAGATGAAGGAGACCGAGGAGGGCTG |
| | | CAACCAGAAGGGAGCTTTTCTGGTGGAGAAGACAAGCA |
| | | CCGAGGTGCAGTGCAAGGGCGGAAACGTGTGCGTGCAG |
| | | GCCCAGGACAGGTACTACAACTCTAGCTGTTCCAAGTG |
| | | GGCTTGCGTGCCTTGTCGGGTGCGCTCTAGGAGAAAGA |
| | | GGGGCAGCGGAGAGGGAAGGGGCAGCCTGCTGACATG |
| | | CGGCGATGTGGAGGAGAACCCCGGACCTATGTGCCAGA |
| | | GCAGATACCTGCTGTTCCTGGCCACCCTGGCTCTGATCA |
| | | ACCACCTGTCTCTGGCCAGAGTGATCCCTGTGAGCGGC |
| | | CCAGCTAGGTGCCTGAGCCAGTCCAGAAACCTGCTGAA |
| | | GACCACAGACGATATGGTGAAGACAGCCAGGGAGAAG |
| | | CTGAAGCACTACTCCTGTACCGCTGAGGACATCGATCA |
| | | CGAGGACATCACAAGAGATCAGACATCCACCCTGAAGA |
| | | CCTGCCTGCCTCTGGAGCTGCACAAGAACGAGTCTTGTC |
| | | TGGCCACAAGGGAGACCTCCTCTACCACAAGAGGCTCC |
| | | TGCCTGCCACCCCAGAAGACATCTCTGATGATGACCCT |
| | | GTGCCTGGGAAGCATCTACGAGGACCTGAAGATGTACC |
| | | AGACAGAGTTCCAGGCCATCAACGCCGCTCTGCAGAAC |
| | | CACAACCACCAGCAGATCATCCTGGACAAGGGCATGCT |
| | | GGTGGCTATCGATGAGCTGATGCAGTCTCTGAACCACA |
| | | ACGGCGAGACCCTGAGGCAGAAGCCTCCAGTGGGAGA |
| | | GGCCGATCCATACAGAGTGAAGATGAAGCTGTGCATCC |
| | | TGCTGCACGCTTTTAGCACACGGGTGGTGACCATCAAC |
| | | CGCGTGATGGGCTACCTGAGCAGCGCCTGA |
| CAR2-P2A-HSV- | 42 | MALPVTALLLPLALLLHAARPDIQMTQTTSSLSASLGDRV |
| eTK-P2A-humanIL- | | TISCRASQDISKYLNWYQQKPDGTVKLLIYHTSRLHSGVPS |
| 12T | | RFSGSGSGTDYSLTISNLEQEDIATYFCQQGNTLPYTFGGG |
| | | TKLEITSSGGGGSGGGGGGSSRSSEVKLQESGPGLVAPSQS |
| | | LSVTCTVSGVSLPDYGVSWIRQPPRKGLEWLGVIWGSETT |
| | | YYNSALKSRLTIIKDNSKSQVFLKMNSLQTDDTAIYYCAK |
| | | HYYYGGSYAMDYWGQGTSVTVSSTTTPAPRPPTPAPTIAS |
| | | QPLSLRPEACRPAAGGAVHTRGLDFACDIYIWAPLAGTCG |
| | | VLLLSLVITLYCKRGRKKLLYIFKQPFMRPVQTTQEEDGCS |
| | | CRFPEEEEGGCGLRVKFSRSADAPAYQQGQNQLYNELNL |
| | | GRREEYDVLDKRRGRDPEMGGKPRRKNPQEGLYNELQK |
| | | DKMAEAYSEIGMKGERRRGKGHDGLYQGLSTATKDTYD |
| | | ALHMQALPPRGSGATNFSLLKQAGDVEENPGPMALQKKL |
| | | EELELDGSYPGHQHASAFDQAARSRGHSNGSTALRPGSQQ |
| | | EATEVRPEQKMPTLLRVYIDGPHGMGKTTTTQLLVALGSR |
| | | DDIVYVPEPMTYWRVLGASETIANIYTTQHRLDQGEISAG |
| | | DAAVVMTSAQITMGMPYAVTDAVLAPHIGGEAGSSHAPP |
| | | PALTLIFDRHPIAHLLCYPAARYLMGSMTPQAVLAFVALIP |
| | | PTLPGTNIVLGALPEDRHIDRLAKRQRPGERLDLAMLAAIR |
| | | RVYGLLANTVRYLQCGGSWREDWGQLSGTAVPPQGAEP |
| | | QSNAGPRPHIGDTLFTLFRAPELLAPNGDLYNVFAWALDV |
| | | LAKRLRSMHVFILDYDQSPAGCRDALLQLTSGMVQTHVT |
| | | TPGSIPTICDLARTFAREMGEANGSGATNFSLLKQAGDVEE |
| | | NPGPMCHQQLVISWFSLVFLASPLVAIWELKKDVYVVELD |
| | | WYPDAPGEMVVLTCDTPEEDGITWTLDQSSEVLGSGKTL |
| | | TIQVKEFGDAGQYTCHKGGEVLSHSLLLLHKKEDGIWSTD |
| | | ILKDQKEPKNKTFLRCEAKNYSGRFTCWWLTTISTDLTFS |
| | | VKSSRGSSDPQGVTCGAATLSAERVRGDNKEYEYSVECQ |
| | | EDSACPAAEESLPIEVMVDAVHKLKYENYTSSFFIRDIIKPD |
| | | PPKNLQLKPLKNSRQVEVSWEYPDTWSTPHSYFSLTFCVQ |
| | | VQGKSKREKKDRVFTDKTSATVICRKNASISVRAQDRYYS |
| | | SSWSEWASVPCSRRKRGSGEGRGSLLTCGDVEENPGPMC |
| | | PARSLLLVATLVLLDHLSLARNLPVATPDPGMFPCLHHSQ |
| | | NLLRAVSNMLQKARQTLEFYPCTSEEIDHEDITKDKTSTVE |
| | | ACLPLELTKNESCLNSRETSFITNGSCLASRKTSFMMALCL |
| | | SSIYEDLKMYQVEFKTMNAKLLMDPKRQIFLDQNMLAVI |
| | | DELMQALNFNSETVPQKSSLEEPDFYKTKIKLCILLHAFRI |
| | | RAVTIDRVMSYLNAS |
| CAR2-P2A-HSV- | 43 | MALPVTALLLPLALLLHAARPDIQMTQTTSSLSASLGDRV |
| eTK-P2A-humanIL- | | TISCRASQDISKYLNWYQQKPDGTVKLLIYHTSRLHSGVPS |
| 12G | | RFSGSGSGTDYSLTISNLEQEDIATYFCQQGNTLPYTFGGG |
| | | TKLEITSSGGGGSGGGGGGSSRSSEVKLQESGPGLVAPSQS |
| | | LSVTCTVSGVSLPDYGVSWIRQPPRKGLEWLGVIWGSETT |
| | | YYNSALKSRLTIIKDNSKSQVFLKMNSLQTDDTAIYYCAK |
| | | HYYYGGSYAMDYWGQGTSVTVSSTTTPAPRPPTPAPTIAS |
| | | QPLSLRPEACRPAAGGAVHTRGLDFACDIYIWAPLAGTCG |
| | | VLLLSLVITLYCKRGRKKLLYIFKQPFMRPVQTTQEEDGCS |
| | | CRFPEEEEGGCGLRVKFSRSADAPAYQQGQNQLYNELNL |
| | | GRREEYDVLDKRRGRDPEMGGKPRRKNPQEGLYNELQK |
| | | DKMAEAYSEIGMKGERRRGKGHDGLYQGLSTATKDTYD |
| | | ALHMQALPPRGSGATNFSLLKQAGDVEENPGPMALQKKL |
| | | EELELDGSYPGHQHASAFDQAARSRGHSNGSTALRPGSQQ |
| | | EATEVRPEQKMPTLLRVYIDGPHGMGKTTTTQLLVALGSR |
| | | DDIVYVPEPMTYWRVLGASETIANIYTTQHRLDQGEISAG |
| | | DAAVVMTSAQITMGMPYAVTDAVLAPHIGGEAGSSHAPP |
| | | PALTLIFDRHPIAHLLCYPAARYLMGSMTPQAVLAFVALIP |
| | | PTLPGTNIVLGALPEDRHIDRLAKRQRPGERLDLAMLAAIR |
| | | RVYGLLANTVRYLQCGGSWREDWGQLSGTAVPPQGAEP |
| | | QSNAGPRPHIGDTLFTLFRAPELLAPNGDLYNVFAWALDV |
| | | LAKRLRSMHVFILDYDQSPAGCRDALLQLTSGMVQTHVT |
| | | TPGSIPTICDLARTFAREMGEANGSGATNFSLLKQAGDVEE |
| | | NPGPMCHQQLVISWFSLVFLASPLVAIWELKKDVYVVELD |
| | | WYPDAPGEMVVLTCDTPEEDGITWTLDQSSEVLGSGKTL |
| | | TIQVKEFGDAGQYTCHKGGEVLSHSLLLLHKKEDGIWSTD |
| | | ILKDQKEPKNKTFLRCEAKNYSGRFTCWWLTTISTDLTFS |
| | | VKSSRGSSDPQGVTCGAATLSAERVRGDNKEYEYSVECQ |
| | | EDSACPAAEESLPIEVMVDAVHKLKYENYTSSFFIRDIIKPD |
| | | PPKNLQLKPLKNSRQVEVSWEYPDTWSTPHSYFSLTFCVQ |
| | | VQGKSKREKKDRVFTDKTSATVICRKNASISVRAQDRYYS |
| | | SSWSEWASVPCSGGGGSGGGGSGGGGSMCPARSLLLVAT |
| | | LVLLDHLSLARNLPVATPDPGMFPCLHHSQNLLRAVSNM |
| | | LQKARQTLEFYPCTSEEIDHEDITKDKTSTVEACLPLELTK |
| | | NESCLNSRETSFITNGSCLASRKTSFMMALCLSSIYEDLKM |
| | | YQVEFKTMNAKLLMDPKRQIFLDQNMLAVIDELMQALNF |
| | | NSETVPQKSSLEEPDFYKTKIKLCILLHAFRIRAVTIDRVMS |
| | | YLNAS |
| CAR2-SV40-HSV- | 44 | ATGGCCTTACCAGTGACCGCCTTGCTCCTGCCGCTGGCC |
| eTK | | TTGCTGCTCCACGCCGCCAGGCCGGACATCCAGATGAC |
| | | ACAGACTACATCCTCCCTGTCTGCCTCTCTGGGAGACAG |
| | | AGTCACCATCAGTTGCAGGGCAAGTCAGGACATTAGTA |
| | | AATATTTAAATTGGTATCAGCAGAAACCAGATGGAACT |
| | | GTTAAACTCCTGATCTACCATACATCAAGATTACACTCA |
| | | GGAGTCCCATCAAGGTTCAGTGGCAGTGGGTCTGGAAC |
| | | AGATTATTCTCTCACCATTAGCAACCTGGAGCAAGAAG |
| | | ATATTGCCACTTACTTTTGCCAACAGGGTAATACGCTTC |
| | | CGTACACGTTCGGAGGGGGGACCAAGCTGGAGATCACA |
| | | TCCTCTGGCGGCGGCGGCTCTGGCGGCGGAGGAGGAGG |
| | | CAGCTCCAGGTCTAGCGAGGTGAAACTGCAGGAGTCAG |
| | | GACCTGGCCTGGTGGCGCCCTCACAGCGCCTGTCCGTC |
| | | ACATGCACTGTCTCAGGGGTCTCATTACCCGACTATGGT |
| | | GTAAGCTGGATTCGCCAGCCTCCACGAAAGGGTCTGGA |
| | | GTGGCTGGGAGTAATATGGGGTAGTGAAACCACATACT |
| | | ATAATTCAGCTCTCAAATCCAGACTGACCATCATCAAG |
| | | GACAACTCCAAGAGCCAAGTTTTCTTAAAAATGAACAG |
| | | TCTGCAAACTGATGACACAGCCATTTACTACTGTGCCAA |
| | | ACATTATTACTACGGTGGTAGCTATGCTATGGACTACTG |
| | | GGGCCAAGGAACCTCAGTCACCGTCTCCTCAACCACGA |
| | | CGCCAGCGCCGCGACCACCAACACCGGCGCCCACCATC |
| | | GCGTCGCAGCCCCTGTCCCTGCGCCCAGAGGCGTGCCG |
| | | GCCAGCGGCGGGGGGCGCAGTGCACACGAGGGGGCTG |
| | | GACTTCGCCTGTGATATCTACATCTGGGCGCCCTTGGCC |
| | | GGGACTTGTGGGGTCCTTCTCCTGTCACTGGTTATCACC |
| | | CTTTACTGCAAACGGGGCAGAAAGAAACTCCTGTATAT |
| | | ATTCAAACAACCATTTATGAGACCAGTACAAACTACTC |
| | | AAGAGGAAGATGGCTGTAGCTGCCGATTTCCAGAAGAA |
| | | GAAGAAGGAGGATGTGGACTGAGAGTGAAGTTCAGCA |
| | | GGAGCGCAGACGCCCCCGCGTACCAGCAGGGCCAGAA |
| | | CCAGCTCTATAACGAGCTCAATCTAGGACGAAGAGAGG |
| | | AGTACGATGTTTTGGACAAGAGACGTGGCCGGGACCCT |
| | | GAGATGGGGGGAAAGCCGAGAAGGAAGAACCCTCAGG |
| | | AAGGCCTGTACAATGAACTGCAGAAAGATAAGATGGCG |
| | | GAGGCCTACAGTGAGATTGGGATGAAAGGCGAGCGCC |
| | | GGAGGGGCAAGGGGCACGATGGCCTTTACCAGGGTCTC |
| | | AGTACAGCCACCAAGGACACCTACGACGCCCTTCACAT |
| | | GCAGGCCCTGCCCCCTCGCTAATAGGGATCCATCGGAT |
| | | CCCGGGCCCGTCGACTCGCTGTGGAATGTGTGTCAGTTA |
| | | GGGTGTGGAAAGTCCCCAGGCTCCCCAGCAGGCAGAAG |
| | | TATGCAAAGCATGCATCTCAATTAGTCAGCAACCAGGT |
| | | GTGGAAAGTCCCCAGGCTCCCCAGCAGGCAGAAGTATG |
| | | CAAAGCATGCATCTCAATTAGTCAGCAACCATAGTCCC |
| | | GCCCCTAACTCCGCCCATCCCGCCCCTAACTCCGCCCAG |
| | | TTCCGCCCATTCTCCGCCCCATGGCTGACTAATTTTTTTT |
| | | ATTTATGCAGAGGCCGAGGCCGCCTCGGCCTCTGAGCT |
| | | ATTCCAGAAGTAGTGAGGAGGCTTTTTTGGAGGCCTAG |
| | | GCTTTTGCAAAAAGCTCGAAGATCAATTCCGATCTGATC |
| | | AACCGGTACGCGTCCACCATGGCCCTGCAGAAAAAGCT |
| | | GGAAGAGCTGGAACTGGATGGCAGCTACCCCGGCCACC |
| | | AGCACGCCAGCGCCTTCGACCAGGCCGCCCGCAGCCGC |
| | | GGCCACAGCAACGGCAGCACCGCACTGCGGCCAGGATC |
| | | TCAGCAGGAGGCCACCGAGGTGCGCCCCGAGCAGAAG |
| | | ATGCCCACCCTGCTGCGCGTGTACATCGACGGACCACA |
| | | CGGCATGGGCAAGACCACCACCACCCAGCTGCTGGTGG |
| | | CCCTGGGCAGCCGCGACGACATCGTGTACGTGCCCGAG |
| | | CCCATGACCTACTGGCGCGTGCTGGGCGCCAGCGAGAC |
| | | CATCGCCAACATCTACACCACCCAGCACCGCCTGGACC |
| | | AAGGCGAGATCAGCGCCGGCGACGCCGCCGTGGTGATG |
| | | ACCAGCGCCCAGATTACAATGGGCATGCCCTACGCCGT |
| | | GACCGACGCCGTGCTGGCACCACACATCGGCGGCGAGG |
| | | CCGGCAGCAGCCACGCACCACCACCAGCACTGACCCTG |
| | | ATCTTCGACCGGCACCCAATCGCACACCTGCTGTGCTAC |
| | | CCGGCAGCACGCTACCTGATGGGCTCCATGACACCACA |
| | | AGCCGTGCTGGCCTTCGTGGCCCTGATCCCACCAACACT |
| | | GCCCGGCACCAACATCGTGCTGGGCGCCCTGCCCGAGG |
| | | ACCGCCACATCGACCGCCTGGCCAAGCGCCAGCGCCCC |
| | | GGCGAGCGCCTGGACCTGGCCATGCTGGCCGCCATCCG |
| | | CCGCGTGTACGGCCTGCTGGCCAACACCGTGCGCTACC |
| | | TGCAGTGCGGCGGCAGCTGGCGCGAGGACTGGGGCCAG |
| | | CTGAGCGGCACCGCCGTGCCACCACAGGGCGCCGAGCC |
| | | ACAGAGCAACGCCGGACCACGACCACACATCGGCGAC |
| | | ACCCTGTTCACCCTGTTCCGGGCACCAGAGCTGCTGGCA |
| | | CCAAACGGCGACCTGTACAACGTGTTCGCCTGGGCCCT |
| | | GGACGTGCTGGCCAAGCGCCTGCGCTCCATGCACGTGT |
| | | TCATCCTGGACTACGACCAGTCACCGGCCGGCTGCCGC |
| | | GACGCCCTGCTGCAGCTGACCAGCGGCATGGTGCAGAC |
| | | CCACGTGACAACACCCGGCAGCATCCCAACAATCTGCG |
| | | ACCTGGCCCGCACCTTCGCCCGCGAGATGGGCGAGGCC |
| | | AACTAA |
| Modified Sindbis | 45 | MASAAPLVTAMCLLGNVSFPCDRPPTCYTREPSRALDILE |
| virus envelope | | ENVNHEAYDTLLNAILRCGSSGRSKRSVIDDFTLTSPYLGT |
| encoding CD8 scFv | | CSYCHHTVPCFSPVKIEQVWDEADDNTIRIQTSAQFGYDQ |
| targeting motif | | SGAASANKYRYMAAAAVTAAGHVGEVQLVESGGGLVQP |
| | | GRSLRLSCAASGFTFDDYTMHWVRQAPGKGLEWVSGISW |
| | | NSGSIGYADSVKGRFTISRDNAKKSLYLQMNSLRAEDTAL |
| | | YYCAKDNSGYGHYYYGMDVWGQGTTVTVASASTKGGG |
| | | GGSGGGGSGGGGSEIVMTQSPATLSVSPGERATLSCRASQ |
| | | SVSSNLAWYQQKPGQAPRLLIYGASTRATGIPARFSGSGS |
| | | GTEFTLTISSLQSEDFAVYYCQHYINWPLTFGGGTKVEIKG |
| | | VHGAAVTTVKEGTMDDIKISTSGPCRRLSYKGYFLLAKCP |
| | | PGDSVTVSIVSSNSATSCTLARKIKPKFVGREKYDLPPVHG |
| | | KKIPCTVYDRLAATTAGYITMHRPRPHAYTSYLEESSGKV |
| | | YAKPPSGKNITYECKCGDYKTGTVSTRTEITGCTAIKQCV |
| | | AYKSDQTKWVFNSPDLIRHDDHTAQGKLHLPFKLIPSTCM |
| | | VPVAHAPNVIHGFKHISLQLDTDHLTLLTTRRLGANPEPTT |
| | | EWIVGKTVRNFTVDRDGLEYIWGNHEPVRVYAQESAPGD |
| | | PHGWPHEIVQHYYHRHPVYTILAVASATVAMMIGVTVAV |
| | | LCACKARRECLTPYALAPNAVIPTSLALLCCVRSANAETFT |
| | | ETMSYLWSNSQPFFWVQLCIPLAAFIVLMRCCSCCLPFLV |
| | | VAGAYLAKVDAYEHATTVPNVPQIPYKALVERAGYAPLN |
| | | LEITVMSSEVLPSTNQEYITCKFTTVVPSPKIKCCGSLECQP |
| | | AAHADYTCKVFGGVYPFMWGGAQCFCDSENSQMSEAYV |
| | | ELSADCASDHAQAIKVHTAAMKVGLRIVYGNTTSFLDVY |
| | | VNGVTPGTSKDLKVIAGPISASFTPFDHKVVIHRGLVYNY |
| | | DFPEYGAMKPGAFGDIQATSLTSKDLIASTDIRLLKPSYGN |
| | | VHVPYTQASSGFEMWKNNSGRPLQETAPFGCKIAVNPLR |
| | | AVDCSYGNIPISIDIPNAAFIRTSDAPLVSTVKCEVSECTYS |
| | | ADFGGMATLQYVSDREGQCPVHSHSSTATLQESTVHVLE |
| | | KGAVTVHFSTASPQANFIVSLCGKKTTCNAECKPPADHIV |
| | | STPHKNDQEFQAAISKTSWSWLFALFGGASSLLIIGLMIFA |
| | | CSMMLTSTRR |

### EXAMPLES

### Example 1: Viral Vectors with a Modified Sindbis Virus Envelope Specifically Transduce CD8⁺ Cell

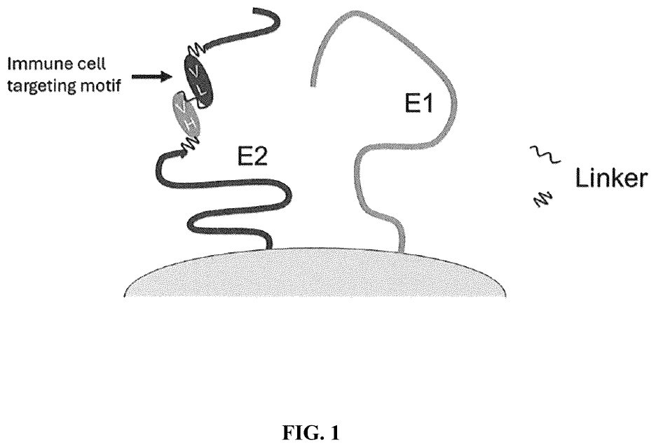

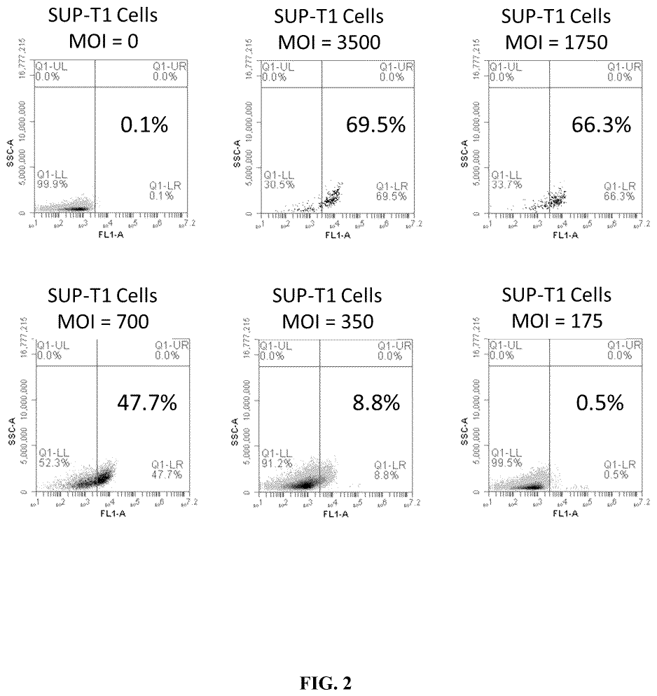

Vectors with a modified Sindbis virus envelope (SEQ ID NO: 1) have been generated bearing a CD8 targeting motif (scFv) expressed within the Sindbis virus envelope. A schematic structure of the vectors is shown in FIG. 1. Human cell lines, Jurkat (CD8⁻) and SUP-T1 (CD8⁺), were transduced with these CD8 targeted vectors encoding a copGFP reporter payload. copGFP expression was examined 5 days post-transduction. FIG. 2 and FIG. 3 show that SUP-T1 cells expressed the copGFP reporter protein at a significantly higher rate than Jurkat cells, with infectivity increasing as the multiplicity of infection (MOI) increased. The result suggests that the viral vector with a modified Sindbis virus envelope specifically transduce CD8+ cell.

### Example 2: In Vitro Transduction Efficiency and Cytotoxicity of Engineered Viral Vectors Encoding a Chimeric Antigen Receptor (CAR)

### In Vitro Transduction Efficiency

### Materials and Methods

PBMC Transduction: Twelve-well cell culture plates were coated with a recombinant fragment of human fibronectin (RectroNectin®; Takara) per manufacturer's instructions and subsequently with CAR-encoding vectors produced by standard transient transfection methods in HEK293T cells in a range of 0.5-2.0×10⁹ vector genomes per milliliter. Vectors comprised a non-replicating gammaretrovector derived from mouse leukemia virus (MLV). To each such well was added 2.0×10⁵ human PBMCs that had been thawed three days prior using the cell culture media of X-Vivo-15 (Lonza catalog 02-053Q) with 10% Human Male AB heat-inactivated serum (BIOIVT catalog number HUMANABSRMP-HI-1), with resting of PBMCs followed by stimulation with CD3/CD28 (DynaBeads; Gibco catalog number 11161 or Immunocult; STEMCELL catalog number 10971) per manufacturer's instructions, then 200 IU/mL IL-2 addition one day before transduction. The resulting PBMC suspensions were “spinoculated” by centrifugation for 45 minutes at 700×g to effect transduction of the stimulated PBMC cells by the vectors installed within each respective well. CellTracer Red was used at the time of thaw to verify cell division and stimulation during early transduction/CAR expression experiments; in general, PBMC propagation in both stimulated and unstimulated samples was verified by propidium iodide staining of separate dedicated samples near the time of transduction. One day after transduction, cells were removed from the twelve-well cell culture and spun down at 500×g for 5 mins, then cultured in 3 ml of 200 IU IL12/mL in a six-well culture plate.

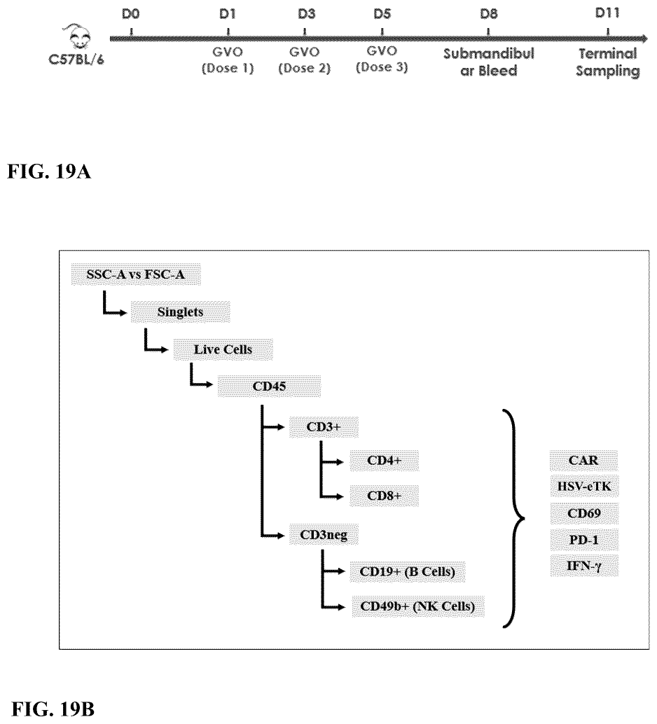

PBMC FACS analyses: Three days after transduction, PBMC cells were Fc blocked, stained with surface markers, fixed using 4% paraformaldehyde solution, and labeled with fluorescent antibodies using standard methodologies and reagents per the panels listed herein (Table 3). Analyses of intracellular proteins (e.g., HSV-eTK) were accommodated using appropriate and readily available permeabilization reagents (e.g. 10× permeabilization buffer from Invitrogen, catalog number 00-8333-56, or similar) in separate dedicated samples. Analyses were conducted in parallel in 96-well format to minimize differential delays in sample preparation and processing. Flow data were gated for singlets (at side-scatter vs. forward-scatter) and for lack of uptake of free amines using Fixable Aqua dye (so-called “live-dead” staining) (FIG. 19B). Subsequent analyses focused on CD3(+) cells (including CD4(+) and CD8(+) subpopulations) and CD3(−) cells (including CD19(+) and CD56(+) subpopulations for B- and NK-cells, respectively). All populations above were then assessed for the percentage of cells fluorescent for the anti-CAR labeled tag within the panel (i.e., PE-labeled CD19-his tagged protein).

TABLE 3
FACS reagents

| | | | | Target | |
|---|---|---|---|---|---|
| Reagent | Manufacturer | Cat # | Dilution | Species | Host |
| PE His Tag CD19 | Acro Biosystems | CD9-HP2H3 | 1:50 | Human | N/A |
| APC-Cy7 anti-human | ThermoFisher | A15440 | 1:100 | Human | Mouse |
| CD3e | | | | | |
| PE-Texas Red anti-human | ThermoFisher | MHCD0417 | 1:100 | Human | Mouse |
| CD4 | | | | | |
| Pacific Blue anti-human | ThermoFisher | MHCD0828 | 1:100 | Human | Mouse |
| CD8 | | | | | |
| PE-Cy7 anti-human CD56 | BD Biosciences | BDB560916 | 1:100 | Human | Mouse |
| SuperBright 702 anti- | ThermoFisher | 67-0199-42 | 1:100 | Human | Mouse |
| human CD19 | | | | | |
| L/D Fixable Aqua | ThermoFisher | L34957 | 1:1000 | N/A | N/A |
| v-eTK Alexa-Fluor-647 | Genscript | GenVivo | 1:200 | N/A | Goat |
| (intracellular) | | | | | |

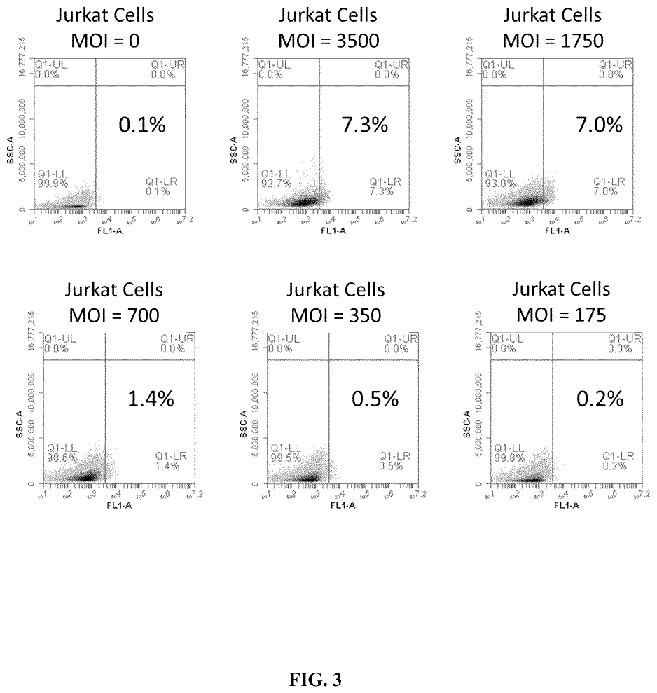

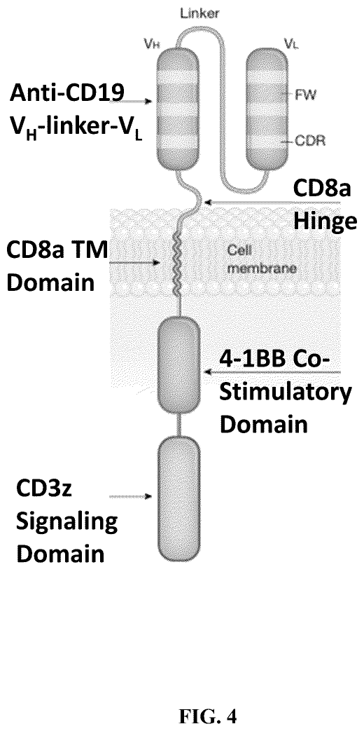

Human peripheral blood mononuclear cells PBMCs were transduced with non-replicating gammaretrovectors derived from mouse leukemia virus (MLV) with chimeric antigen receptor (CAR)-containing payloads to evaluate their viability in the context of an HSV-TK gene and relative activity with respect to three V_{H}-V_{L} linker options. The CARs bind the B-cell receptor CD19 via the V_{H} and V_{L} domains. The three V_{H}-V_{L} linker options were tested: GSTSGSGKPGSGEGSTKG, the Whitlow linker, denoted as CAR0 (SEQ ID NO: 18); GGGGSGGGGSGGGGS, denoted as CAR1 (SEQ ID NO: 19); or SSGGGGSGGGGGGSSRSS, denoted as CAR2 (SEQ ID NO: 11). The retrovectors additionally comprise a 4070A envelope and a gagpol gene cassette. A diagram depicting the domains of an example CAR is provided in FIG. 4.

As used herein, “HSV-eTK,” “eTK,” “vTK,” or “v-eTK” are all synonymous with “TK”.

To assess the transduction efficiency and activity of the CAR constructs, PBMCs collected from two human donors (Donors 1 and 2) were first thawed on day 0. On day 1, the PBMCs were stimulated with a CD3/CD28 antibody cocktail. The antibody cocktail was administered either in the form of removable beads coated with the antibodies, or as an antibody mixture in solution. On day 2, 200 IU/mL of IL2 was added. (In cases where CD3/CD28 was administered on beads, the beads were removed prior to IL2 addition.) On day 3, 43 hours post-CD3/CD28 stimulation and 24 hours post-IL2 addition, the PBMCs were transduced with vectors expressing the CAR constructs. Concomitantly, 20 μg/mL Retronectin was added at a MOI of 350, and the cells were subjected to spinoculation at 0.7×g for 45 minutes. On day 6, 3 days post-transduction, FACS was performed to interrogate transduction efficiency.

### Results

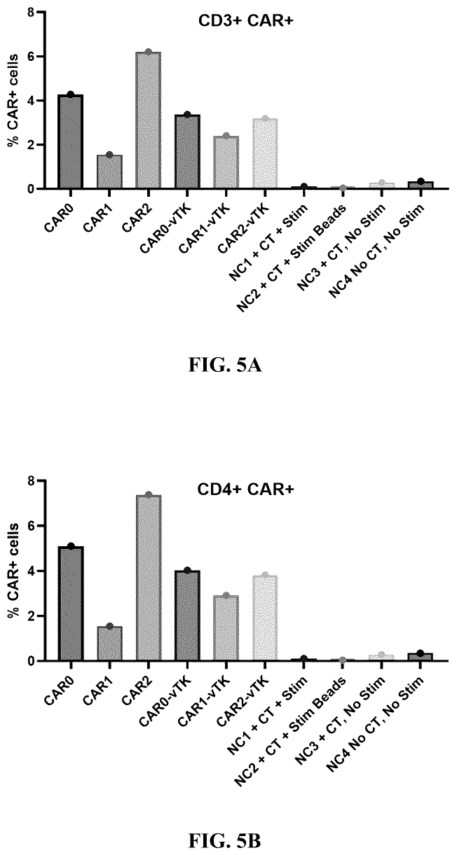

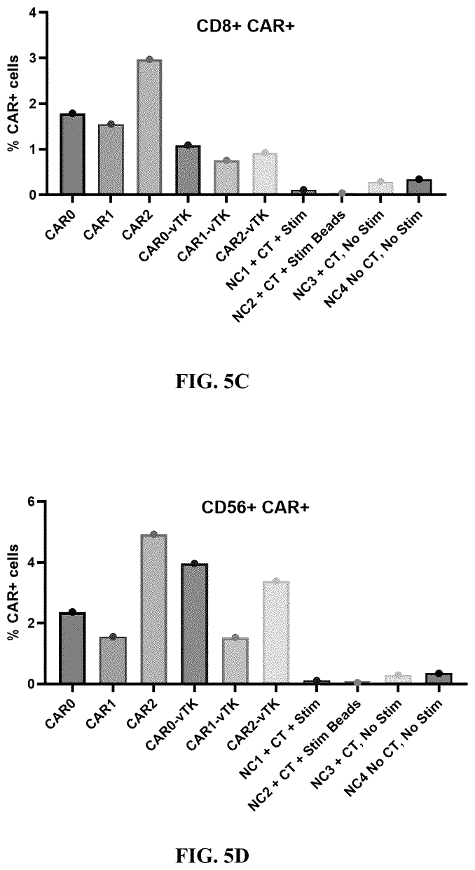

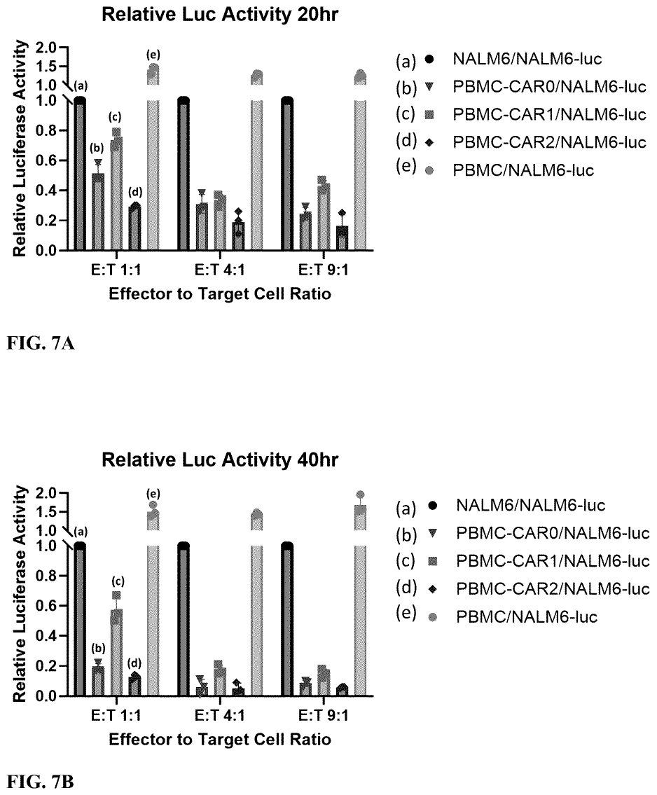

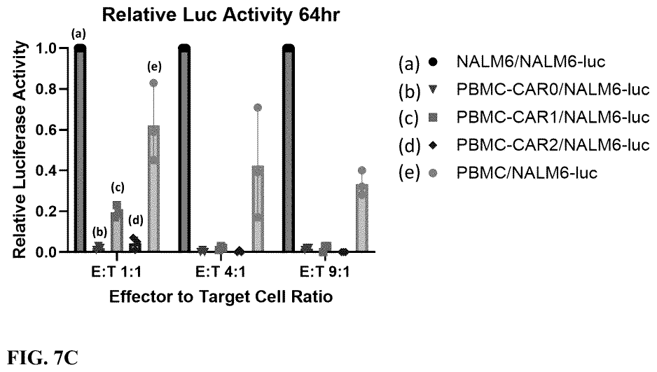

FIGS. 5A-5D show the percentage of CAR-expressing human peripheral blood mononuclear cells (PBMCs) following transduction with vectors expressing CAR0 (GSTSGSGKPGSGEGSTKG; Whitlow linker; SEQ ID NO: 18), CAR1 (GGGGSGGGGSGGGGS; SEQ ID NO: 19), or CAR2 (SSGGGGSGGGGGGSSRSS; SEQ ID NO: 11), with or without HSV-TK (TK). As a negative control (NC), some vectors did not express CAR. Results are shown in CD3+ CAR+ PBMCs (FIG. 5A), CD4+ CAR+ PBMCs (FIG. 5B), CD8+ CAR+ PBMCs (FIG. 5C), and CD56+ CAR+ PBMCs (FIG. 5D). “Stim” denotes stimulation with an antibody mixture of CD3/CD28, and “Stim Beads” denotes stimulation with removable beads coated with CD3/CD28 antibodies. Of the three linkers tested, the CAR2 linker was found to have the best combination of expression (see, e.g., FIGS. 5A-5D) and CD19+ target cell cytotoxicity (see, e.g., FIGS. 7A-7C).

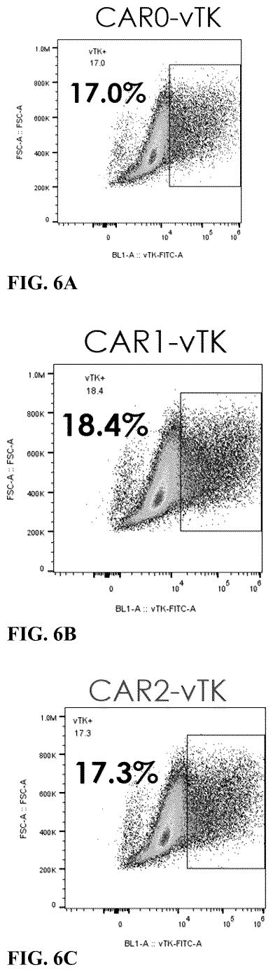

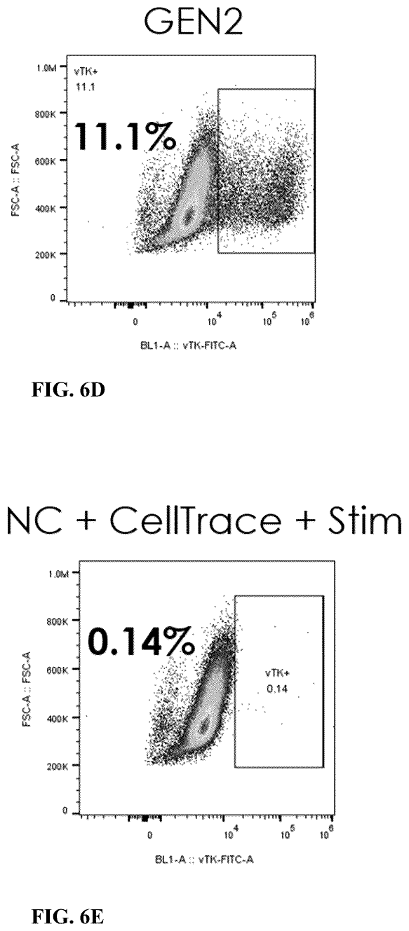

FIGS. 6A-6H show results from a FACS experiment demonstrating example TK expression levels across different CAR-TK constructs. FIG. 6A shows TK expression in PBMCs transduction with CAR0-TK. FIG. 6B shows TK expression in PBMCs transduction with CAR1-TK. FIG. 6C shows TK expression in PBMCs transduction with CAR2-TK. FIG. 6D shows TK expression in PBMCs transduction with a GEN2-type retrovector comprising a wild-type 4070A strain Moloney MLV envelope protein. FIG. 6E shows TK expression in PBMCs transduced with no construct (NC).

Overall, vectors expressing CAR2 exhibited the highest expression levels amongst the vectors tested. Further, addition of TK into the construct resulted in decreased CAR expression for CAR0 and CAR2 V_{H}-V_{L} linkers, but not for the CAR1 V_{H}-V_{L} linker. As expected, transduction with the negative control (GEN2), did not facilitate CAR expression (FIG. 6D).

### Cytotoxicity

Next, the potential for expression of the vectors provided herein to promote cytotoxicity against target B cell lines was tested.

### Materials and Methods

NALM6-luc and Raji-eGFP were used as target cell lines. Cells were plated at a target cell to effector cell ratio of 1:1, 1:4, or 1:9 (Table 2). Cells were plated in 96-well plates, and three technical replicates were performed. Further, 100 IU/mL IL2 was added to each sample. Luciferase or GFP activity was monitored over a 64-hour time period in the NALM6-luc or Raji-eGFP cell lines, respectively, to assess cytotoxicity.

Cytotoxicity assessment of transduced “effector” cells: 5×103 or 1×10⁴ target cells (NALM-6/Luciferase or Raji/eGFP) were co-incubated with transduced PBMC cells sampled three days post-transduction at a range of 0.02-9:1 total “effector”: target cell ratios using cell culture media of RPMI-1640 (Gibco catalog number A1049101), 1% penicillin/streptomycin (Gibco catalog number 15140122), and 10% heat-inactivated fetal calf serum (GeminiBio catalog number S11150 or equivalent). The transduced PBMC population, as estimated by CAR(+) FACS analysis, was ˜2-15% of this ratio. The reporter activity level (luciferase or eGFP-fluorescence) was assessed over 1-3 days post-incubation in a 96-well plate reader for luciferase activity and GFP flow cytometry; the best luciferase signal discrimination was generally found 1 day after co-incubation start using NALM-6/luciferase target cells, while optimal GFP signal discrimination was 2 days after Raji-eGFP target cell co-incubation.

TABLE 2
Number of target and effector cells per well based
on the indicated target to effector cell ratio.

| Ratio | Target Cells | Effector cells |
|---|---|---|
| 0:1 | 0 | 1.00E+04 |
| 1:1 | 5.00E+03 | 5.00E+03 |
| 1:4 | 2.00E+03 | 8.00E+03 |
| 1:9 | 1.00E+03 | 9.00E+03 |

### Results

FIGS. 7A-7C demonstrate the results of an example reporter gene assay in NAML6-luc B cells. The NAML6-luc B cells were cultured with one of several groups of “effector cells”: NALM6-luc B cells (NALM6), PBMCs transduced with a vector expressing CAR0 (PBMC-CAR0) (SEQ ID NO: 20), PBMCs transduced with a vector expressing CAR1 (PBMC-CAR1) (SEQ ID NO: 21), PBMCs transduced with a vector expressing CAR2 (PBMC-CAR2) (SEQ ID NO: 22), or untransduced PBMCs (PBMC). The assay can demonstrate the magnitude of cytotoxicity of PBMCs expressing each CAR construct against the target cell line (NAML6-luc B cells), wherein the level of relative luciferase activity is inversely proportional to cytotoxicity. 1:1, 4:1, or 9:1 ratios of effector to target cells (E:T) were tested. FIG. 7A shows relative luciferase activity after 20 hours. FIG. 7B shows relative luciferase activity after 40 hours. FIG. 7C shows relative luciferase activity after 64 hours.

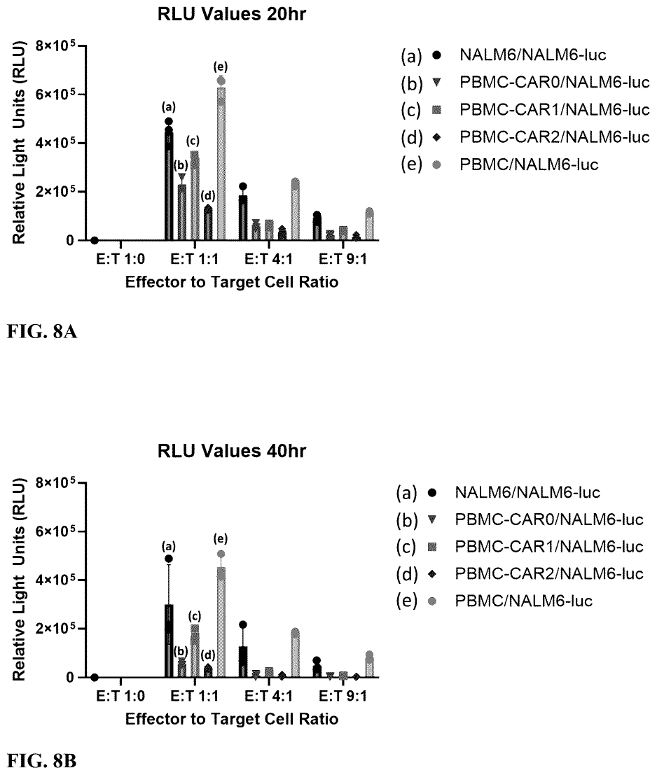

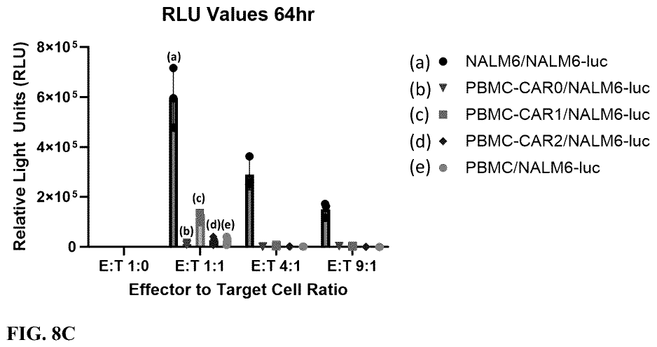

FIGS. 8A-8C demonstrate the results of an example reporter gene assay. NAML6-luc B cells were cultured with one of several groups of “effector cells”: NALM6-luc B cells (NALM6), PBMCs transduced with a vector expressing CAR0 (PBMC-CAR0) (SEQ ID NO: 20), PBMCs transduced with a vector expressing CAR1 (PBMC-CAR1) (SEQ ID NO: 21), PBMCs transduced with a vector expressing CAR2 (PBMC-CAR2) (SEQ ID NO: 22), or untransduced PBMCs (PBMC). The assay can demonstrate the magnitude of cytotoxicity of PBMCs expressing each CAR construct against the target cell line (NAML6-luc B cells), wherein the number of relative light units (RLUs) is inversely proportional to cytotoxicity. 1:1, 4:1, or 9:1 ratios of effector to target cells (E:T) were tested. FIG. 8A shows RLUs after 20 hours. FIG. 8B shows RLUs after 40 hours. FIG. 8C shows RLUs after 64 hours.

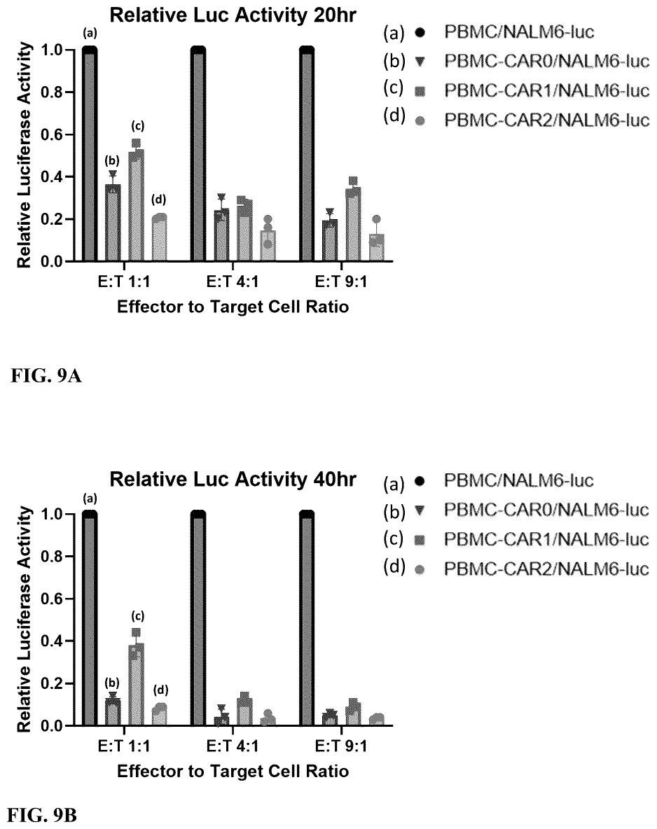

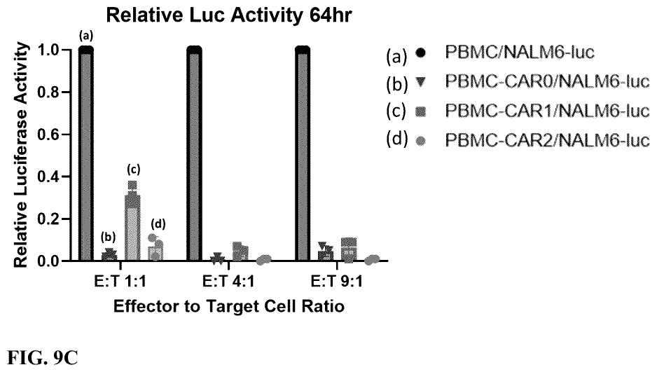

FIGS. 9A-9C demonstrate the results of an example reporter gene assay. NAML6-luc B cells were cultured with one of several groups of “effector cells”: PBMCs transduced with a vector expressing CAR0 (PBMC-CAR0) (SEQ ID NO: 20), PBMCs transduced with a vector expressing CAR1 (PBMC-CAR1) (SEQ ID NO: 21), PBMCs transduced with a vector expressing CAR2 (PBMC-CAR2) (SEQ ID NO: 22), or untransduced PBMCs (PBMC). The assay can demonstrate the magnitude of cytotoxicity of PBMCs expressing each CAR construct against the target cell line (NAML6-luc B cells), wherein the level of relative luciferase activity is inversely proportional to cytotoxicity. 1:1, 4:1, or 9:1 ratios of effector to target cells (E:T) were tested. Relative luciferase values were standardized such that the relative luciferase activity of NAML6-luc B cells cultured with untransduced PBMCs was set to 1.0. FIG. 9A shows relative luciferase activity after 20 hours. FIG. 9B shows relative luciferase activity after 40 hours. FIG. 9C shows relative luciferase activity after 64 hours.

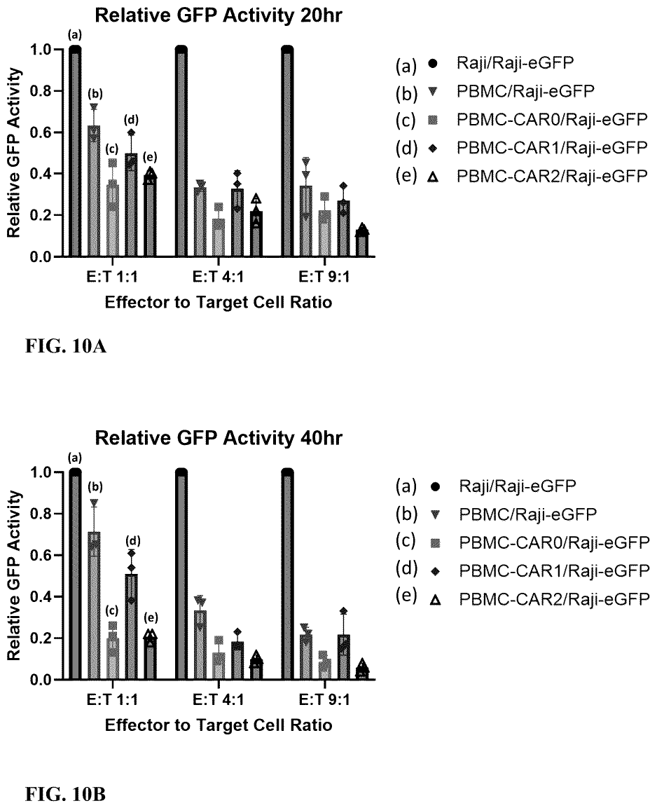

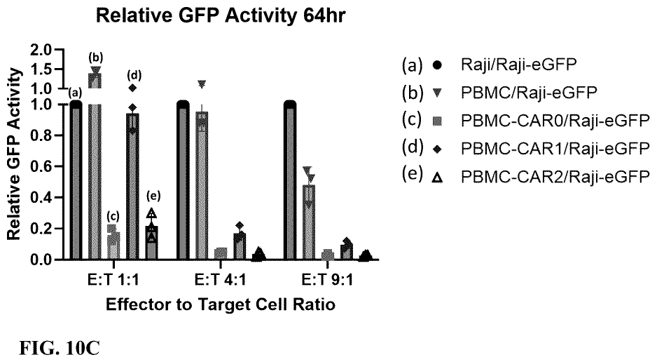

FIGS. 10A-10C demonstrate the results of an example reporter gene assay. Raji-eGFP cells were cultured with one of several groups of “effector cells”: Raji-eGFP cells (Raji), PBMCs transduced with a vector expressing CAR0 (PBMC-CAR0), PBMCs transduced with a vector expressing CAR1 (PBMC-CAR1), PBMCs transduced with a vector expressing CAR2 (PBMC-CAR2), or untransduced PBMCs (PBMC). The assay can demonstrate the magnitude of cytotoxicity of PBMCs expressing each CAR construct against the target cell line (Raji-eGFP cells), wherein the level of relative GFP activity is inversely proportional to cytotoxicity. 1:1, 4:1, or 9:1 ratios of effector to target cells (E:T) were tested. FIG. 10A shows relative GFP activity after 20 hours. FIG. 10B shows relative GFP activity after 40 hours. FIG. 10C shows relative GFP activity after 64 hours.

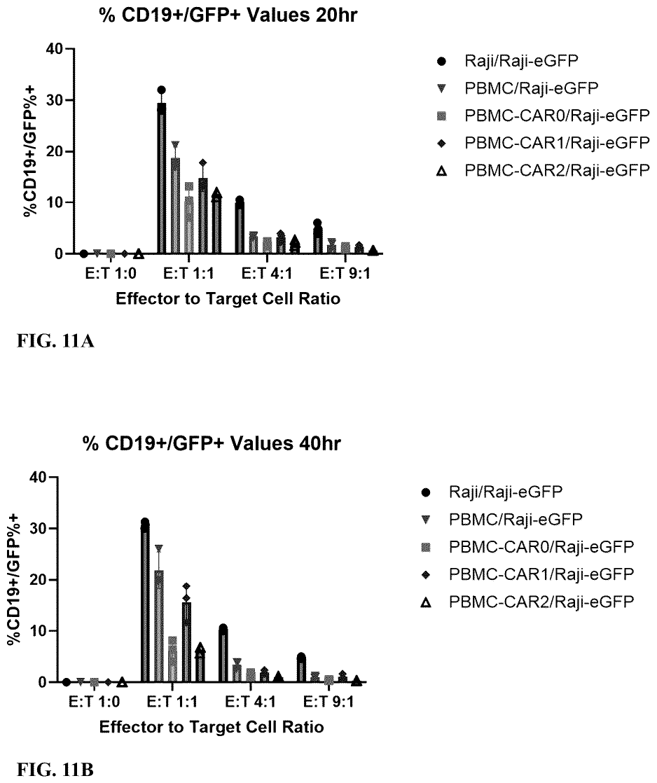

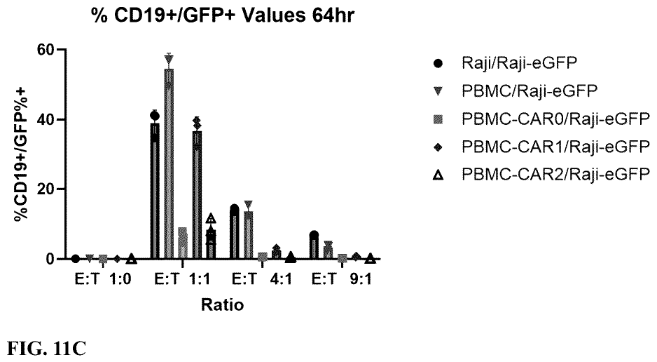

FIGS. 11A-11C demonstrate the results of an example reporter gene assay. Raji-eGFP cells were cultured with one of several groups of “effector cells”: Raji-eGFP cells (Raji), PBMCs transduced with a vector expressing CAR0 (PBMC-CAR0) (SEQ ID NO: 20), PBMCs transduced with a vector expressing CAR1 (PBMC-CAR1) (SEQ ID NO: 21), PBMCs transduced with a vector expressing CAR2 (PBMC-CAR2) (SEQ ID NO: 22), or untransduced PBMCs (PBMC). The assay can demonstrate the magnitude of cytotoxicity of PBMCs expressing each CAR construct against the target cell line (Raji-eGFP cells), wherein the percentage of cells expressing CD19 and GFP is inversely proportional to cytotoxicity. 1:0, 1:1, 4:1, or 9:1 ratios of effector to target cells (E:T) were tested. FIG. 11A shows the percentage of CD19 and GFP expression after 20 hours. FIG. 11B shows the percentage of CD19 and GFP expression after 40 hours. FIG. 11C shows the percentage of CD19 and GFP expression after 64 hours.

Overall, each CAR-expressing vector tested facilitated cytotoxicity against B cell lines NALM6-luc or Raji-eGFP, with CAR0- or CAR2-expressing vectors exhibiting the strongest cytotoxic effects.

The NALM-6/Luc system was elected for future studies as it showed higher sensitivity and dynamic range in the assays performed than did the Rai-GFP system. However, the Raji-GFP data were correlative with the NALM-6/Luc data.

### Example 3: Human T Cell Transduction Efficiency and Activity Against B-Lymphoma Cells of Engineered CAR-Encoding Viral Vectors

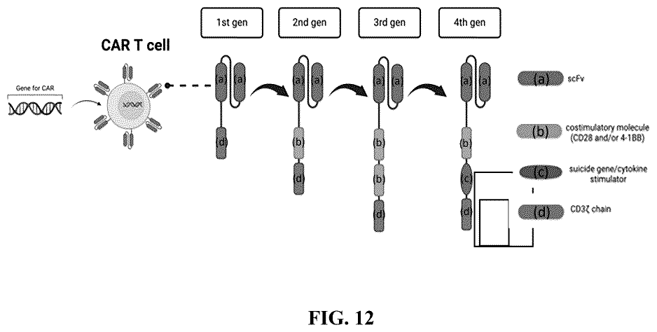

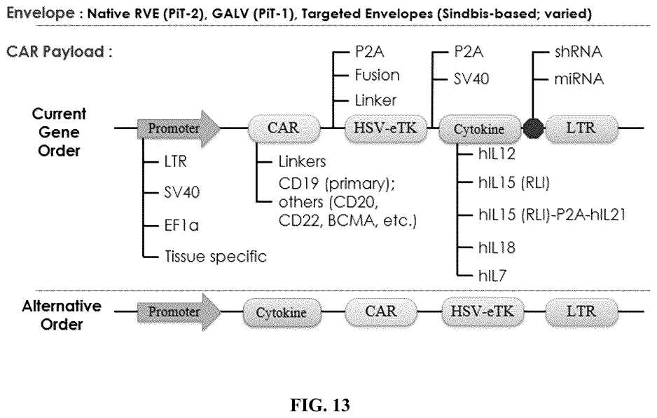

The transduction efficiency and cytotoxic activity of additional CAR-encoding vectors provided herein (see, e.g., Example 2) were evaluated. A diagram illustrating example 1st, 2nd, 3rd, and 4th generation CAR vectors is shown in FIG. 12. Vectors were designed which comprise, sequentially, a promoter region, a CAR region, an HSV-TK region, a cytokine region, and an LTR region. In an alternative order, the vector can comprise, sequentially, a promoter region, a cytokine region, a CAR region, an HSV-TK region, and an LTR region (FIG. 13).

### Materials and Methods

To assess the transduction efficiency and activity of the CAR constructs, PBMCs collected from two human donors (Donors 1 and 2) were first thawed on day 0. On day 1, the PBMCs were stimulated with a CD3/CD28 antibody cocktail. On day 2, 200 IU/mL of IL2 was added. (In cases where CD3/CD28 was administered on beads, the beads were removed prior to IL2 addition.) On day 3, 43 hours post-CD3/CD28 stimulation and 24 hours post-IL2 addition, the PBMCs were transduced with vectors expressing the CAR constructs. Concomitantly, 20 μg/mL Retronectin was added at a MOI of 350, and the cells were subjected to spinoculation at 0.7× g for 45 minutes. On day 6, 3 days post-transduction, FACS was performed to interrogate transduction efficiency. Additionally, transducted cells were seeded at 2×10⁵ cells per well on a 12-well plate for further analyses. On days 10 and 13, 6 or 10 days post-transduction, respectively, T cell activation panels and CAR PBMC panels were performed. Example vectors (i.e., plasmids) evaluated are shown below in Table 4.

Real-Time Cell Analysis (Cytotoxicity of effector cells): PBMC cells transduced with one of the CAR vectors co-encoding for IL-12 (Table 4) (SEQ ID NOS: 38-44) were added in 1:1 ratio to 96-well impedance plates of an xCELLigence real-time cell analysis (RTCA) instrument (Agilent) pre-loaded with 1×10⁴ target adherent A375 human melanoma cells engineered to express and display CD19 on the cell surface using cell culture media of RPMI-1640 (Gibco catalog number A1049101), 1% penicillin/streptomycin (Gibco catalog number 15140122), and 10% heat-inactivated fetal calf serum (GeminiBio catalog number S11150 or equivalent). The PBMCs were sampled at days 3, 6, and 10 post-transduction to match the “1:1” total effector:target ratios in triplicate and timepoints of the NALM-6 luciferase cytotoxicity assay (see Example 2). RTCA was allowed to proceed for five days (120 hours) by following the cell index, which measured the relative change in impedance and cell status every 15 minutes; this change in impedance over time is then inverted and presented as a percent cytolysis (greater cytolysis results in lowered impedance). Untransduced PBMCs were added to separate negative control wells on the same plate and under the same media conditions. In the presented experiment, PBMCs that were transduced with CAR vectors lacking an IL-12 gene cassette were employed with a 1:2 dilution using untransduced PBMC cells to adjust for the 3-fold higher level of transduction by CAR(+) FACS signal relative to the IL-12 encoding vectors.

TABLE 4
Plasmids evaluated for transduction efficiency
and activity in human PBMCs.

| # | Plasmid | SEQ ID NO |
|---|---|---|
| 1 | pCL-CAR2-P2A-veTK | 38 (polypeptide); 39 |
| | | (representative nucleotide) |
| 2 | pCL-CAR2-P2A-veTK-P2A-hIL12T | 42 (polypeptide) |
| 3 | pCL-CAR2-P2A-veTK-P2A-hIL12G | 43 (polypeptide) |
| 4 | pCL-CAR2-P2A-veTK-SV40-hIL12T | 40 (representative nucleotide) |
| 5 | pCL-CAR2-sv40-HSV-eTK | 44 |

### Results

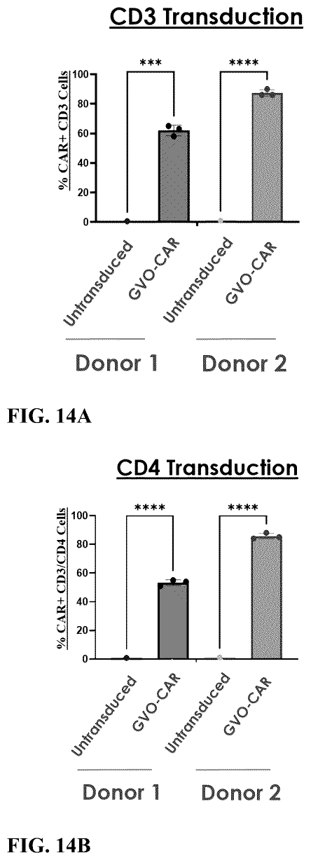

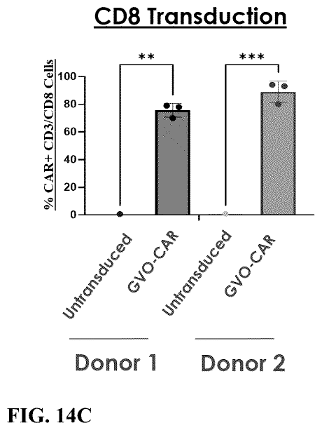

FIGS. 14A-14C show transduction efficiency, measured as the percentage of CAR-expressing CD3 cells (FIG. 14A), CD4 cells (FIG. 14B), or CD8 cells (FIG. 14C), in PBMCs from human Donors 1 and 2. GVO-CAR denotes a vector comprising LTR-CAR2-sv40-HSV-TK-LTR (SEQ ID NO: 44). GVO-CAR exhibited successful T cell transduction in human PBMCs.

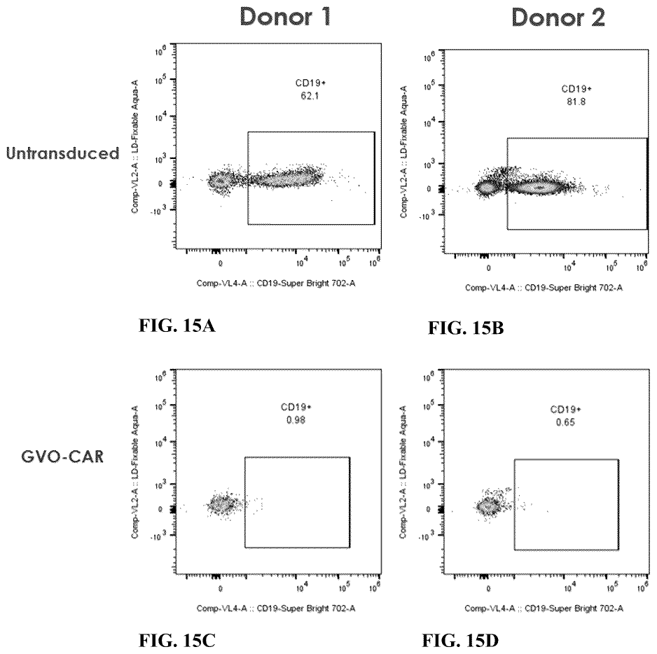

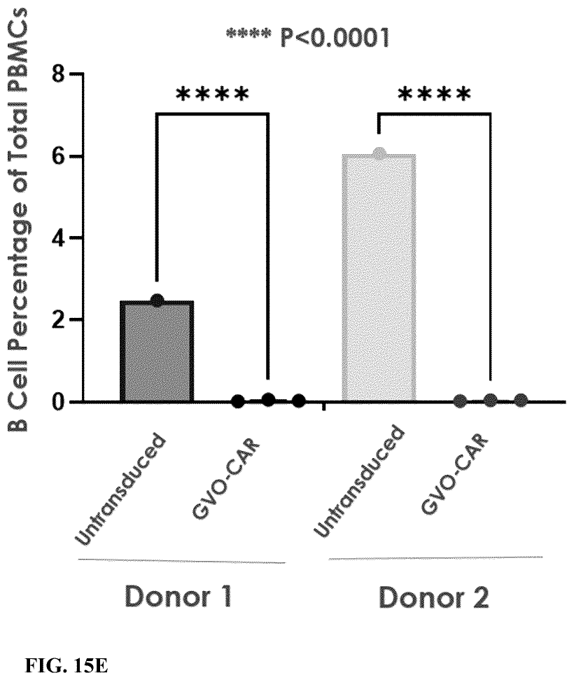

FIGS. 15A-15E show the magnitude of B cell depletion in human PBMC cultures with or without transduction with GVO-CAR (LTR-CAR2-sv40-HSV-TK-LTR) (SEQ ID NO: 44). FIG. 15A shows results of a flow cytometry the number of B cells in untransduced Donor 1 PBMCs. FIG. 15B shows results of a flow cytometry the number of B cells in untransduced Donor 2 PBMCs. FIG. 15C shows results of a flow cytometry the number of B cells in Donor 1 PBMCs transduced with GVO-CAR. FIG. 15D shows results of a flow cytometry the number of B cells in Donor 2 PBMCs transduced with GVO-CAR. FIG. 15E shows the percentage of B cells amongst the total pool of PBMCs in Donors 1 or 2 following either no transduction, or transduction with GVO-CAR. GVO-CAR exhibited successful B cell depletion in human PBMCs post-transduction.

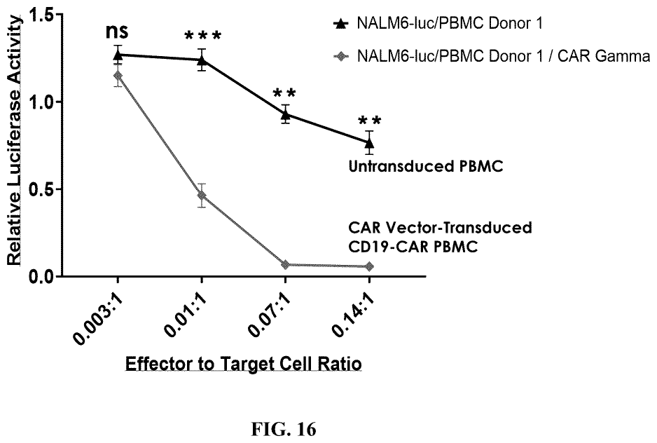

FIG. 16 shows relative luciferase activity as a function of the ratio of effector cells (PBMCs) to target cells (NALM6-luc). Triangles denote samples where the PBMCs were untransduced. Diamonds denote samples where the PBMCs were transduced with a vector expressing CD19-CAR. CAR vector-transduced PBMCs effectively killed B-lymphoma cells. Values provided in FIG. 16 account for both the transduction rate, as well as the effector to target ratios in each condition.

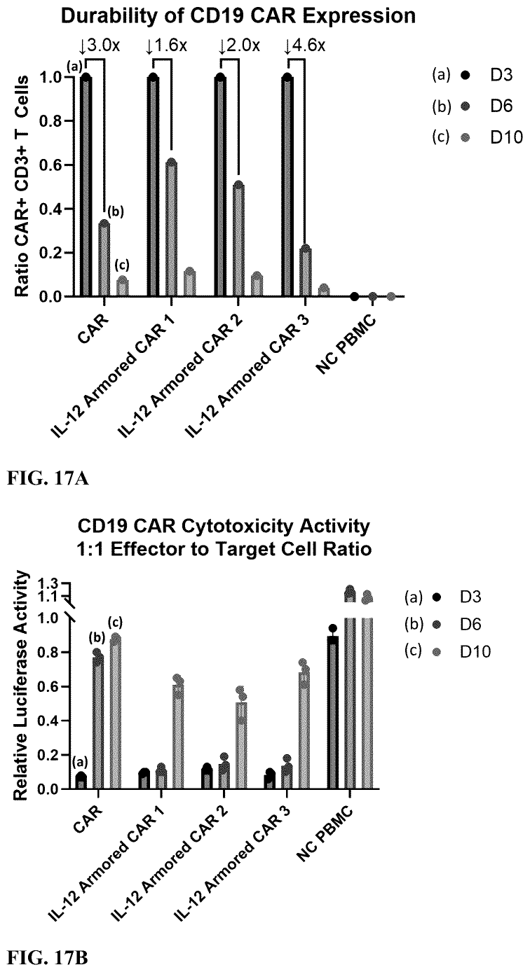

FIGS. 17A-17B illustrate example effects of further encoding IL-12 in a CAR-encoding vector on the durability of CAR vector expression and the magnitude of cytotoxic activity against target cells. FIG. 17A shows the magnitude of CAR vector expression in CD3+ T cells transduced with a vector comprising CAR2 p2a TK (CAR) (SEQ ID NO: 38), a vector comprising CAR2 p2a TK p2a IL12 (p40 t2a p35) (IL-12 Armored CAR 1) (SEQ ID NO: 42), a vector comprising CAR2 p2a TK p2a IL12 (p40 tg4sx3 p35) (IL-12 Armored CAR 2) (SEQ ID NO: 43), or a vector comprising CAR2 p2a TK SV40 IL12 (p40 t2a p35) (IL-12 Armored CAR 3) (SEQ ID NO: 40), or CD3+ T cells which were not transduced (NC PBMC). Results are shown 3 days (D3), 6 days (D6), or 10 days (D10) post-transduction. FIG. 17B shows the results of an example reporter gene assay. The assay can demonstrate the magnitude of cytotoxicity of the effector cells, PBMCs, expressing each CAR construct against the target cell line, wherein the level of relative luciferase activity is inversely proportional to cytotoxicity. The effector cells, PBMCs, were transduced with a vector comprising CAR2 p2a TK (CAR), a vector comprising CAR2 p2a TK p2a IL12 (p40 t2a p35) (IL-12 Armored CAR 1), a vector comprising CAR2 p2a TK p2a IL12 (p40 tg4sx3 p35) (IL-12 Armored CAR 2), or a vector comprising CAR2 p2a TK SV40 IL12 (p40 t2a p35) (IL-12 Armored CAR 3), or, alternatively, they were not transduced (NC PBMC). Results are shown 3 days (D3), 6 days (D6), or 10 days (D10) post-transduction. Overall, IL-12 “armored” CAR vectors exhibited robust transduction and activity.

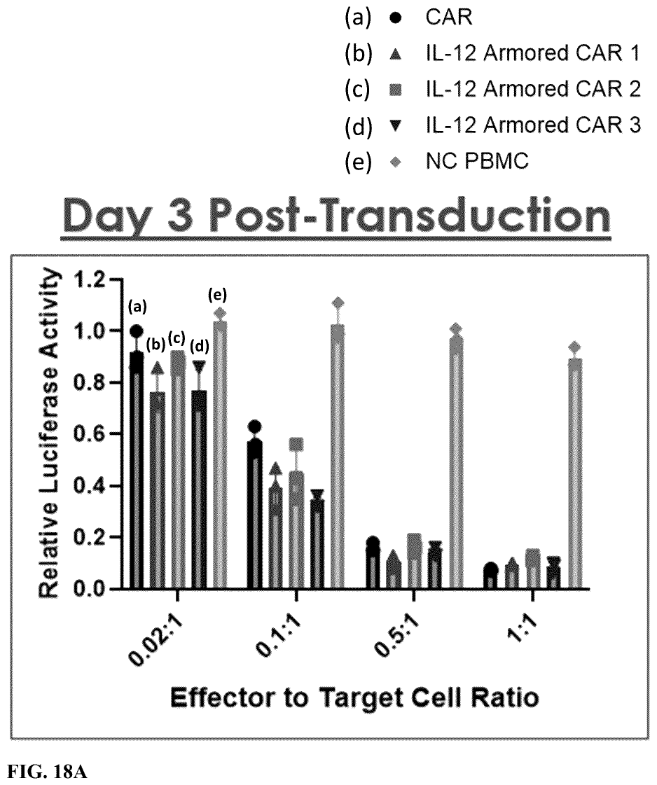

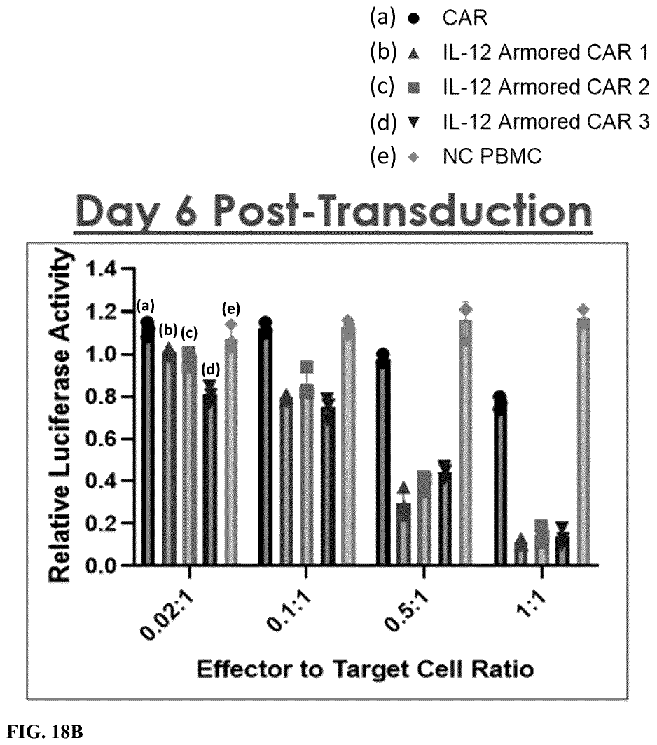

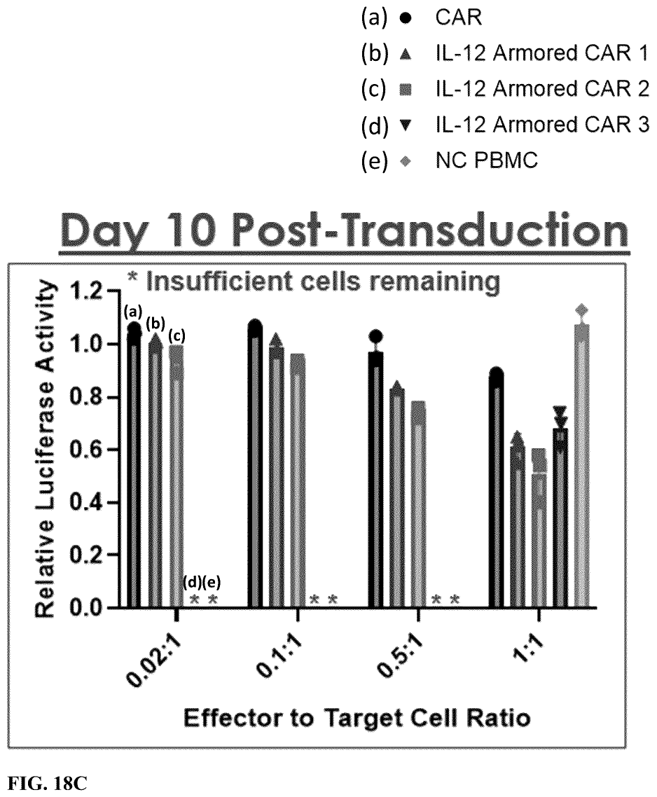

FIGS. 18A-18C show the results of an example reporter gene assay. The assay can demonstrate the magnitude of cytotoxicity of the effector cells, PBMCs, expressing each CAR construct against the target cell line, wherein the level of relative luciferase activity is inversely proportional to cytotoxicity. The effector cells, PBMCs, were transduced with a vector comprising CAR2 p2a TK (CAR) (SEQ ID NO: 38), a vector comprising CAR2 p2a TK IL12 (p40 t2a p35) (IL-12 Armored CAR 1) (SEQ ID NO: 42), a vector comprising CAR2 p2a TK IL12 (p40 tg4sx3 p35) (IL-12 Armored CAR 2) (SEQ ID NO: 43), or a vector comprising CAR2 p2a TK SV40 IL12 (p40 t2a p35) (IL-12 Armored CAR 3) (SEQ ID NO: 40), or, alternatively, they were not transduced (NC PBMC). Effector to target cell ratios of 0.02:1, 0.1:1, 0.5:1, and 1:1 were tested. FIG. 18A shows relative luciferase activity 3 days post-transduction. FIG. 18B shows relative luciferase activity 6 days post-transduction. FIG. 18C shows relative luciferase activity 10 days post-transduction. Overall, IL-12 “armored” CAR vectors exhibited robust transduction and activity, particularly at the more protracted time points.

### Example 4: Murine Surrogate In Vivo CAR Vector Evaluation in C57BL/6 Mice

The transduction efficiency and activity of CAR-expressing vectors provided herein (see, e.g., Example 2) was further evaluated in vivo, particularly in the context of “armored” vectors further expressing IL12. CAR encoding retrovectors with gene cassettes comprised of murine IL-12 genes were created by transient transfection and purified by anion exchange chromatography for administration according to the dose level and schedule outlined in FIG. 19A and Table 5. Briefly, C57BL6/J mice (4 mice per treatment group) were administered an initial intravenous (IV) dose of one of the constructs on day 1. A second IV dose was administered on day 3, and a third IV dose was administered on day 5. Blood samples were taken for analysis by flow cytometry at three days after the final vector dose (day 8) by submandibular bleed and at end of life six days after the final vector dose (day 11) by cardiac stick under isofluorane, per the labeling panel of Table 6 and gating strategy of FIG. 19B. On day 11, end of life, the mice were euthanized, and a terminal blood sample, along with bone marrow, spleen tissue, and liver tissue, were collected.

TABLE 5
Dose Levels and Schedule for CAR
Vector Evaluation in C57BL/6 Mice

| | SEQ | | Dose | Dose |
|---|---|---|---|---|
| | ID | | Concentration | Volume |
| Group | NOs: | Construct | (TU/mL) | (μL) |
| 1 (n = 4) | NA | Vehicle | N/A | 100 μL |
| 2 (n = 4) | 39 | CAR2-P2A-vTK | 1.10E+07 | 100 μL |
| | | (Construct 2 from CAR | | |
| | | STD25-01) | | |
| 3 (n = 4) | 39 | CAR2-P2A-vTK (4X) | 4.30E+07 | 100 μL |
| | | (Construct 2 from CAR | | |
| | | STD25-01) | | |
| 4 (n = 4) | 40 | CAR2-P2A-vTK-SV40- | 4.80E+06 | 100 μL |
| | | mIL 12 | | |
| 5 (n = 4) | 40 | CAR2-P2A-vTK-SV40- | 9.50E+06 | 100 μL |
| | | mIL 12 (2X) | | |
| 6 (n = 4) | 41 | mGEN-1013 (HSV-eTK- | (0.25X) | 100 μL |
| | | SV40-mIL12) | | |
| 7 (n = 4) | 41 | mGEN-1013 (HSV-eTK- | 7.10E+06 (1X) | 100 μL |
| | | SV40-mIL12) | | |

TABLE 6
Flow Cytometry Panel

| | | | | | | | Target | |
|---|---|---|---|---|---|---|---|---|
| Marker | Clone | Fluorophore | Channel | Vendor | Catalog | Dilution | Species | Host |
| PD-1 | 29F.1A12 | BV421 | VL1 | BioLegend | 135221 | 1:100 | Mouse | Rat |
| Live/Dead | N/A | Fixable Aqua | VL3 | Thermo | L34966 | 1:1000 | N/A | N/A |
| CD19 | 1D3 | BV605 | VL4 | Thermo | 406- | 1:100 | Mouse | Rat |
| | | | | | 0193-82 | | | |
| CD45 | 30-F11 | BV711 | BL1 | Thermo | 407- | 1:100 | Mouse | Rat |
| | | | | | 0451-82 | | | |
| CD69 | H1.2F3 | FITC | BL2 | Thermo | 11-0691- | 1:100 | Mouse | Armenian |
| | | | | | 82 | | | hamster |
| CD4 | RM4-5 | Percp-Cy5.5 | BL3 | BioLegend | 100540 | 1:100 | Mouse | Rat |
| hCD19 | N/A | PE | YL1 | Acro | CD9- | 1:50 | Human | N/A |
| scFv | | | | Biosystems | HP2H3 | | | |
| CD8 | 53-6.7 | PE-CF594 | YL2 | BD Horizon | 562283 | 1:100 | Mouse | Rat |
| IFN-γ | XMG1.2 | PE-Cy7 | YL4 | BioLegend | 505826 | 1:100 | Mouse | Rat |
| veTK | N/A | Alexa Fluor | RL1 | Genscript | Custom | 1:200 | N/A | N/A |
| | | 647 | | | Order | | | |
| CD3 | 17A2 | Alexa700 | RL2 | BioLegend | 100216 | 1:100 | Mouse | Rat |
| CD49b | DX5 | APC-e- | RL3 | Thermo | 47-5971- | 1:100 | Mouse | Rat |
| | | Fluor780 | | | 82 | | | |

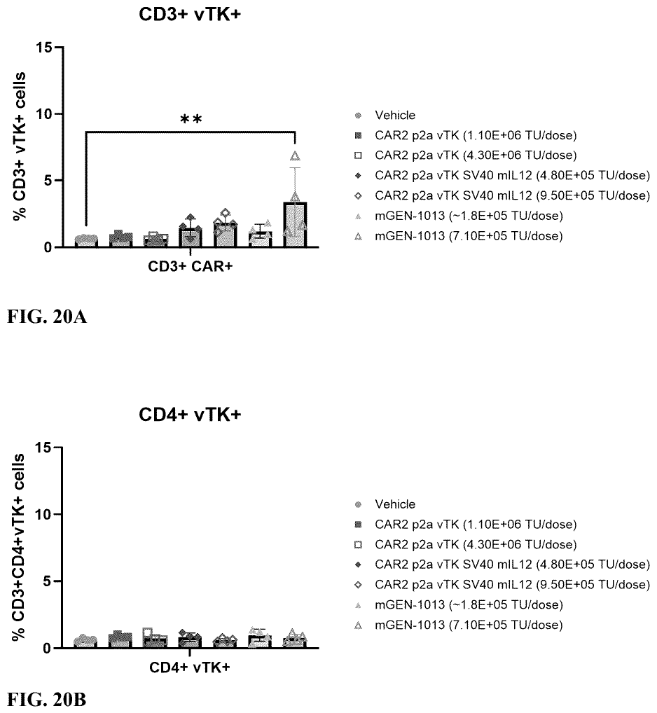

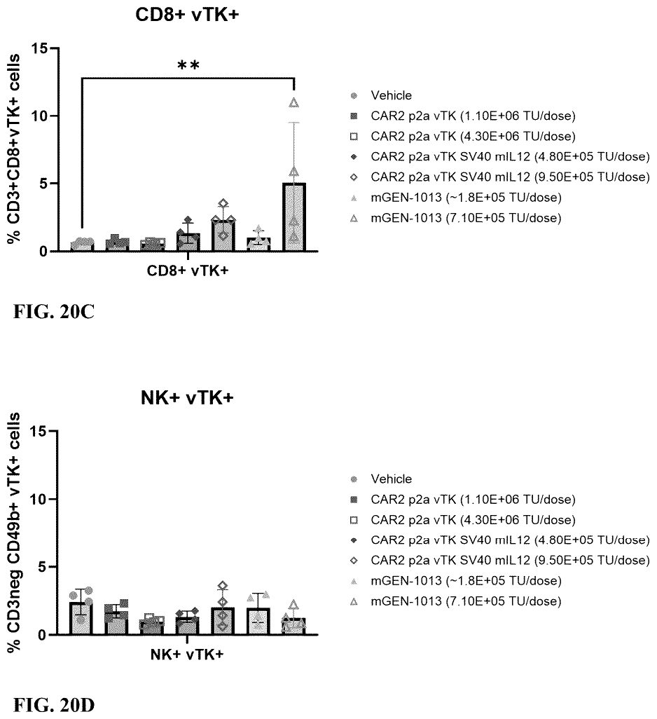

FIGS. 20A-20D show the expression of each construct in CD3+ (FIG. 20A), CD4+ (FIG. 20B), CD8+ (FIG. 20C), or NK+ cells (FIG. 20D) from C57BL/6 mouse submandibular blood samples. The mice were intravenously dosed with 100 μL of each construct, or a vehicle control, on days 1, 3, and 5. A submandibular blood sample was collected on day 11. The higher dose of mGEN-1013 (SEQ ID NO: 41) (i.e., group 7) exhibited the most robust expression in murine blood among the constructs tested.

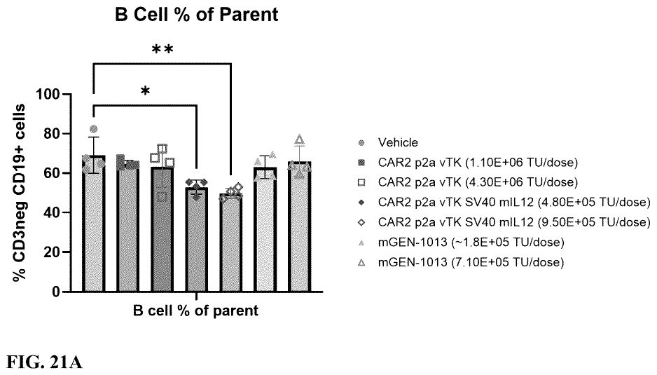

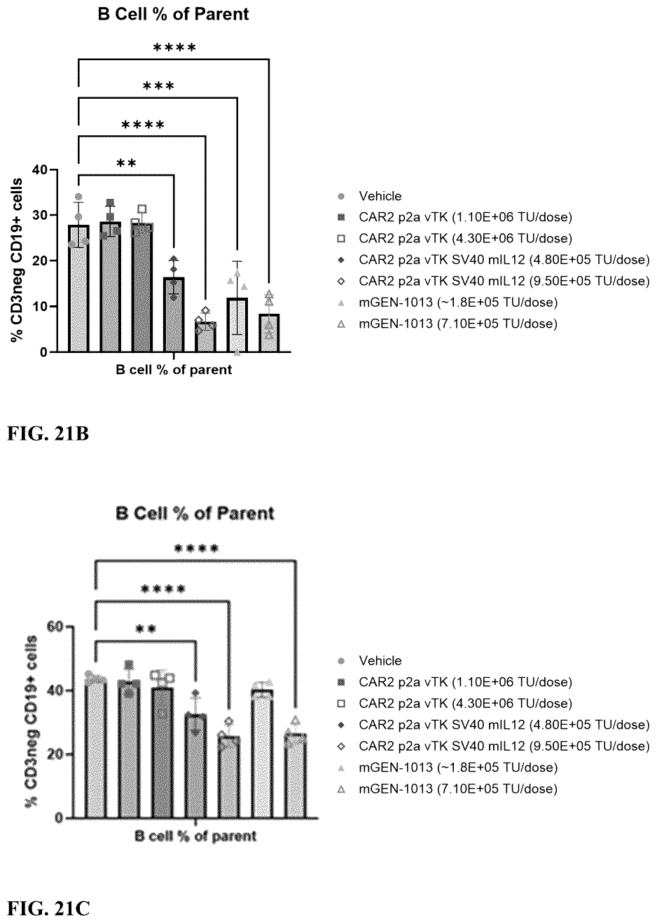

Reduction in B-cell levels following transduction with “armored” CAR-expressing vectors (defined as live, CD45⁺CD3^{neg}CD19+ cells by flow cytometry) was observed in the two groups administered vector encoding for both CAR and IL-12, but not either component in isolation, as shown in FIG. 21A. (*=p<0.05;**=p<0.01 by ordinary 1-way ANOVA). The mice were intravenously dosed with 100 μL of each construct, or a vehicle control, on days 1, 3, and 5. A terminal blood sample was collected on day 11.

IL-12 dependent effects were also observed in B-cell reductions observed in bone marrow and liver samples. FIGS. 21B-21C show the percentage of B cells amongst immune cells from C57BL/6 mouse bone marrow and liver samples, respectively. The mice were intravenously dosed with 100 μL of each construct, or a vehicle control, on days 1, 3, and 5. Bone marrow was collected on day 11. FIG. 21B shows the percentage of B cells (i.e., CD3− CD19+ cells) of parent immune cells from the bone marrow. FIG. 21C shows the percentage of B cells (i.e., CD3− CD19+ cells) of parent immune cells from the liver. The murine bone marrow and liver samples exhibited B cell depletion following transduction with “armored” CAR-expressing vectors.

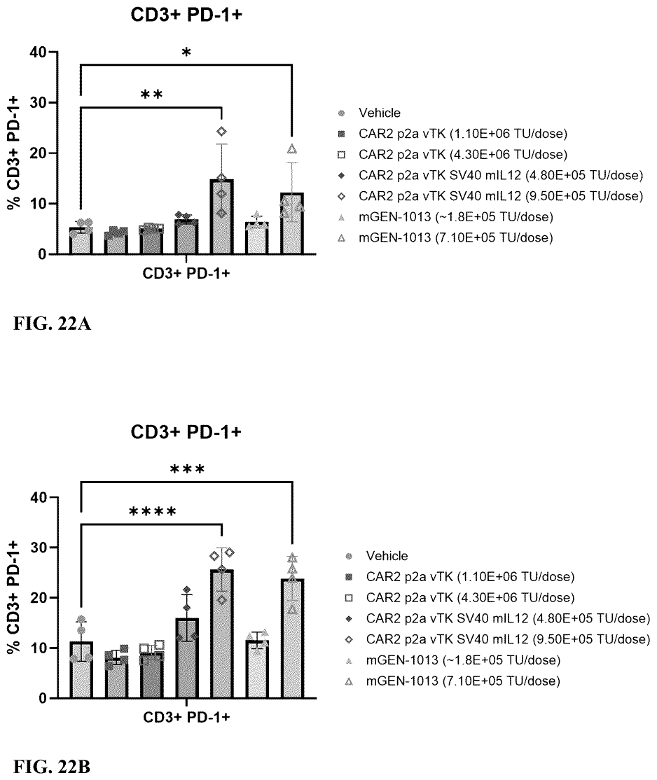

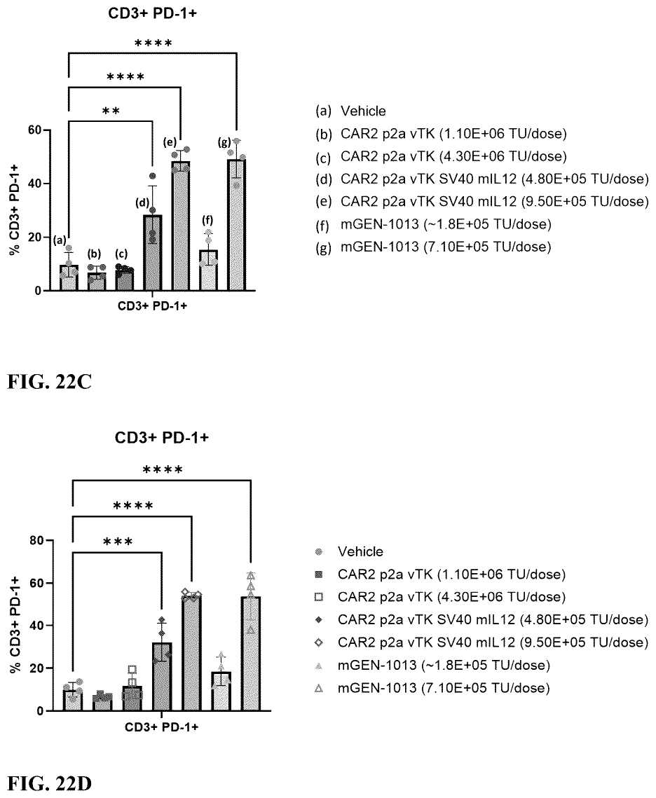

Il-12 dependent effects were further observed in PD-1 increases in T cells (defined as live, CD45+CD3+ cells by flow cytometry) in blood, spleen, bone marrow and liver samples. FIGS. 22A-22D show the magnitude of PD-1 expression in T cells (i.e., CD3+ cells) from C57BL/6 mouse blood (FIG. 22A), spleen (FIG. 22B), bone marrow (FIG. 22C), and liver (FIG. 22D) samples. The mice were intravenously dosed with 100 μL of each construct, or a vehicle control, on days 1, 3, and 5. Blood, spleens, bone marrow, and livers were collected on day 11. PD-1 expression was increased in the T cells of mice administered higher doses of vectors “armored” with IL12 (i.e., groups 5 or 7).

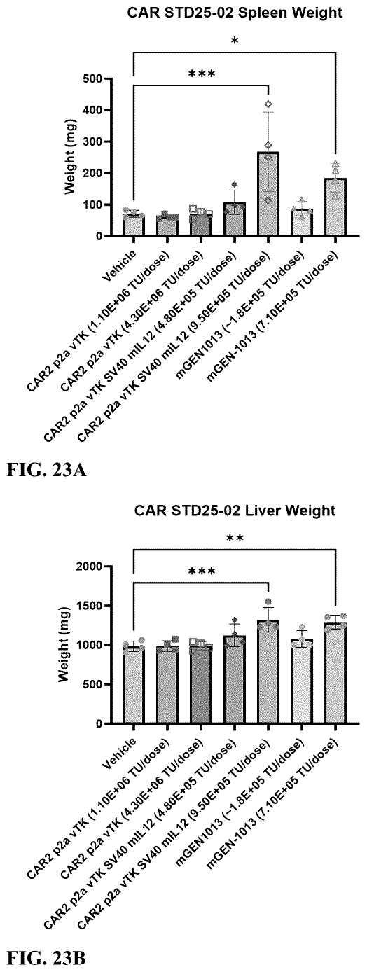

Finally, IL-12 dependent effects were observed in increases in spleen and liver masses on necropsy. FIGS. 23A-23B show spleen (FIG. 23A) or liver (FIG. 23B) weight (mg) from C57BL/6 mice. The mice were intravenously dosed with 100 μL of each construct, or a vehicle control, on days 1, 3, and 5. Spleens and livers were collected on day 11. Mice administered with higher doses of vectors “armored” with IL12 (i.e., groups 5 or 7) exhibited increased spleen and liver weights. A significant (p<0.05) reduction in B-cell levels was obtained with the SV40-driven IL-12 at a dose level where spleen and liver enlargements were not significant (FIGS. 23A-23B).

### Example 5: Viral Vectors with a Modified Sindbis Virus Envelope Specifically Transduce CD8+ Cells

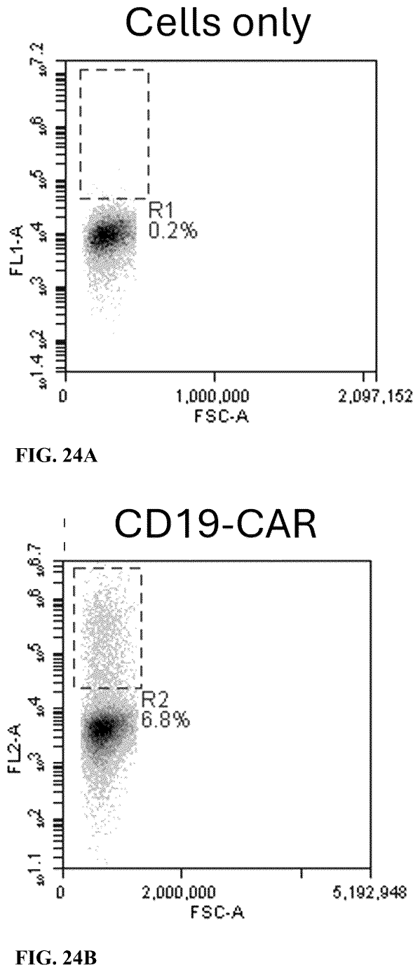

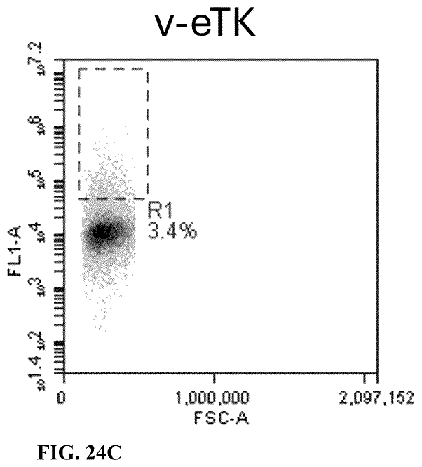

Murine leukemia virus (MLV)-based vectors with a modified Sindbis virus envelope (SEQ ID NO: 1) were generated. The modified Sindbis virus envelope had mutations that ablate the binding of the wild-type Sindbis virus envelope to its natural receptors, laminin receptors. A CD8 targeting motif (scFv) was inserted into the E2 domain of the modified Sindbis envelope to generate a CD8-targeted vector. A schematic structure of example vectors similar to those used is shown in FIG. 13. Using the vector carrying CAR-TK as the payload and a CD8-targeting envelope (SEQ ID NO: 1), a human cell line SUP-T1 (CD8+) was transduced. CAR and TK expression was examined 3 days post-transduction. FIGS. 24A-24C shows the transduction of SUP-T1 cells with CD8-targeted CAR-TK vectors. The different promoters driving expression of CAR (LTR promoter) and TK (SV40) may have influenced the expression level of each protein.

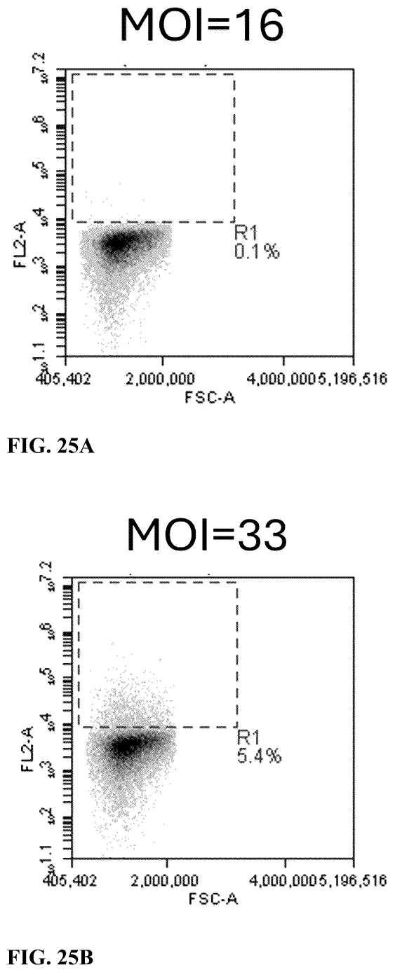

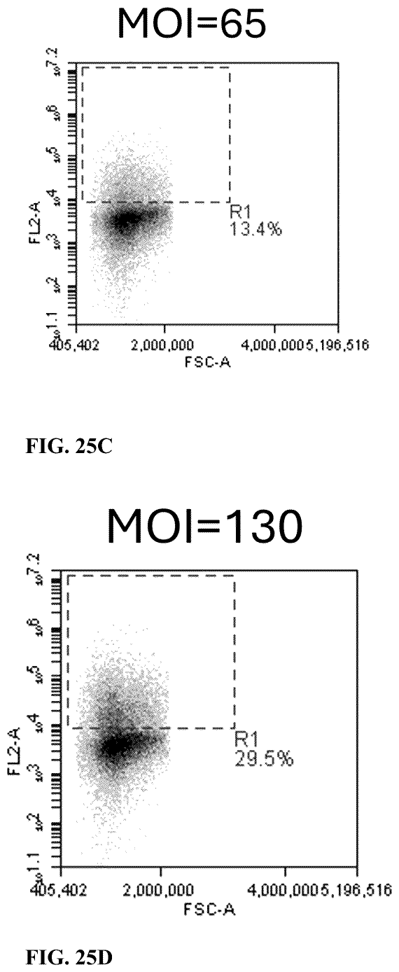

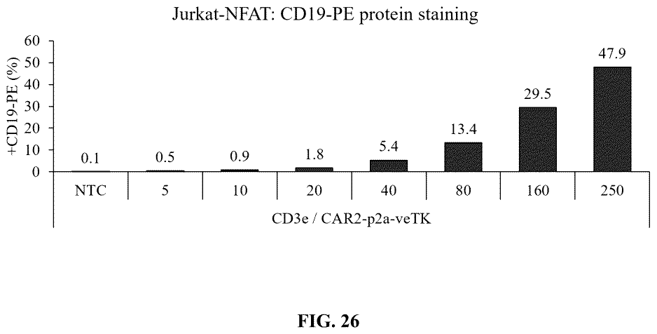

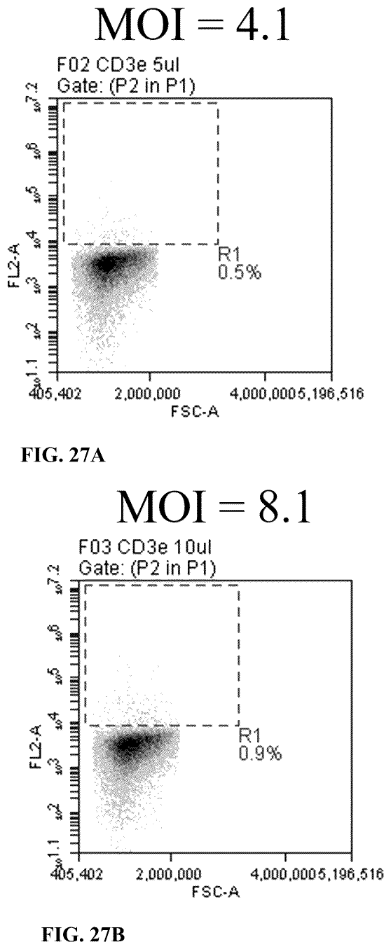

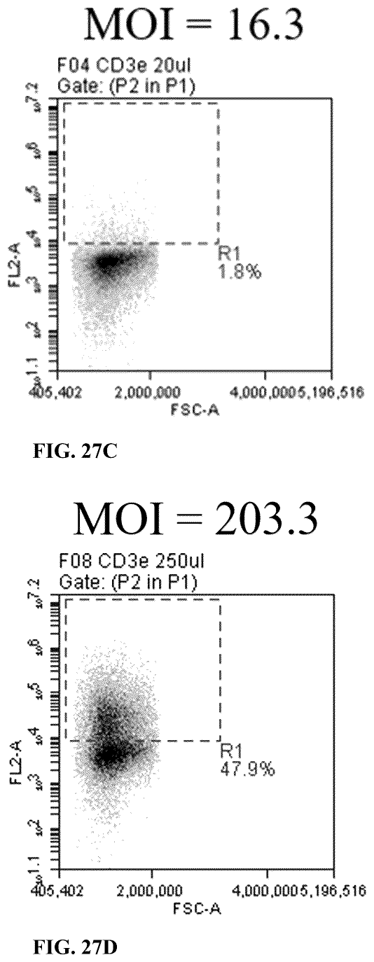

Similarly, a CD3 targeting motif (scFv) was inserted into the E2 domain of the modified Sindbis envelope to generate a CD3-targeted vector. Using the purified vectors carrying CAR-TK as the payload and a CD3-targeting envelope, a human cell line Jurkat E6 (CD3+) was transduced with different multiplicities of infection (MOIs). CAR expression was examined 3 days post-transduction. FIGS. 25A-25D show the dose-dependent transduction of Jurkat cells by flow cytometry. FIG. 26 shows the magnitude of CD19-CAR expression (i.e., +CD19-PE (%)) in Jurkat cells following transduction with ascending MOIs of SB-pseudotyped MLV CAR vector with scFv-CD3-targeted envelope (SEQ ID NO: 45). Transduction was performed via Retronectin and spinoculation. Specifically, vectors and 1×10⁵ Jurkat cells per well were placed in pre-incubation Retronectin coated plates for 2 hours at 37° C. Viral vectors and the cells to be transduced can both bind to the Retronectin-coated surface. Spinoculation was performed at 1800 rpm for 45 minutes. This can increase their proximity and interaction time, which can lead to more efficient gene transfer. MOIs of 4.1, 8.1, 16.3, 32.5, 65, 130.1, and 203.3 were used for transduction. FIGS. 27A-27D show the results from a FACS experiment demonstrating example CD19-CAR expression in Jurkat cells, a CD3-positive cell line, which were transduced with ascending MOIs of SB-pseudotyped MLV CAR vector with scFv-CD3-targeted envelope (SEQ ID NO: 39).

Odronextamab-based scFv-CD3 with linkers (in Sindbis env):

(linker5)
(SEQ ID NO: 36)
GGGGSGGGGSEVQLVESGGGLVQPGRSLRLSCAASGFTFDDYTMHWVRQ
APGKGLEWVSGISWNSGSIGYADSVKGRFTISRDNAKKSLYLQMNSLRA
EDTALYYCAKDNSGYGHYYYGMDVWGQGTTVTVASASTKGGGGGSGGGG
SGGGGSEIVMTQSPATLSVSPGERATLSCRASQSVSSNLAWYQQKPGQA
PRLLIYGASTRATGIPARFSGSGSGTEFTLTISSLQSEDFAVYYCQHYI
NWPLTFGGGTKVEIKSGGGGSGGGG
(linker6)
(SEQ ID NO: 37)
AAGHVGEVQLVESGGGLVQPGRSLRLSCAASGFTFDDYTMHWVRQAPGK
GLEWVSGISWNSGSIGYADSVKGRFTISRDNAKKSLYLQMNSLRAEDTA
LYYCAKDNSGYGHYYYGMDVWGQGTTVTVASASTKGGGGGSGGGGSGGG
GSEIVMTQSPATLSVSPGERATLSCRASQSVSSNLAWYQQKPGQAPRLL
IYGASTRATGIPARFSGSGSGTEFTLTISSLQSEDFAVYYCQHYINWPL
TFGGGTKVEIKGVHGAA

While preferred embodiments of the present disclosure have been shown and described herein, it will be obvious to those skilled in the art that such embodiments are provided by way of example only. Numerous variations, changes, and substitutions will now occur to those skilled in the art without departing from the disclosure. It should be understood that various alternatives to the embodiments of the present disclosure may be employed in practicing the present disclosure. It is intended that the following claims define the scope of the present disclosure and that methods and structures within the scope of these claims and their equivalents be covered thereby.

1.-48. (canceled)

49. A method of expressing a chimeric antigen receptor (CAR) in a cell of a subject comprising contacting the cell of the subject with an engineered viral vector comprising a first payload vector encoding a chimeric antigen receptor (CAR), under conditions suitable to express the CAR in the cell of the subject.

50. A method of treating or preventing a disease or condition in a subject comprising administering to the subject an engineered viral vector comprising a first payload vector encoding a chimeric antigen receptor (CAR), wherein expression of the CAR affects the disease or condition in the subject.

51. The method of claim 50, wherein the disease or condition comprises a cancer, an infectious disease, an inflammatory disease, or an autoimmune disease.

52. The method of claim 49, wherein the engineered viral vector comprises an engineered retroviral vector comprising a nucleic acid sequence encoding a deleted or defective integrase.

53. The method of claim 49, wherein the engineered viral vector further comprises a second payload vector encoding a suicide gene or a cytokine.

54. The method of claim 53, wherein the suicide gene comprises HSV1-TK.

55. The method of claim 53, wherein the cytokine comprises interleukin-2 (IL-2), interleukin-7 (IL-7), interleukin-12 (IL-12), interleukin-15 (IL-15), interleukin-18 (IL-18), interleukin-21 (IL-21), interferon-gamma (IFN-7), tumor necrosis factor-alpha (TNF-α), interleukin-6 (IL-6), or a combination thereof.

56. The method of claim 49, wherein the engineered viral vector comprises a modified envelope protein, wherein:
(a) the modified envelope protein comprises a targeting moiety; and
(b) the modified envelope protein increases transduction specificity of the engineered viral vector to a target cell compared to an otherwise identical viral vector without the modified envelope protein.

57. The method of claim 56, wherein the modified envelope protein comprises a recombinant viral envelope protein derived from a Sindbis virus comprising an E3 domain, an E2 domain, a 6K domain, an E1 domain, or a combination thereof.

58. The method of claim 56, wherein the targeting moiety comprises a single-chain variable fragment (scFv), a diabody, a single variable domain on a heavy chain (V_{HH}), a ligand that binds to a receptor, or a combination thereof.

59. The method of claim 58, wherein the scFv targets CD3, CD8, or a combination thereof.

60. The method of claim 58, wherein the scFv is encoded by a sequence with at least 60%, at least 70%, at least 75%, at least 80%, at least 85%, at least 90%, at least 95%, or at least 99% sequence identity to SEQ ID NO: 2.

61. The method of claim 50, wherein the engineered viral vector comprises an engineered retroviral vector comprising a nucleic acid sequence encoding a deleted or defective integrase.

62. The method of claim 50, wherein the engineered viral vector further comprises a second payload vector encoding a suicide gene or a cytokine.

63. The method of claim 62, wherein the suicide gene comprises HSV1-TK.

64. The method of claim 62, wherein the cytokine comprises interleukin-2 (IL-2), interleukin-7 (IL-7), interleukin-12 (IL-12), interleukin-15 (IL-15), interleukin-18 (IL-18), interleukin-21 (IL-21), interferon-gamma (IFN-7), tumor necrosis factor-alpha (TNF-α), interleukin-6 (IL-6), or a combination thereof.

65. The method of claim 50, wherein the engineered viral vector comprises a modified envelope protein, wherein:
(a) the modified envelope protein comprises a targeting moiety; and
(b) the modified envelope protein increases transduction specificity of the engineered viral vector to a target cell compared to an otherwise identical viral vector without the modified envelope protein.

66. The method of claim 65, wherein the modified envelope protein comprises a recombinant viral envelope protein derived from a Sindbis virus comprising an E3 domain, an E2 domain, a 6K domain, an E1 domain, or a combination thereof.

67. The method of claim 65, wherein the targeting moiety comprises a single-chain variable fragment (scFv), a diabody, a single variable domain on a heavy chain (V_{HH}), a ligand that binds to a receptor, or a combination thereof.

68. The method of claim 67, wherein the scFv targets CD3, CD8, or a combination thereof.
